SEQUENCE LISTING (17 sequences)

SEQ ID NO: 1 - protein, 484 aa, Homo sapiens
MGSAFERVVRRVVQELDHGGEFIPVTSLQSSTGFQPYCLVVRKPSSSWFWKPRYKCVNLSIKDILEPDAAEPDVQRGRSFHFYDAMDGQIQGSVELAAPGQAKIAGGAAVSDSSSTSMNVYSLSVDPNTWQTLLHERHLRQPEHKVLQQLRSRGDNVYVVTEVLQTQKEVEVTRTHKREGSGRFSLPGATCLQGEGQGHLSQKKTVTIPSGSTLAFRVAQLVIDSDLDVLLFPDKKQRTFQPPATGHKRSTSEGAWPQLPSGLSMMRCLHNFLTDGVPAEGAFTEDFQGLRAEVETISKELELLDRELCQLLLEGLEGVLRDQLALRALEEALEQGQSLGPVEPLDGPAGAVLECLVLSSGMLVPELAIPVVYLLGALTMLSETQHKLLAEALESQTLLGPLELVGSLLEQSAPWQERSTMSLPPGLLGNSWGEGAPAWVLLDECGLELGEDTPHVCWEPQAQGRMCALYASLALLSGLSQEPH

SEQ ID NO: 2 - protein, 275 aa, Homo sapiens
MGSAFERVVRRVVQELDHGGEFIPVTSLQSSTGFQPYCLVVRKPSSSWFWKPRYKCVNLSIKDILEPDAAEPDVQRGRSFHFYDAMDGQIQGSVELAAPGQAKIAGGAAVSDSSSTSMNVYSLSVDPNTWQTLLHERHLRQPEHKVLQQLRSRGDNVYVVTEVLQTQKEVEVTRTHKREGSGRFSLPGATCLQGEGQGHLSQKKTVTIPSGSTLAFRVAQLVIDSDLDVLLFPDKKQRTFQPPATGHKRSTSEGAWPQLPSGLSMMRCLHNFLTD

SEQ ID NO: 3 - protein, 209 aa, Homo sapiens
GVPAEGAFTEDFQGLRAEVETISKELELLDRELCQLLLEGLEGVLRDQLALRALEEALEQGQSLGPVEPLDGPAGAVLECLVLSSGMLVPELAIPVVYLLGALTMLSETQHKLLAEALESQTLLGPLELVGSLLEQSAPWQERSTMSLPPGLLGNSWGEGAPAWVLLDECGLELGEDTPHVCWEPQAQGRMCALYASLALLSGLSQEPH

SEQ ID NO: 4 - protein, 115 aa, Homo sapiens
PNTWQTLLHERHLRQPEHKVLQQLRSRGDNVYVVTEVLQTQKEVEVTRTHKREGSGRFSLPGATCLQGEGQGHLSQKKTVTIPSGSTLAFRVAQLVIDSDLDVLLFPDKKQRTFQ

SEQ ID NO: 5 - protein, 95 aa, Homo sapiens
RHLRQPEHKVLQQLRSRGDNVYVVTEVLQTQKEVEVTRTHKREGSGRFSLPGATCLQGEGQGHLSQKKTVTIPSGSTLAFRVAQLVIDSDLDVLL

SEQ ID NO: 6 - protein, 75 aa, Homo sapiens
LQQLRSRGDNVYVVTEVLQTQKEVEVTRTHKREGSGRFSLPGATCLQGEGQGHLSQKKTVTIPSGSTLAFRVAQL

SEQ ID NO: 7 - protein, 55 aa, Homo sapiens
VYVVTEVLQTQKEVEVTRTHKREGSGRFSLPGATCLQGEGQGHLSQKKTVTIPSG

SEQ ID NO: 8 - protein, 35 aa, Homo sapiens
QKEVEVTRTHKREGSGRFSLPGATCLQGEGQGHLS

SEQ ID NO: 9 - protein, 15 aa, Homo sapiens
KREGSGRFSLPGATC

SEQ ID NO: 10 - protein, 13 aa, Homo sapiens
EHKVLQQLRSRGD

SEQ ID NO: 11 - protein, 14 aa, Homo sapiens
AFERVVRRVVQELD

SEQ ID NO: 12 - protein, 6 aa, Homo sapiens
YCLVVR

SEQ ID NO: 13 - protein, 7 aa, Homo sapiens
YKCVNLS

SEQ ID NO: 14 - protein, 7 aa, Homo sapiens
MDGQIQG

SEQ ID NO: 15 - protein, 10 aa, Homo sapiens
PNTWQTLLHE

SEQ ID NO: 16 - protein, 4 aa, Homo sapiens
DLDV

SEQ ID NO: 17 - protein, 15 aa, Mus musculus
SQEGSGQFTLPGALC

(19) United States
(12) Patent Application Publication
(10) Pub. No.: US 2022/0275069 A1
(43) Pub. Date: Sep. 1, 2022

(54) BINDING AGENTS AND USES THEREOF

(71) Applicant: Janssen Pharmaceutica NV, Beerse (BE)

(72) Inventors: Mohamed LAMKANFI, Ghent (BE); Filip Frans J. VAN HAUWERMEIREN, Beerse (BE)

(21) Appl. No.: 17/626,200

(22) PCT Filed: Jul. 10, 2020
(86) PCT No.: PCT/EP2020/069667

§ 371 Date: Jan. 11, 2022

(30) Foreign Application Priority Data
Jul. 12, 2019 (EP) 19186059.2

(51) Int. Cl. C07K 16/18; A61P 35/00

(52) U.S. Cl. CPC C07K 16/18; A61P 35/00; A61K 2039/505

(57) ABSTRACT

The present invention relates to inhibitors that bind to gasdermin D and inhibit gasdermin D-mediated cell death and inflammation. The inhibitors can be proteinaceous, for example antigen binding proteins or non-proteinaceous, such as aptamers. It is disclosed an antibody that binds to the peptide of SEQ ID NO: 9: KREGSGRFSLPGATC. In one embodiment the inhibitor binds to gasdermin D and inhibits its association with lipids, such as wherein the inhibitor binds to gasdermin D and neutralizes the association of gasdermin D with lipids, optionally wherein the inhibitor binds to gasdermin D and inhibits its association with phosphatidyl inositol 4-phosphate and/or phosphatidyl inositol 4,5-bisphosphate, such as wherein the inhibitor binds to gasdermin D and neutralizes the association of gasdermin D with phosphatidylinositol 4-phosphate and/or phosphatidyl inositol 4,5-bisphosphate.

### TECHNICAL FIELD

The disclosure provided herein relates to inhibitors that bind to gasdermin D and inhibit gasdermin D-mediated cell death and inflammation.

### BACKGROUND

Pyroptosis is a highly inflammatory type of programmed cell death that is triggered by a number of different threats, including microbial pathogens or host-derived perturbations of the cytosol. Pyroptosis is morphologically and mechanistically distinct from other forms of cell death, and is dependent on activation and recruitment of the protein gasdermin D to form multimeric pores in the cell membrane. The formation of these multimeric pores disrupts the membrane potential of the cell, dissipating cellular ionic gradients, and producing a net increase in osmotic pressure, water influx, cell swelling and eventually osmotic lysis. The gasdermin D pores are able to release inflammatory cytokines such as interleukin (IL)-1α, IL-1β and IL-18, and the cell death that is triggered by osmotic lysis triggers further release of inflammatory cytokines and alarmins such as HMGB1 and ATP. Characteristics of pyroptosis therefore include rapid plasma-membrane rupture and release of inflammatory cytokines, which further recruit immune effector cells that activates a larger inflammatory response in the host (Pandeya et al., (2019) Med. Chem. Commun. 10(5): 660-667).

Pyroptosis is therefore a primary cellular response that protects against infection and induces pathological inflammation. However, the dysregulation of the inflammatory response is a key driver in many debilitating diseases linked to acute and chronic inflammation such as sepsis, atherosclerosis, inflammatory bowel disease, non-alcoholic steatohepatitis, lung cancer, familial Mediterranean fever and autoinflammatory diseases such as cryopyrin-associated periodic syndromes. Inhibitors that specifically target pyroptotic cell death may therefore be therapeutically useful in the clinic for the treatment of these diseases.

The NLRP3 inflammasome and caspase 1 are key inflammatory response initiators of pyroptosis. Other inflammasomes, such as AIM2, NLRC4 and NLRP1 can also induce activation of caspase-1, gasdermin D and pyroptosis. Once activated by the inflammasome complex, caspase 1 initiates a proinflammatory response through the cleavage and activation of cytokines, such as IL-1β and IL-18, as well as the cleavage of the protein gasdermin D. Caspase 1 independent pyroptosis can also proceed via the caspases 4, 5 and 8 which can also cleave gasdermin D (Bergsbaken et al., (2009) Nat Rev Microbiol 7(2): 99-109; Frank and Vince (2019) Cell Death and Differentiation 26: 99-114).

Gasdermin D, also known as GSDMD, DF5L, DFNA5L, GSDMDC1 and FKSG10, is a ˜53 kDa protein that is composed of an N-terminal and C-terminal domain. The N-terminal domain (GSDMD^{Nterm}) is 31 kDa and separated by a linker to the C-terminal domain (GSDMD^{Cterm}) which is 22 kDa. The full length protein is in an autoinhibited state, with the C-terminal domain folding back and intramolecularly binding to the N-terminal domain. Cleavage of the full length gasdermin D in the region of the interdomain linker by the proinflammatory proteases, results in the separation of the N- and C-terminal domains of gasdermin D which lifts the autoinhibition. GSDMD^{Nterm}, by itself, is able to induce pyroptosis.

There are no current therapeutics on the market that directly target gasdermin D for the treatment of sepsis or other inflammatory diseases. Research targeting gasdermin D has focused only on small molecule inhibitors that are able to react with a free thiol at position Cys191 of gasdermin D (Rathekey et al., (2018) Sci. Immunology. 3, eaat2738). Accordingly there is a need for new therapeutic modalities.

### SUMMARY OF THE INVENTION

The invention provides a gasdermin D inhibitor that binds to gasdermin D and inhibits gasdermin D, such as wherein the inhibitor binds to gasdermin D and neutralizes gasdermin D.

The invention also provides a gasdermin D inhibitor that binds to gasdermin D and neutralizes gasdermin D.

The invention also provides a method for inhibiting and/or neutralizing an activity and/or function of gasdermin D, comprising contacting the gasdermin D inhibitor of the invention with gasdermin D.

The invention also provides use of the gasdermin D inhibitor of the invention in inhibiting and/or neutralizing an activity and/or function of gasdermin D.

The invention also provides an inhibitor of gasdermin D for use in inhibiting and/or neutralizing gasdermin D, such as neutralizing gasdermin D, optionally wherein the inhibition and/or neutralization of gasdermin D is the inhibition and/or neutralization of:
i) an activity of gasdermin D, such as wherein the inhibitor neutralizes an activity of gasdermin D; and/or
ii) a function of gasdermin D, such as wherein the inhibitor neutralizes a function of gasdermin D.

The invention also provides an inhibitor of gasdermin D for use in therapy, for instance for use in the treatment of an indication selected from: sepsis, septic shock, non-alcoholic steatohepatitis, lung cancer, Familial Mediterranean Fever, autoinflammatory diseases, Cryoprin associated periodic syndromes, non-alcoholic fatty liver disease, Alzheimer's disease, Parkinson's disease, age related macular degeneration, atherosclerosis, asthma and allergy airway inflammation, gout, Crohn's, ulcerative colitis, inflammatory bowel disease, hypertension, nephropathy, myocardial infarction, multiple sclerosis, experimental autoimmune encephalitis, hyperinflammation following influenza infection, graft versus host disease, stroke, silicosis, asbestosis, mesothelioma, type 1 diabetes, type 2 diabetes, obesity-induced inflammation, insulin resistance, rheumatoid arthritis, myelodysplastic syndrome, contact hypersensitivity, joint inflammation triggered by chikungunya virus and traumatic brain injury.

### DETAILED DESCRIPTION

### Inhibitors of Gasdermin D

The invention provides a gasdermin D inhibitor that binds to gasdermin D and inhibits gasdermin D, such as wherein the inhibitor neutralizes gasdermin D.

The invention provides a gasdermin D inhibitor that binds to gasdermin D and neutralizes gasdermin D.

Accordingly, the invention provides a gasdermin D inhibitor that binds to gasdermin D and inhibits gasdermin D, wherein the inhibition of gasdermin D is the inhibition of an activity of gasdermin D, such as wherein the inhibitor neutralizes an activity of gasdermin D and/or the inhibition of gasdermin D is the inhibition of a function of gasdermin D, such as wherein the inhibitor neutralizes a function of gasdermin D.

Accordingly, the invention provides a gasdermin D inhibitor that binds to gasdermin D and neutralizes gasdermin D, wherein the neutralization of gasdermin D is the neutralization of an activity of gasdermin D, and/or the neutralization of gasdermin D is the neutralization of a function of gasdermin D.

Accordingly, the invention provides an extracellular inhibitor that binds to gasdermin D. Accordingly, the invention provides an inhibitor that binds to gasdermin D on the cell surface.

Accordingly, the invention also provides an inhibitor that binds to gasdermin D and does not cross the cell membrane, unless it is bound to gasdermin D.

Accordingly, the invention also provides an inhibitor of gasdermin D that is a large molecule.

Accordingly the invention also provides, an inhibitor that binds to gasdermin D, wherein the inhibitor has a molecular weight of >2 kDa, >3 kDa, >4 kDa, >5 kDa, >6 kDa, >7 kDa, >8 kDa, >9 kDa or >10 kDa.

The proteolytic cleavage of gasdermin D results in the insertion of the N-terminal domain of gasdermin D into the cell membrane. Accordingly, in some embodiments the inhibitor of the invention binds to the N-terminal domain of gasdermin D. In some embodiments the inhibitor of the invention binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the inhibitor of the invention binds to an epitope comprising SEQ ID NO: 9. In some embodiments, the inhibitor of the invention binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the inhibitor of the invention binds to SEQ ID NO: 9.

In some embodiments the inhibitor of the invention binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the inhibitor of the invention binds to an isolated peptide of SEQ ID NO: 9.

The insertion of gasdermin D into the plasma membrane requires the association of the N-terminal domain of gasdermin D with lipids of the cell membrane. Accordingly, in some embodiments the inhibitor of the invention binds to gasdermin D and inhibits its association with lipids, such as wherein the inhibitor binds to gasdermin D and neutralizes the association of gasdermin D with lipids. In some embodiments the inhibitor of the invention binds to gasdermin D and inhibits the association of gasdermin D with phosphatidylinositol 4-phosphate and/or phosphatidylinositol 4,5-bisphosphate, such as wherein the inhibitor binds to gasdermin D and neutralizes the association of gasdermin D with phosphatidylinositol 4-phosphate and/or phosphatidylinositol 4,5-bisphosphate.

The insertion of the N-terminal domain of gasdermin D in the cell membrane results in the oligomerisation of gasdermin D subunits, where it functions as a multimeric pore complex. The inventors have determined that the function of gasdermin D can be disrupted by preventing pore formation. In some embodiments, the inhibitor of the invention binds to gasdermin D and inhibits oligomerisation of gasdermin D, such as wherein the inhibitor binds to gasdermin D and neutralizes oligomerisation of gasdermin D. This mechanism thereby prevents formation of the pore.

The oligomerisation of gasdermin D subunits to form the pore requires interaction between gasdermin D subunits. Disruption of protein-protein interactions between gasdermin D subunits will therefore inhibit pore formation. Accordingly, in some embodiments, the inhibitor of the invention inhibits protein-protein interactions between the gasdermin D subunits, such as wherein the inhibitor neutralizes protein-protein interactions between the gasdermin D subunits. This mechanism thereby prevents oligomerisation.

The formation of the gasdermin D multimeric pore complex leads to the release of cytokines and disruption of cellular ionic gradients. Disruption and inhibition of pores that have already formed on the cell surface can inhibit gasdermin D activity/activities, such as cell death by pyroptosis. Accordingly, in some embodiments, the inhibitor of the invention binds to a gasdermin D multimeric pore. In some embodiments, the inhibitor of the invention binds to a gasdermin D multimeric pore and blocks the pore. In some embodiments, the inhibitor of the invention binds to a gasdermin D multimeric pore and disrupts protein-protein interactions between subunits of the pore. In some embodiments, the inhibitor of the invention binds to a gasdermin D subunit of the multimeric pore. In some embodiments, the inhibitor of the invention binds to a gasdermin D subunit of the multimeric pore, and blocks the pore. In some embodiments, the inhibitor of the invention binds to a gasdermin D subunit of the multimeric pore, and disrupts the protein-protein interactions between the subunits of the pore.

In some embodiments the inhibitor of the invention inhibits release of IL-1β and/or IL-18, such as wherein the inhibitor neutralizes release of IL-1β and/or IL-18.

The inhibition of an activity and/or function of gasdermin D can result in the inhibition of cell death due to pyroptosis. Accordingly, in some embodiments the inhibitor of the invention can inhibit cell death induced by pyroptosis. In some embodiments the inhibitor of the invention inhibits cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein. In some embodiments, the inhibitor of the invention, when administered to human THP-1 cells, inhibits cell death induced by pyroptosis by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl (for example 50,000 cells or 65,000 cells in 100 μl), wherein the THP-1 cells have been pre-treated with phorbol 12-myristate 13-acetate for 16 hours, wherein the THP-1 cells have been induced with 20 μM nigericin, and wherein cell death is measured after 1 hour.

The inhibition of an activity and/or function of gasdermin D can result in the delay of cell death, such as due to pyroptosis. Accordingly, in some embodiments the inhibitor of the invention can delay cell death induced by pyroptosis by >0.1 hours, >0.2 hours, >0.5 hours, >0.75 hours, >1 hour, >2 hours, >3 hours, >4 hours, or >6 hours. In some embodiments the inhibitor of the invention, when administered to human THP-1 cells, delays cell death induced by pyroptosis by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl (for example 50,000 cells or 65,000 cells in 100 μl), wherein the THP-1 cells have been pre-treated with phorbol 12-myristate 13-acetate for 16 hours, and wherein the THP-1 cells have been induced with 20 μM nigericin.

The development of inhibitors for use in therapy require pre-clinical testing prior to authorization. An inhibitor of the invention that cross reacts with an old world monkey gasdermin D or a new world monkey gasdermin D, therefore allows experimental testing in non-human primates, and provides a non-human model to test the toxicological, safety and efficacy profiles of the inhibitor of the invention. This therefore can increase development efficiency. Accordingly in some embodiments the inhibitor of the invention cross reacts with an old world monkey gasdermin D or a new world monkey gasdermin D.

The invention provides a gasdermin D inhibitor that binds to gasdermin D and inhibits gasdermin D, such as wherein the inhibitor binds to gasdermin D and neutralizes gasdermin D, wherein the inhibition and/or neutralization of gasdermin D is the inhibition and/or neutralization of:
i) an activity of gasdermin D, such as wherein the inhibitor neutralizes an activity of gasdermin D; and/or
ii) a function of gasdermin D, such as wherein the inhibitor neutralizes a function of gasdermin D;
   and wherein:
a) the inhibitor is an extracellular inhibitor;
b) the inhibitor binds to the N-terminal domain of gasdermin D;
c) the inhibitor binds to an epitope comprising SEQ ID NO: 9; and
d) the inhibitor binds to SEQ ID NO: 9.

### Antigen Binding Proteins

In some embodiments, the inhibitor of the invention is proteinaceous, for example an antigen binding protein. Antigen binding proteins are any proteinaceous structure that may exhibit binding affinity for a particular antigen. Herein, the specified target antigen is the gasdermin D protein or fragment thereof. “Antigen binding protein” includes but is not limited to antibodies and binding parts thereof, such as immunologically functional fragments. Peptibodies (Fc-fusions) are another example of antigen binding proteins, as are heavy and light chain derived antibody fragments. Such fragments are active as they can bind to the epitope and inhibit gasdermin D. In one aspect, such a fragment will retain at least one complementarity determining region (CDR) present in the full-length light or heavy chain, and in some embodiments will comprise a single heavy chain and/or light chain or portion thereof. Fragments can be easily produced by enzyme or chemical cleavage. They include, but are not limited to, Fab, a diabody, Fab′, F(ab′)2, Fv, domain antibodies and single-chain antibodies. Some antigen binding proteins described herein are antibodies or are derived from antibodies. In some embodiments, the polypeptide structure of the antigen binding proteins is based on antibodies, including, but not limited to, monoclonal antibodies, bispecific antibodies, minibodies, domain antibodies, chimeric antibodies, humanized antibodies, human antibodies, antibody fusions (sometimes referred to as “antibody conjugates”), and fragments thereof, respectively. Additionally, antigen-binding fragments may include non-antibody proteinaceous frameworks that may successfully incorporate polypeptide segments in an orientation that confers affinity for a given antigen of interest, such as protein scaffolds. Non-limiting examples of such protein scaffolds include: a nanobody (VHH) domain, an IgNAR variable domain (vNAR), a Fc fusion protein, a variable lymphocyte receptor (VLR) domain, a Fibronectin type III domain, a centyrin, a Kringle domain protein, a designed ankyrin repeat protein (DARPin), a cystine-knot miniprotein, a Sso7d protein, an affibody, an affimer, an anticalin, an affilin, an affitin, or a fynomer, as discussed in more detail below.

The invention provides an antigen binding protein that binds to gasdermin D and inhibits gasdermin D, such as wherein the inhibitor neutralizes gasdermin D.

The invention provides an antigen binding protein that binds to gasdermin D and neutralizes gasdermin D.

In some embodiments, the antigen binding protein binds to gasdermin D and inhibits gasdermin D, wherein the inhibition of gasdermin D is the inhibition of an activity of gasdermin D, such as wherein the antigen binding protein neutralizes an activity of gasdermin D and/or the inhibition of gasdermin D is the inhibition of a function of gasdermin D, such as wherein the antigen binding protein neutralizes a function of gasdermin D.

In some embodiments, the antigen binding protein binds to gasdermin D and neutralizes gasdermin D, wherein the neutralization of gasdermin D is the neutralization of an activity of gasdermin D, and/or the neutralization of gasdermin D is the neutralization of a function of gasdermin D.

In some embodiments, the antigen binding protein is an extracellular inhibitor that binds to gasdermin D.

In some embodiments antigen binding protein binds to gasdermin D on the cell surface.

In some embodiments, the antigen binding protein binds to gasdermin D and does not cross the cell membrane, unless it is bound to gasdermin D.

In some embodiments, the antigen binding protein is a large molecule.

In some embodiments, the antigen binding protein has a molecular weight of >2 kDa, >3 kDa, >4 kDa, >5 kDa, >6 kDa, >7 kDa, >8 kDa, >9 kDa or >10 kDa.

In some embodiments, the antigen binding protein binds to the N-terminal domain of gasdermin D.

In some embodiments the antigen binding protein of the invention binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein of the invention binds to an epitope comprising SEQ ID NO: 9. In some embodiments the antigen binding protein of the invention binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein of the invention binds to SEQ ID NO: 9.

In some embodiments the antigen binding protein binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an isolated peptide of SEQ ID NO: 9.

In some embodiments, the antigen binding protein binds to gasdermin D and inhibits its association with lipids, such as wherein the antigen binding protein binds to gasdermin D and neutralizes the association of gasdermin D with lipids.

In some embodiments, the antigen binding protein binds to gasdermin D and inhibits the association of gasdermin D with phosphatidylinositol 4-phosphate and/or phosphatidylinositol 4,5-bisphosphate, such as wherein the antigen binding protein binds to gasdermin D and neutralizes the association of gasdermin D with phosphatidylinositol 4-phosphate and/or phosphatidylinositol 4,5-bisphosphate.

In some embodiments, the antigen binding protein binds to gasdermin D and inhibits oligomerisation of gasdermin D, such as wherein the antigen binding protein neutralizes oligomerisation of gasdermin D.

In some embodiments, the antigen binding protein inhibits protein-protein interactions between the gasdermin D subunits, such as wherein the antigen binding protein neutralizes protein-protein interactions between the gasdermin D subunits.

In some embodiments, the antigen binding protein binds to a gasdermin D multimeric pore.

In some embodiments, the antigen binding protein binds to a gasdermin D multimeric pore and blocks the pore. In some embodiments, the antigen binding protein binds to a gasdermin D multimeric pore and disrupts protein-protein interactions between gasdermin D subunits of the pore.

In some embodiments, the antigen binding protein binds to a gasdermin D subunit of the multimeric pore. In some embodiments, the antigen binding protein binds to a gasdermin D subunit of the multimeric pore and blocks the pore.

In some embodiments, the antigen binding protein binds to a gasdermin D subunit of the multimeric pore, and disrupts the protein-protein interactions between the subunits of the pore.

In some embodiments the antigen binding protein of the invention can inhibit cell death induced by pyroptosis, such as wherein the antigen binding protein neutralizes cell death induced by pyroptosis.

In some embodiments the antigen binding protein of the invention inhibits release of IL-1β and/or IL-18, such as wherein the antigen binding protein neutralizes release of IL-1β and/or IL-18.

In some embodiments, the antigen binding protein can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein.

In some embodiments, the antigen binding protein inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl (for example 50,000 cells or 65,000 cells in 100 μl), wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antigen binding protein can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein.

In some embodiments, the antigen binding protein delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl (for example 50,000 cells or 65,000 cells in 100 μl), wherein the THP-1 cells have been pre-treated with phorbol 12-myristate 13-acetate for 16 hours, and wherein the THP-1 cells have been induced with 20 μM nigericin.

In some embodiments the antigen binding protein cross reacts with an old world monkey gasdermin D or a new world monkey gasdermin D. The advantages of such cross-reactivity are discussed above herein.

In some embodiments the antigen binding protein is an antibody or antigen-binding fragment thereof, a nanobody (VHH) domain, an IgNAR variable domain (vNAR), a Fc fusion protein, a variable lymphocyte receptor (VLR) domain, a Fibronectin type III domain, a centyrin, a Kringle domain protein, a designed ankyrin repeat protein (DARPin), a cystine-knot miniprotein, a Sso7d protein, an affibody, an affimer, an anticalin, an affilin, an affitin, or a fynomer.

Gasdermin D Specific Antigen Binding Proteins that Bind to an Epitope of Gasdermin D and Inhibit Association with Lipids

In some embodiments, the antigen binding protein binds to the N-terminal domain of gasdermin D, wherein the antigen binding protein binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an epitope comprising SEQ ID NO: 9 and wherein the antigen binding protein binds to gasdermin D and inhibits its association with lipids.

In some embodiments, the antigen binding protein binds to the N-terminal domain of gasdermin D, wherein the antigen binding protein binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an epitope comprising SEQ ID NO: 9 and wherein the antigen binding protein binds to gasdermin D and inhibits its association with phosphatidylinositol 4-phosphate and/or phosphatidylinositol 4,5-bisphosphate.

In some embodiments, the antigen binding protein binds to the N-terminal domain of gasdermin D, wherein the antigen binding protein binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an epitope comprising SEQ ID NO: 9, wherein the antigen binding protein binds to gasdermin D and inhibits its association with lipids, wherein the antigen binding protein can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antigen binding protein inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antigen binding protein binds to the N-terminal domain of gasdermin D, wherein the antigen binding protein binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an epitope comprising SEQ ID NO: 9, wherein the antigen binding protein binds to gasdermin D and inhibits its association with phosphatidylinositol 4-phosphate and/or phosphatidylinositol 4,5-bisphosphate, wherein the antigen binding protein can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antigen binding protein inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antigen binding protein binds to the N-terminal domain of gasdermin D, wherein the antigen binding protein binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an epitope comprising SEQ ID NO: 9, wherein the antigen binding protein binds to gasdermin D and inhibits its association with lipids, and wherein the antigen binding protein can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the antigen binding protein delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

In some embodiments, the antigen binding protein binds to the N-terminal domain of gasdermin D, wherein the antigen binding protein binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an epitope comprising SEQ ID NO: 9, wherein the antigen binding protein binds to gasdermin D and inhibits its association with phosphatidylinositol 4-phosphate and/or phosphatidylinositol 4,5-bisphosphate, and wherein the antigen binding protein can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the antigen binding protein delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

Gasdermin D Specific Antigen Binding Proteins that Bind to a Sequence of Gasdermin D and Inhibit Association with Lipids

In some embodiments, the antigen binding protein binds to the N-terminal domain of gasdermin D, wherein the antigen binding protein binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to SEQ ID NO: 9, and wherein the antigen binding protein binds to gasdermin D and inhibits its association with lipids.

In some embodiments, the antigen binding protein binds to the N-terminal domain of gasdermin D, wherein the antigen binding protein binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to SEQ ID NO: 9, and wherein the antigen binding protein binds to gasdermin D and inhibits its association with phosphatidylinositol 4-phosphate and/or phosphatidylinositol 4,5-bisphosphate.

In some embodiments, the antigen binding protein binds to the N-terminal domain of gasdermin D, wherein the antigen binding protein binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to SEQ ID NO: 9, wherein the antigen binding protein binds to gasdermin D and inhibits its association with lipids, and wherein the antigen binding protein can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antigen binding protein inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antigen binding protein binds to the N-terminal domain of gasdermin D, wherein the antigen binding protein binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to SEQ ID NO: 9, wherein the antigen binding protein binds to gasdermin D and inhibits its association with phosphatidylinositol 4-phosphate and/or phosphatidylinositol 4,5-bisphosphate, and wherein the antigen binding protein can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antigen binding protein inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antigen binding protein binds to the N-terminal domain of gasdermin D, wherein the antigen binding protein binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to SEQ ID NO: 9, wherein the antigen binding protein binds to gasdermin D and inhibits its association with lipids, and wherein the antigen binding protein can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the antigen binding protein delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

In some embodiments, the antigen binding protein binds to the N-terminal domain of gasdermin D, wherein the antigen binding protein binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to SEQ ID NO: 9, wherein the antigen binding protein binds to gasdermin D and inhibits its association with phosphatidylinositol 4-phosphate and/or phosphatidylinositol 4,5-bisphosphate, and wherein the antigen binding protein can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the antigen binding protein delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

Gasdermin D Specific Antigen Binding Proteins that Bind to an Isolated Peptide of Gasdermin D and Inhibit Association with Lipids

In some embodiments, the antigen binding protein binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to SEQ ID NO: 9, and wherein the antigen binding protein binds to gasdermin D and inhibits its association with lipids.

In some embodiments, the antigen binding protein binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an isolated peptide of SEQ ID NO: 9, and wherein the antigen binding protein binds to gasdermin D and inhibits its association with phosphatidylinositol 4-phosphate and/or phosphatidylinositol 4,5-bisphosphate.

In some embodiments, the antigen binding protein binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an isolated peptide of SEQ ID NO: 9, wherein the antigen binding protein binds to gasdermin D and inhibits its association with lipids, and wherein the antigen binding protein can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antigen binding protein inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antigen binding protein binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an isolated peptide of SEQ ID NO: 9, wherein the antigen binding protein binds to gasdermin D and inhibits its association with phosphatidylinositol 4-phosphate and/or phosphatidylinositol 4,5-bisphosphate, and wherein the antigen binding protein can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antigen binding protein inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antigen binding protein binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an isolated peptide of SEQ ID NO: 9, wherein the antigen binding protein binds to gasdermin D and inhibits its association with lipids, and wherein the antigen binding protein can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the antigen binding protein delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

In some embodiments, the antigen binding protein binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an isolated peptide of SEQ ID NO: 9, wherein the antigen binding protein binds to gasdermin D and inhibits its association with phosphatidylinositol 4-phosphate and/or phosphatidylinositol 4,5-bisphosphate, and wherein the antigen binding protein can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the antigen binding protein delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

Gasdermin D Specific Antigen Binding Proteins that Bind to an Epitope of Gasdermin D and Inhibit Oligomerisation

In some embodiments, the antigen binding protein binds to the N-terminal domain of gasdermin D, wherein the antigen binding protein binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an epitope comprising SEQ ID NO: 9, and wherein the antigen binding protein inhibits oligomerisation of gasdermin D.

In some embodiments, the antigen binding protein binds to the N-terminal domain of gasdermin D, wherein the antigen binding protein binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an epitope comprising SEQ ID NO: 9, and wherein the antigen binding protein inhibits protein-protein interactions between the gasdermin D subunits, thereby preventing oligomerisation to form a multimeric pore.

In some embodiments, the antigen binding protein binds to the N-terminal domain of gasdermin D, wherein the antigen binding protein binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an epitope comprising SEQ ID NO: 9, wherein the antigen binding protein inhibits oligomerisation of gasdermin D, and wherein the antigen binding protein can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antigen binding protein inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antigen binding protein binds to the N-terminal domain of gasdermin D, wherein the antigen binding protein binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an epitope comprising SEQ ID NO: 9, wherein the antigen binding protein inhibits protein-protein interactions between the gasdermin D subunits, thereby preventing oligomerisation to form a multimeric pore, and wherein the antigen binding protein can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antigen binding protein inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antigen binding protein binds to the N-terminal domain of gasdermin D, wherein the antigen binding protein binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an epitope comprising SEQ ID NO: 9, wherein the antigen binding protein inhibits oligomerisation of gasdermin D and prevents formation of the pore, and wherein the antigen binding protein can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the antigen binding protein delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

In some embodiments, the antigen binding protein binds to the N-terminal domain of gasdermin D, wherein the antigen binding protein binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an epitope comprising SEQ ID NO: 9, wherein the antigen binding protein inhibits protein-protein interactions between the gasdermin D subunits, thereby preventing oligomerisation to form a multimeric pore, and wherein the antigen binding protein can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the antigen binding protein delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

Gasdermin D Specific Antigen Binding Proteins that Bind to a Sequence of Gasdermin D and Inhibit Oligomerisation

In some embodiments, the antigen binding protein binds to the N-terminal domain of gasdermin D, wherein the antigen binding protein binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to SEQ ID NO: 9, and wherein the antigen binding protein inhibits oligomerisation of gasdermin D.

In some embodiments, the antigen binding protein binds to the N-terminal domain of gasdermin D, wherein the antigen binding protein binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to SEQ ID NO: 9, and wherein the antigen binding protein inhibits protein-protein interactions between the gasdermin D subunits, thereby preventing oligomerisation to form a multimeric pore.

In some embodiments, the antigen binding protein binds to the N-terminal domain of gasdermin D, wherein the antigen binding protein binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to SEQ ID NO: 9, wherein the antigen binding protein inhibits oligomerisation of gasdermin D, and wherein the antigen binding protein can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antigen binding protein inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antigen binding protein binds to the N-terminal domain of gasdermin D, wherein the antigen binding protein binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to SEQ ID NO: 9, wherein the antigen binding protein inhibits protein-protein interactions between the gasdermin D subunits, thereby preventing oligomerisation to form a multimeric pore, and wherein the antigen binding protein can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antigen binding protein inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antigen binding protein binds to the N-terminal domain of gasdermin D, wherein the antigen binding protein binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to SEQ ID NO: 9, wherein the antigen binding protein inhibits oligomerisation of gasdermin D, and wherein the antigen binding protein can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the antigen binding protein delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

In some embodiments, the antigen binding protein binds to the N-terminal domain of gasdermin D, wherein the antigen binding protein binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to SEQ ID NO: 9, wherein the antigen binding protein inhibits protein-protein interactions between the gasdermin D subunits, thereby preventing oligomerisation to form a multimeric pore, and wherein the antigen binding protein can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the antigen binding protein delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

Gasdermin D Specific Antigen Binding Proteins that Bind to an Isolated Peptide of Gasdermin D and Inhibit Oligomerisation

In some embodiments, the antigen binding protein binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an isolated peptide of SEQ ID NO: 9, and wherein the antigen binding protein inhibits oligomerisation of gasdermin D.

In some embodiments, the antigen binding protein binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an isolated peptide of SEQ ID NO: 9, and wherein the antigen binding protein inhibits protein-protein interactions between the gasdermin D subunits, thereby preventing oligomerisation to form a multimeric pore.

In some embodiments, the antigen binding protein binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an isolated peptide of SEQ ID NO: 9, wherein the antigen binding protein inhibits oligomerisation of gasdermin D, and wherein the antigen binding protein can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antigen binding protein inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antigen binding protein binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an isolated peptide of SEQ ID NO: 9, wherein the antigen binding protein inhibits protein-protein interactions between the gasdermin D subunits, thereby preventing oligomerisation to form a multimeric pore, and wherein the antigen binding protein can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antigen binding protein inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antigen binding protein binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an isolated peptide of SEQ ID NO: 9, wherein the antigen binding protein inhibits oligomerisation of gasdermin D, and wherein the antigen binding protein can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the antigen binding protein delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

In some embodiments, the antigen binding protein binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an isolated peptide of SEQ ID NO: 9, wherein the antigen binding protein inhibits protein-protein interactions between the gasdermin D subunits, thereby preventing oligomerisation to form a multimeric pore, and wherein the antigen binding protein can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the antigen binding protein delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

Gasdermin D Specific Antigen Binding Proteins that Bind to an Epitope of Gasdermin D and Inhibit a Gasdermin D Multimeric Pore

In some embodiments, the antigen binding protein binds to the N-terminal domain of gasdermin D, wherein the antigen binding protein binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an epitope comprising SEQ ID NO: 9, and wherein the antigen binding protein binds to a gasdermin D multimeric pore and blocks the pore.

In some embodiments, the antigen binding protein binds to the N-terminal domain of gasdermin D, wherein the antigen binding protein binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an epitope comprising SEQ ID NO: 9, wherein the antigen binding protein binds to a gasdermin D multimeric pore and blocks the pore and wherein the antigen binding protein can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antigen binding protein inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antigen binding protein binds to the N-terminal domain of gasdermin D, wherein the antigen binding protein binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an epitope comprising SEQ ID NO: 9, wherein the antigen binding protein binds to a gasdermin D multimeric pore and blocks the pore, and wherein the antigen binding protein can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the antigen binding protein delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

In some embodiments, the antigen binding protein binds to the N-terminal domain of gasdermin D, wherein the antigen binding protein binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an epitope comprising SEQ ID NO: 9, wherein the antigen binding protein binds to a gasdermin D multimeric pore, and disrupts the protein-protein interactions between the subunits of the pore.

In some embodiments, the antigen binding protein binds to the N-terminal domain of gasdermin D, wherein the antigen binding protein binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an epitope comprising SEQ ID NO: 9, wherein the antigen binding protein binds to a gasdermin D multimeric pore, and disrupts the protein-protein interactions between the subunits of the pore and wherein the antigen binding protein can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antigen binding protein inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antigen binding protein binds to the N-terminal domain of gasdermin D, wherein the antigen binding protein binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an epitope comprising SEQ ID NO: 9, wherein the antigen binding protein binds to a gasdermin D multimeric pore, and disrupts the protein-protein interactions between the subunits of the pore, and wherein the antigen binding protein can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the antigen binding protein delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

Gasdermin D Specific Antigen Binding Proteins that Bind to an Epitope of a Gasdermin D Subunit of the Multimeric Pore, and Inhibit a Gasdermin D Multimeric Pore

In some embodiments, the antigen binding protein binds to the N-terminal domain of gasdermin D, wherein the antigen binding protein binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an epitope comprising SEQ ID NO: 9, and wherein the antigen binding protein binds to a gasdermin D subunit of the multimeric pore and blocks the pore.

In some embodiments, the antigen binding protein binds to the N-terminal domain of gasdermin D, wherein the antigen binding protein binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an epitope comprising SEQ ID NO: 9, wherein the antigen binding protein binds to a gasdermin D subunit of the multimeric pore and blocks the pore, and wherein the antigen binding protein can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antigen binding protein inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antigen binding protein binds to the N-terminal domain of gasdermin D, wherein the antigen binding protein binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an epitope comprising SEQ ID NO: 9, wherein the antigen binding protein binds to a gasdermin D subunit of the multimeric pore and blocks the pore, and wherein the antigen binding protein can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the antigen binding protein delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

In some embodiments, the antigen binding protein binds to the N-terminal domain of gasdermin D, wherein the antigen binding protein binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an epitope comprising SEQ ID NO: 9, and wherein the antigen binding protein binds to a gasdermin D subunit of the multimeric pore, and disrupts the protein-protein interactions between the subunits of the pore.

In some embodiments, the antigen binding protein binds to the N-terminal domain of gasdermin D, wherein the antigen binding protein binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an epitope comprising SEQ ID NO: 9, wherein the antigen binding protein binds to a gasdermin D subunit of the multimeric pore, and disrupts the protein-protein interactions between the subunits of the pore and wherein the antigen binding protein can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, optionally wherein the antigen binding protein inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antigen binding protein binds to the N-terminal domain of gasdermin D, wherein the antigen binding protein binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an epitope comprising SEQ ID NO: 9, wherein the antigen binding protein binds to a gasdermin D subunit of the multimeric pore, and disrupts the protein-protein interactions between the subunits of the pore, and wherein the antigen binding protein can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the antigen binding protein delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

Gasdermin D Specific Antigen Binding Proteins that Bind to a Sequence of Gasdermin D and Inhibit a Gasdermin D Multimeric Pore

In some embodiments, the antigen binding protein binds to the N-terminal domain of gasdermin D, wherein the antigen binding protein binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to SEQ ID NO: 9, and wherein the antigen binding protein binds to a gasdermin D multimeric pore and blocks the pore.

In some embodiments, the antigen binding protein binds to the N-terminal domain of gasdermin D, wherein the antigen binding protein binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to SEQ ID NO: 9, wherein the antigen binding protein binds to a gasdermin D multimeric pore and blocks the pore and wherein the antigen binding protein can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antigen binding protein inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antigen binding protein binds to the N-terminal domain of gasdermin D, wherein the antigen binding protein binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to SEQ ID NO: 9, wherein the antigen binding protein binds to a gasdermin D multimeric pore and blocks the pore, and wherein the antigen binding protein can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the antigen binding protein delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

In some embodiments, the antigen binding protein binds to the N-terminal domain of gasdermin D, wherein the antigen binding protein binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to SEQ ID NO: 9, and wherein the antigen binding protein binds to a gasdermin D multimeric pore, and disrupts the protein-protein interactions between the subunits of the pore.

In some embodiments, the antigen binding protein binds to the N-terminal domain of gasdermin D, wherein the antigen binding protein binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to SEQ ID NO: 9, wherein the antigen binding protein binds to a gasdermin D multimeric pore, and disrupts the protein-protein interactions between the subunits of the pore and wherein the antigen binding protein can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antigen binding protein inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antigen binding protein binds to the N-terminal domain of gasdermin D, wherein the antigen binding protein binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to SEQ ID NO: 9, wherein the antigen binding protein binds to a gasdermin D multimeric pore, and disrupts the protein-protein interactions between the subunits of the pore, and wherein the antigen binding protein can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the antigen binding protein delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

Gasdermin D Specific Antigen Binding Proteins that Bind to a Sequence of a Gasdermin D Subunit of the Multimeric Pore, and Inhibit a Gasdermin D Multimeric Pore

In some embodiments, the antigen binding protein binds to the N-terminal domain of gasdermin D, wherein the antigen binding protein binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to SEQ ID NO: 9, and wherein the antigen binding protein binds to a gasdermin D subunit of the multimeric pore and blocks the pore.

In some embodiments, the antigen binding protein binds to the N-terminal domain of gasdermin D, wherein the antigen binding protein binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to SEQ ID NO: 9, wherein the antigen binding protein binds to a gasdermin D subunit of the multimeric pore and blocks the pore and wherein the antigen binding protein can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antigen binding protein inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antigen binding protein binds to the N-terminal domain of gasdermin D, wherein the antigen binding protein binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to SEQ ID NO: 9, wherein the antigen binding protein binds to a gasdermin D subunit of the multimeric pore and blocks the pore, and wherein the antigen binding protein can delay cell death induced by pyroptosis by >0.1 hours, >0.2 hours, >0.5 hours, >0.75 hours, >1 hour, >2 hours, >3 hours, >4 hours, or >6 hours, optionally wherein the antigen binding protein delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, for example in a THP-1 cell assay as described herein, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

In some embodiments, the antigen binding protein binds to the N-terminal domain of gasdermin D, wherein the antigen binding protein binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to SEQ ID NO: 9, and wherein the antigen binding protein binds to a gasdermin D subunit of the multimeric pore, and disrupts the protein-protein interactions between the subunits of the pore.

In some embodiments, the antigen binding protein binds to the N-terminal domain of gasdermin D, wherein the antigen binding protein binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to SEQ ID NO: 9, wherein the antigen binding protein binds to a gasdermin D subunit of the multimeric pore, and disrupts the protein-protein interactions between the subunits of the pore and wherein the antigen binding protein can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antigen binding protein inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antigen binding protein binds to the N-terminal domain of gasdermin D, wherein the antigen binding protein binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to SEQ ID NO: 9, wherein the antigen binding protein binds to a gasdermin D subunit of the multimeric pore, and disrupts the protein-protein interactions between the subunits of the pore, and wherein the antigen binding protein can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the antigen binding protein delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

Gasdermin D Specific Antigen Binding Proteins that Bind to an Isolated Peptide of Gasdermin D and Inhibit a Gasdermin D Multimeric Pore

In some embodiments, the antigen binding protein binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an isolated peptide of SEQ ID NO: 9, and wherein the antigen binding protein binds to a gasdermin D multimeric pore and blocks the pore.

In some embodiments, the antigen binding protein binds to the N-terminal domain of gasdermin D and binds to an isolated peptide of o SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an isolated peptide of SEQ ID NO: 9, wherein the antigen binding protein binds to a gasdermin D multimeric pore and blocks the pore and wherein the antigen binding protein can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antigen binding protein inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antigen binding protein binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an isolated peptide of SEQ ID NO: 9, wherein the antigen binding protein binds to a gasdermin D multimeric pore and blocks the pore, and wherein the antigen binding protein can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the antigen binding protein delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

In some embodiments, the antigen binding protein binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an isolated peptide of SEQ ID NO: 9, and wherein the antigen binding protein binds to a gasdermin D multimeric pore, and disrupts the protein-protein interactions between the subunits of the pore.

In some embodiments, the antigen binding protein binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an isolated peptide of SEQ ID NO: 9, wherein the antigen binding protein binds to a gasdermin D multimeric pore, and disrupts the protein-protein interactions between the subunits of the pore and wherein the antigen binding protein can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antigen binding protein inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antigen binding protein binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an isolated peptide of SEQ ID NO: 9, wherein the antigen binding protein binds to a gasdermin D multimeric pore, and disrupts the protein-protein interactions between the subunits of the pore, and wherein the antigen binding protein can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the antigen binding protein delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

Gasdermin D Specific Antigen Binding Proteins that Bind to an Isolated Peptide of a Gasdermin D Subunit of the Multimeric Pore, and Inhibit a Gasdermin D Multimeric Pore

In some embodiments, the antigen binding protein binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an isolated peptide of SEQ ID NO: 9, and wherein the antigen binding protein binds to a gasdermin D subunit of the multimeric pore and blocks the pore.

In some embodiments, the antigen binding protein binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an isolated peptide of SEQ ID NO: 9, wherein the antigen binding protein binds to a gasdermin D subunit of the multimeric pore and blocks the pore and wherein the antigen binding protein can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antigen binding protein inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antigen binding protein binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an isolated peptide of SEQ ID NO: 9, wherein the antigen binding protein binds to a gasdermin D subunit of the multimeric pore and blocks the pore, and wherein the antigen binding protein can delay cell death induced by pyroptosis by >0.1 hours, >0.2 hours, >0.5 hours, >0.75 hours, >1 hour, >2 hours, >3 hours, >4 hours, or >6 hours, optionally wherein the antigen binding protein delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, for example in a THP-1 cell assay as described herein, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

In some embodiments, the antigen binding protein binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an isolated peptide of SEQ ID NO: 9, and wherein the antigen binding protein binds to a gasdermin D subunit of the multimeric pore, and disrupts the protein-protein interactions between the subunits of the pore.

In some embodiments, the antigen binding protein binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an isolated peptide of SEQ ID NO: 9, wherein the antigen binding protein binds to a gasdermin D subunit of the multimeric pore, and disrupts the protein-protein interactions between the subunits of the pore and wherein the antigen binding protein can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antigen binding protein inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antigen binding protein binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antigen binding protein binds to an isolated peptide of SEQ ID NO: 9, wherein the antigen binding protein binds to a gasdermin D subunit of the multimeric pore, and disrupts the protein-protein interactions between the subunits of the pore, and wherein the antigen binding protein can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the antigen binding protein delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

### Gasdermin D Specific Antibodies or Antigen Binding Fragments

Described herein are antibodies or antigen-binding fragments that specifically bind gasdermin D. The general structure of an antibody molecule comprises an antigen binding domain, and the Fc domain. The Fc mediates effector functions. Naturally occurring antibody structural units typically comprise a tetramer of 2 light and 2 heavy chains. The antigen binding domain is formed of a variable domain from the heavy chain and a variable domain from the light chain. The variable regions typically exhibit the same general structure of relatively conserved framework regions (FR) joined by three hyper variable regions, also called complementarity determining regions or CDRs. The CDRs from the two chains of each pair typically are aligned by the framework regions, which can enable binding to a specific epitope. From N-terminal to C-terminal, both light and heavy chain variable regions typically comprise the domains FR1, CDR1, FR2, CDR2, FR3, CDR3 and FR4. The variable region of an antibody typically determines specificity of a particular antibody for its target. Human light chains are typically classified as kappa and lambda light chains. Heavy chains are typically classified as mu, delta, gamma, alpha, or epsilon, and define the antibody's isotype as IgM, IgD, IgG, IgA, and IgE, respectively. IgG has several subclasses, including, but not limited to, IgG1, IgG2, IgG3, and IgG4. IgM has subclasses too. A full-length light chain includes a variable region domain, VL, and a constant region domain, CL—The variable region domain of the light chain is at the amino-terminus of the polypeptide. Light chains include kappa chains and lambda chains. The term “heavy chain” includes a full-length heavy chain and fragments thereof having sufficient variable region sequence to confer binding specificity. A full-length heavy chain includes a variable region domain, VH, and three constant region domains, CH1, CH2, and CH3. The VH domain is at the amino-terminus of the polypeptide, and the CH domains are at the carboxyl-terminus, with the CH3 being closest to the carboxy-terminus of the polypeptide. “Antibody” refers to all isotypes of immunoglobulins (IgG, IgA, IgE, IgM, IgD, and IgY) including various monomeric, polymeric and chimeric forms, unless otherwise specified. Specifically encompassed by the term “antibody” are polyclonal antibodies, monoclonal antibodies (mAbs), and antibody-like polypeptides, such as chimeric antibodies and humanized antibodies.

In some embodiments, the antigen binding protein is an antibody or antigen binding fragment thereof.

The invention provides an antibody or antigen binding fragment thereof that binds to gasdermin D and inhibits gasdermin D, such as wherein the antibody or antigen binding fragment thereof neutralizes gasdermin D.

The invention provides an antibody or antigen binding fragment thereof that binds to gasdermin D and neutralizes gasdermin D.

In some embodiments, the antibody or antigen binding fragment thereof binds to gasdermin D and inhibits gasdermin D, wherein the inhibition of gasdermin D is the inhibition of an activity of gasdermin D, such as wherein the antibody or antigen binding fragment neutralizes an activity of gasdermin D and/or the inhibition of gasdermin D is the inhibition of a function of gasdermin D, such as wherein the antibody or antigen binding fragment neutralizes a function of gasdermin D.

In some embodiments, the antibody or antigen binding fragment thereof binds to gasdermin D and neutralizes gasdermin D, wherein the neutralization of gasdermin D is the neutralization of an activity of gasdermin D, and/or the neutralization of gasdermin D is the neutralization of a function of gasdermin D.

In some embodiments, the antibody or antigen binding fragment thereof is an extracellular inhibitor that binds to gasdermin D.

In some embodiments, the antibody or antigen binding fragment thereof binds to gasdermin D on the cell surface.

In some embodiments, the antibody or antigen binding fragment thereof binds to gasdermin D and does not cross the cell membrane, unless it is bound to gasdermin D.

In some embodiments, the antibody or antigen binding fragment thereof is a large molecule.

In some embodiments, the antibody or antigen binding fragment thereof has a molecular weight of >2 kDa, >3 kDa, >4 kDa, >5 kDa, >6 kDa, >7 kDa, >8 kDa, >9 kDa or >10 kDa.

In some embodiments, the antibody or antigen binding fragment thereof binds to the N-terminal domain of gasdermin D.

In some embodiments the antibody or antigen binding fragment thereof of the invention binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof of the invention binds to an epitope comprising SEQ ID NO: 9. In some embodiments the antibody or antigen binding fragment thereof of the invention binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof of the invention binds to SEQ ID NO: 9.

In some embodiments, the antibody or antigen binding fragment thereof binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an isolated peptide of SEQ ID NO: 9.

In some embodiments, the antibody or antigen binding fragment thereof binds to gasdermin D and inhibits its association with lipids, such as wherein the antibody or antigen binding fragment thereof binds to gasdermin D and neutralizes the association of gasdermin D with lipids.

In some embodiments, the antibody or antigen binding fragment thereof binds to gasdermin D and inhibits the association of gasdermin D with phosphatidylinositol 4-phosphate and/or phosphatidylinositol 4,5-bisphosphate, such as wherein the antibody or antigen binding fragment thereof binds to gasdermin D and neutralizes the association of gasdermin D with phosphatidylinositol 4-phosphate and/or phosphatidylinositol 4,5-bisphosphate.

In some embodiments, the antibody or antigen binding fragment thereof binds to gasdermin D and inhibits oligomerisation of gasdermin D, such as wherein the antibody or antigen binding fragment thereof neutralizes oligomerisation of gasdermin D.

In some embodiments, the antibody or antigen binding fragment thereof inhibits protein-protein interactions between the gasdermin D subunits, such as wherein the antibody or antigen binding fragment thereof neutralizes protein-protein interactions between the gasdermin D subunits.

In some embodiments, the antibody or antigen binding fragment thereof binds to a gasdermin D multimeric pore.

In some embodiments, the antibody or antigen binding fragment thereof binds to a gasdermin D multimeric pore and blocks the pore. In some embodiments, the antibody or antigen binding fragment thereof binds to a gasdermin D multimeric pore and disrupts protein-protein interactions between gasdermin D subunits of the pore.

In some embodiments, the antibody or antigen binding fragment thereof binds to a gasdermin D subunit of the multimeric pore. In some embodiments, the antibody or antigen binding fragment thereof binds to a gasdermin D subunit of the multimeric pore and blocks the pore.

In some embodiments, the antibody or antigen binding fragment thereof binds to a gasdermin D subunit of the multimeric pore, and disrupts the protein-protein interactions between the subunits of the pore.

In some embodiments the antibody or antigen binding fragment thereof can inhibit cell death induced by pyroptosis, such as wherein the antibody or antigen binding fragment thereof neutralizes cell death induced by pyroptosis.

In some embodiments the antibody or antigen binding fragment thereof inhibits release of IL-1β and/or IL-18, such as wherein the antibody or antigen binding fragment neutralizes release of IL-1β and/or IL-18.

In some embodiments, the antibody or antigen binding fragment thereof can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein.

In some embodiments, the antibody or antigen binding fragment thereof inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl (for example 50,000 cell or 65,000 cells in 100 μl), wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antibody or antigen binding fragment thereof can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein.

In some embodiments, the antibody or antigen binding fragment thereof delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl (for example 50,000 cell or 65,000 cells in 100 μl), wherein the THP-1 cells have been pre-treated with phorbol 12-myristate 13-acetate for 16 hours, and wherein the THP-1 cells have been induced with 20 μM nigericin.

In some embodiments the antibody or antigen binding fragment thereof cross reacts with an old world monkey gasdermin D or a new world monkey gasdermin D. The advantages of such cross-reactivity are discussed above herein.

Gasdermin D Specific Antibodies or Antigen Binding Fragments Thereof that Bind to an Epitope of Gasdermin D and Inhibit Association with Lipids

In some embodiments, the antibody or antigen binding fragment thereof binds to the N-terminal domain of gasdermin D, wherein the antibody or antigen binding fragment thereof binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an epitope comprising SEQ ID NO: 9 and wherein the antibody or antigen binding fragment thereof binds to gasdermin D and inhibits its association with lipids.

In some embodiments, the antibody or antigen binding fragment thereof binds to the N-terminal domain of gasdermin D, wherein the antibody or antigen binding fragment thereof binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an epitope comprising SEQ ID NO: 9 and wherein the antibody or antigen binding fragment thereof binds to gasdermin D and inhibits its association with phosphatidylinositol 4-phosphate and/or phosphatidylinositol 4,5-bisphosphate.

In some embodiments, the antibody or antigen binding fragment thereof binds to the N-terminal domain of gasdermin D, wherein the antibody or antigen binding fragment thereof binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an epitope comprising SEQ ID NO: 9, wherein the antibody or antigen binding fragment thereof binds to gasdermin D and inhibits its association with lipids, and wherein the antibody or antigen binding fragment thereof can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antibody or antigen binding fragment thereof inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antibody or antigen binding fragment thereof binds to the N-terminal domain of gasdermin D, wherein the antibody or antigen binding fragment thereof binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an epitope comprising SEQ ID NO: 9, wherein the antibody or antigen binding fragment thereof binds to gasdermin D and inhibits its association with phosphatidylinositol 4-phosphate and/or phosphatidylinositol 4,5-bisphosphate, and wherein the antibody or antigen binding fragment thereof can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antibody or antigen binding fragment thereof inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antibody or antigen binding fragment thereof binds to the N-terminal domain of gasdermin D, wherein the antibody or antigen binding fragment thereof binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an epitope comprising SEQ ID NO: 9, wherein the antibody or antigen binding fragment thereof binds to gasdermin D and inhibits its association with lipids, and wherein the antibody or antigen binding fragment thereof can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the antibody or antigen binding fragment thereof delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

In some embodiments, the antibody or antigen binding fragment thereof binds to the N-terminal domain of gasdermin D, wherein the antibody or antigen binding fragment thereof binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an epitope comprising SEQ ID NO: 9, wherein the antibody or antigen binding fragment thereof binds to gasdermin D and inhibits its association with phosphatidylinositol 4-phosphate and/or phosphatidylinositol 4,5-bisphosphate, and wherein the antibody or antigen binding fragment thereof can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the antibody or antigen binding fragment thereof delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

Gasdermin D Specific Antibodies or Antigen Binding Fragments Thereof that Bind to a Sequence of Gasdermin D and Inhibit Association with Lipids

In some embodiments, the antibody or antigen binding fragment thereof binds to the N-terminal domain of gasdermin D, wherein the antibody or antigen binding fragment thereof binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to SEQ ID NO: 9, and wherein the antibody or antigen binding fragment thereof binds to gasdermin D and inhibits its association with lipids.

In some embodiments, the antibody or antigen binding fragment thereof binds to the N-terminal domain of gasdermin D, wherein the antibody or antigen binding fragment thereof binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to SEQ ID NO: 9, and wherein the antibody or antigen binding fragment thereof binds to gasdermin D and inhibits its association with phosphatidylinositol 4-phosphate and/or phosphatidylinositol 4,5-bisphosphate.

In some embodiments, the antibody or antigen binding fragment thereof binds to the N-terminal domain of gasdermin D, wherein the antibody or antigen binding fragment thereof binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to SEQ ID NO: 9, wherein the antibody or antigen binding fragment thereof binds to gasdermin D and inhibits its association with lipids, and wherein the antibody or antigen binding fragment thereof can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antibody or antigen binding fragment thereof inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antibody or antigen binding fragment thereof binds to the N-terminal domain of gasdermin D, wherein the antibody or antigen binding fragment thereof binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to SEQ ID NO: 9, wherein the antibody or antigen binding fragment thereof binds to gasdermin D and inhibits its association with phosphatidylinositol 4-phosphate and/or phosphatidylinositol 4,5-bisphosphate, and wherein the antibody or antigen binding fragment thereof can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antibody or antigen binding fragment thereof inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antibody or antigen binding fragment thereof binds to the N-terminal domain of gasdermin D, wherein the antibody or antigen binding fragment thereof binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to SEQ ID NO: 9, wherein the antibody or antigen binding fragment thereof binds to gasdermin D and inhibits its association with lipids, and wherein the antibody or antigen binding fragment thereof can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the antibody or antigen binding fragment thereof delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

In some embodiments, the antibody or antigen binding fragment thereof binds to the N-terminal domain of gasdermin D, wherein the antibody or antigen binding fragment thereof binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to SEQ ID NO: 9, wherein the antibody or antigen binding fragment thereof binds to gasdermin D and inhibits its association with phosphatidylinositol 4-phosphate and/or phosphatidylinositol 4,5-bisphosphate, and wherein the antibody or antigen binding fragment thereof can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the antibody or antigen binding fragment thereof delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

Gasdermin D Specific Antibodies or Antigen Binding Fragments Thereof that Bind to an Isolated Peptide of Gasdermin D and Inhibit Association with Lipids

In some embodiments, the antibody or antigen binding fragment thereof binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to SEQ ID NO: 9, and wherein the antibody or antigen binding fragment thereof binds to gasdermin D and inhibits its association with lipids.

In some embodiments, the antibody or antigen binding fragment thereof binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an isolated peptide of SEQ ID NO: 9, and wherein the antibody or antigen binding fragment thereof binds to gasdermin D and inhibits its association with phosphatidylinositol 4-phosphate and/or phosphatidylinositol 4,5-bisphosphate.

In some embodiments, the antibody or antigen binding fragment thereof binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an isolated peptide of SEQ ID NO: 9, wherein the antibody or antigen binding fragment thereof binds to gasdermin D and inhibits its association with lipids, and wherein the antibody or antigen binding fragment thereof can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antibody or antigen binding fragment thereof inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antibody or antigen binding fragment thereof binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an isolated peptide of SEQ ID NO: 9, wherein the antibody or antigen binding fragment thereof binds to gasdermin D and inhibits its association with phosphatidylinositol 4-phosphate and/or phosphatidylinositol 4,5-bisphosphate, and wherein the antibody or antigen binding fragment thereof can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antibody or antigen binding fragment thereof inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antibody or antigen binding fragment thereof binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an isolated peptide of SEQ ID NO: 9, wherein the antibody or antigen binding fragment thereof binds to gasdermin D and inhibits its association with lipids, and wherein the antibody or antigen binding fragment thereof can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the antibody or antigen binding fragment thereof delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

In some embodiments, the antibody or antigen binding fragment thereof binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an isolated peptide of SEQ ID NO: 9, wherein the antibody or antigen binding fragment thereof binds to gasdermin D and inhibits its association with phosphatidylinositol 4-phosphate and/or phosphatidylinositol 4,5-bisphosphate, and wherein the antibody or antigen binding fragment thereof can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the antibody or antigen binding fragment thereof delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

Gasdermin D Specific Antibodies or Antigen Binding Fragments Thereof that Bind to an Epitope of Gasdermin D and Inhibit Oligomerisation

In some embodiments, the antibody or antigen binding fragment thereof binds to the N-terminal domain of gasdermin D, wherein the antibody or antigen binding fragment thereof binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an epitope comprising SEQ ID NO: 9, and wherein the antibody or antigen binding fragment thereof inhibits oligomerisation of gasdermin D.

In some embodiments, the antibody or antigen binding fragment thereof binds to the N-terminal domain of gasdermin D, wherein the antibody or antigen binding fragment thereof binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an epitope comprising SEQ ID NO: 9, and wherein the antibody or antigen binding fragment thereof inhibits protein-protein interactions between the gasdermin D subunits, thereby preventing oligomerisation to form a multimeric pore.

In some embodiments, the antibody or antigen binding fragment thereof binds to the N-terminal domain of gasdermin D, wherein the antibody or antigen binding fragment thereof binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an epitope comprising SEQ ID NO: 9, wherein the antibody or antigen binding fragment thereof inhibits oligomerisation of gasdermin D, and wherein the antibody or antigen binding fragment thereof can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antibody or antigen binding fragment thereof inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antibody or antigen binding fragment thereof binds to the N-terminal domain of gasdermin D, wherein the antibody or antigen binding fragment thereof binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an epitope comprising SEQ ID NO: 9, wherein the antibody or antigen binding fragment thereof inhibits protein-protein interactions between the gasdermin D subunits, thereby preventing oligomerisation to form a multimeric pore, and wherein the antibody or antigen binding fragment thereof can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antibody or antigen binding fragment thereof inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antibody or antigen binding fragment thereof binds to the N-terminal domain of gasdermin D, wherein the antibody or antigen binding fragment thereof binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an epitope comprising SEQ ID NO: 9, wherein the antibody or antigen binding fragment thereof inhibits oligomerisation of gasdermin D and prevents formation of the pore, and wherein the antibody or antigen binding fragment thereof can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the antibody or antigen binding fragment thereof delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

In some embodiments, the antibody or antigen binding fragment thereof binds to the N-terminal domain of gasdermin D, wherein the antibody or antigen binding fragment thereof binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an epitope comprising SEQ ID NO: 9, wherein the antibody or antigen binding fragment thereof inhibits protein-protein interactions between the gasdermin D subunits, thereby preventing oligomerisation to form a multimeric pore, and wherein the antibody or antigen binding fragment thereof can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the antibody or antigen binding fragment thereof delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

Gasdermin D Specific Antibodies or Antigen Binding Fragments Thereof that Bind to a Sequence of Gasdermin D and Inhibit Oligomerisation

In some embodiments, the antibody or antigen binding fragment thereof binds to the N-terminal domain of gasdermin D, wherein the antibody or antigen binding fragment thereof binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to SEQ ID NO: 9, and wherein the antibody or antigen binding fragment thereof inhibits oligomerisation of gasdermin D.

In some embodiments, the antibody or antigen binding fragment thereof binds to the N-terminal domain of gasdermin D, wherein the antibody or antigen binding fragment thereof binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to SEQ ID NO: 9, and wherein the antibody or antigen binding fragment thereof inhibits protein-protein interactions between the gasdermin D subunits, thereby preventing oligomerisation to form a multimeric pore.

In some embodiments, the antibody or antigen binding fragment thereof binds to the N-terminal domain of gasdermin D, wherein the antibody or antigen binding fragment thereof binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to SEQ ID NO: 9, wherein the antibody or antigen binding fragment thereof inhibits oligomerisation of gasdermin D, and wherein the antibody or antigen binding fragment thereof can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antibody or antigen binding fragment thereof inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antibody or antigen binding fragment thereof binds to the N-terminal domain of gasdermin D, wherein the antibody or antigen binding fragment thereof binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to SEQ ID NO: 9, wherein the antibody or antigen binding fragment thereof inhibits protein-protein interactions between the gasdermin D subunits, thereby preventing oligomerisation to form a multimeric pore, and wherein the antibody or antigen binding fragment thereof can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antibody or antigen binding fragment thereof inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antibody or antigen binding fragment thereof binds to the N-terminal domain of gasdermin D, wherein the antibody or antigen binding fragment thereof binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to SEQ ID NO: 9, wherein the antibody or antigen binding fragment thereof inhibits oligomerisation of gasdermin D, and wherein the antibody or antigen binding fragment thereof can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the antibody or antigen binding fragment thereof delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

In some embodiments, the antibody or antigen binding fragment thereof binds to the N-terminal domain of gasdermin D, wherein the antibody or antigen binding fragment thereof binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to SEQ ID NO: 9, wherein the antibody or antigen binding fragment thereof inhibits protein-protein interactions between the gasdermin D subunits, thereby preventing oligomerisation to form a multimeric pore, and wherein the antibody or antigen binding fragment thereof can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the antibody or antigen binding fragment thereof delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

Gasdermin D Specific Antibodies or Antigen Binding Fragments Thereof that Bind to an Isolated Peptide of Gasdermin D and Inhibit Oligomerisation

In some embodiments, the antibody or antigen binding fragment thereof binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an isolated peptide of SEQ ID NO: 9, and wherein the antibody or antigen binding fragment thereof inhibits oligomerisation of gasdermin D.

In some embodiments, the antibody or antigen binding fragment thereof binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an isolated peptide of SEQ ID NO: 9, and wherein the antibody or antigen binding fragment thereof inhibits protein-protein interactions between the gasdermin D subunits, thereby preventing oligomerisation to form a multimeric pore.

In some embodiments, the antibody or antigen binding fragment thereof binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an isolated peptide of SEQ ID NO: 9, wherein the antibody or antigen binding fragment thereof inhibits oligomerisation of gasdermin D, and wherein the antibody or antigen binding fragment thereof can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antibody or antigen binding fragment thereof inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antibody or antigen binding fragment thereof binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an isolated peptide of SEQ ID NO: 9, wherein the antibody or antigen binding fragment thereof inhibits protein-protein interactions between the gasdermin D subunits, thereby preventing oligomerisation to form a multimeric pore, and wherein the antibody or antigen binding fragment thereof can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antibody or antigen binding fragment thereof inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antibody or antigen binding fragment thereof binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an isolated peptide of SEQ ID NO: 9, wherein the antibody or antigen binding fragment thereof inhibits oligomerisation of gasdermin D, and wherein the antibody or antigen binding fragment thereof can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the antibody or antigen binding fragment thereof delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

In some embodiments, the antibody or antigen binding fragment thereof binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an isolated peptide of SEQ ID NO: 9, wherein the antibody or antigen binding fragment thereof inhibits protein-protein interactions between the gasdermin D subunits, thereby preventing oligomerisation to form a multimeric pore, and wherein the antibody or antigen binding fragment thereof can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the antibody or antigen binding fragment thereof delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

Gasdermin D Specific Antibodies or Antigen Binding Fragments Thereof that Bind to an Epitope of Gasdermin D and Inhibit a Gasdermin D Multimeric Pore

In some embodiments, the antibody or antigen binding fragment thereof binds to the N-terminal domain of gasdermin D, wherein the antibody or antigen binding fragment thereof binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an epitope comprising SEQ ID NO: 9, and wherein the antibody or antigen binding fragment thereof binds to a gasdermin D multimeric pore and blocks the pore.

In some embodiments, the antibody or antigen binding fragment thereof binds to the N-terminal domain of gasdermin D, wherein the antibody or antigen binding fragment thereof binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an epitope comprising SEQ ID NO: 9, wherein the antibody or antigen binding fragment thereof binds to a gasdermin D multimeric pore and blocks the pore and wherein the antibody or antigen binding fragment thereof can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antibody or antigen binding fragment thereof inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antibody or antigen binding fragment thereof binds to the N-terminal domain of gasdermin D, wherein the antibody or antigen binding fragment thereof binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an epitope comprising SEQ ID NO: 9, wherein the antibody or antigen binding fragment thereof binds to a gasdermin D multimeric pore and blocks the pore, and wherein the antibody or antigen binding fragment thereof can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the antibody or antigen binding fragment thereof delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

In some embodiments, the antibody or antigen binding fragment thereof binds to the N-terminal domain of gasdermin D, wherein the antibody or antigen binding fragment thereof binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an epitope comprising SEQ ID NO: 9, wherein the antibody or antigen binding fragment thereof binds to a gasdermin D multimeric pore, and disrupts the protein-protein interactions between the subunits of the pore.

In some embodiments, the antibody or antigen binding fragment thereof binds to the N-terminal domain of gasdermin D, wherein the antibody or antigen binding fragment thereof binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an epitope comprising SEQ ID NO: 9, wherein the antibody or antigen binding fragment thereof binds to a gasdermin D multimeric pore, and disrupts the protein-protein interactions between the subunits of the pore and wherein the antibody or antigen binding fragment thereof can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antibody or antigen binding fragment thereof inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antibody or antigen binding fragment thereof binds to the N-terminal domain of gasdermin D, wherein the antibody or antigen binding fragment thereof binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an epitope comprising SEQ ID NO: 9, wherein the antibody or antigen binding fragment thereof binds to a gasdermin D multimeric pore, and disrupts the protein-protein interactions between the subunits of the pore, and wherein the antibody or antigen binding fragment thereof can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the antibody or antigen binding fragment thereof delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

Gasdermin D Specific Antibodies or Antigen Binding Fragments Thereof that Bind to an Epitope of a Gasdermin D Subunit of the Multimeric Pore, and Inhibit a Gasdermin D Multimeric Pore

In some embodiments, the antibody or antigen binding fragment thereof binds to the N-terminal domain of gasdermin D, wherein the antibody or antigen binding fragment thereof binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an epitope comprising SEQ ID NO: 9, and wherein the antibody or antigen binding fragment thereof binds to a gasdermin D subunit of the multimeric pore and blocks the pore.

In some embodiments, the antibody or antigen binding fragment thereof binds to the N-terminal domain of gasdermin D, wherein the antibody or antigen binding fragment thereof binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an epitope comprising SEQ ID NO: 9, wherein the antibody or antigen binding fragment thereof binds to a gasdermin D subunit of the multimeric pore and blocks the pore, and wherein the antibody or antigen binding fragment thereof can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antibody or antigen binding fragment thereof inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antibody or antigen binding fragment thereof binds to the N-terminal domain of gasdermin D, wherein the antibody or antigen binding fragment thereof binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an epitope comprising SEQ ID NO: 9, wherein the antibody or antigen binding fragment thereof binds to a gasdermin D subunit of the multimeric pore and blocks the pore, and wherein the antibody or antigen binding fragment thereof can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the antibody or antigen binding fragment thereof delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

In some embodiments, the antibody or antigen binding fragment thereof binds to the N-terminal domain of gasdermin D, wherein the antibody or antigen binding fragment thereof binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an epitope comprising SEQ ID NO: 9, and wherein the antibody or antigen binding fragment thereof binds to a gasdermin D subunit of the multimeric pore, and disrupts the protein-protein interactions between the subunits of the pore.

In some embodiments, the antibody or antigen binding fragment thereof binds to the N-terminal domain of gasdermin D, wherein the antibody or antigen binding fragment thereof binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an epitope comprising SEQ ID NO: 9, wherein the antibody or antigen binding fragment thereof binds to a gasdermin D subunit of the multimeric pore, and disrupts the protein-protein interactions between the subunits of the pore and wherein the antibody or antigen binding fragment thereof can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, optionally wherein the antibody or antigen binding fragment thereof inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antibody or antigen binding fragment thereof binds to the N-terminal domain of gasdermin D, wherein the antibody or antigen binding fragment thereof binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an epitope comprising SEQ ID NO: 9, wherein the antibody or antigen binding fragment thereof binds to a gasdermin D subunit of the multimeric pore, and disrupts the protein-protein interactions between the subunits of the pore, and wherein the antibody or antigen binding fragment thereof can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the antibody or antigen binding fragment thereof delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

Gasdermin D Specific Antibodies or Antigen Binding Fragments Thereof that Bind to a Sequence of Gasdermin D and Inhibit a Gasdermin D Multimeric Pore

In some embodiments, the antibody or antigen binding fragment thereof binds to the N-terminal domain of gasdermin D, wherein the antibody or antigen binding fragment thereof binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to SEQ ID NO: 9, and wherein the antibody or antigen binding fragment thereof binds to a gasdermin D multimeric pore and blocks the pore.

In some embodiments, the antibody or antigen binding fragment thereof binds to the N-terminal domain of gasdermin D, wherein the antibody or antigen binding fragment thereof binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to SEQ ID NO: 9, wherein the antibody or antigen binding fragment thereof binds to a gasdermin D multimeric pore and blocks the pore and wherein the antibody or antigen binding fragment thereof can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antibody or antigen binding fragment thereof inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antibody or antigen binding fragment thereof binds to the N-terminal domain of gasdermin D, wherein the antibody or antigen binding fragment thereof binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to SEQ ID NO: 9, wherein the antibody or antigen binding fragment thereof binds to a gasdermin D multimeric pore and blocks the pore, and wherein the antibody or antigen binding fragment thereof can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the antibody or antigen binding fragment thereof delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

In some embodiments, the antibody or antigen binding fragment thereof binds to the N-terminal domain of gasdermin D, wherein the antibody or antigen binding fragment thereof binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to SEQ ID NO: 9, and wherein the antibody or antigen binding fragment thereof binds to a gasdermin D multimeric pore, and disrupts the protein-protein interactions between the subunits of the pore.

In some embodiments, the antibody or antigen binding fragment thereof binds to the N-terminal domain of gasdermin D, wherein the antibody or antigen binding fragment thereof binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to SEQ ID NO: 9, wherein the antibody or antigen binding fragment thereof binds to a gasdermin D multimeric pore, and disrupts the protein-protein interactions between the subunits of the pore and wherein the antibody or antigen binding fragment thereof can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antibody or antigen binding fragment thereof inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antibody or antigen binding fragment thereof binds to the N-terminal domain of gasdermin D, wherein the antibody or antigen binding fragment thereof binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to SEQ ID NO: 9, wherein the antibody or antigen binding fragment thereof binds to a gasdermin D multimeric pore, and disrupts the protein-protein interactions between the subunits of the pore, and wherein the antibody or antigen binding fragment thereof can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the antibody or antigen binding fragment thereof delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

Gasdermin D Specific Antibodies or Antigen Binding Fragments Thereof that Bind to a Sequence of a Gasdermin D Subunit of the Multimeric Pore, and Inhibit a Gasdermin D Multimeric Pore

In some embodiments, the antibody or antigen binding fragment thereof binds to the N-terminal domain of gasdermin D, wherein the antibody or antigen binding fragment thereof binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to SEQ ID NO: 9, and wherein the antibody or antigen binding fragment thereof binds to a gasdermin D subunit of the multimeric pore and blocks the pore.

In some embodiments, the antibody or antigen binding fragment thereof binds to the N-terminal domain of gasdermin D, wherein the antibody or antigen binding fragment thereof binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to SEQ ID NO: 9, wherein the antibody or antigen binding fragment thereof binds to a gasdermin D subunit of the multimeric pore and blocks the pore and wherein the antibody or antigen binding fragment thereof can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antibody or antigen binding fragment thereof inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antibody or antigen binding fragment thereof binds to the N-terminal domain of gasdermin D, wherein the antibody or antigen binding fragment thereof binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to SEQ ID NO: 9, wherein the antibody or antigen binding fragment thereof binds to a gasdermin D subunit of the multimeric pore and blocks the pore, and wherein the antibody or antigen binding fragment thereof can delay cell death induced by pyroptosis by >0.1 hours, >0.2 hours, >0.5 hours, >0.75 hours, >1 hour, >2 hours, >3 hours, >4 hours, or >6 hours, optionally wherein the antibody or antigen binding fragment thereof delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, for example in a THP-1 cell assay as described herein, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

In some embodiments, the antibody or antigen binding fragment thereof binds to the N-terminal domain of gasdermin D, wherein the antibody or antigen binding fragment thereof binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to SEQ ID NO: 9, and wherein the antibody or antigen binding fragment thereof binds to a gasdermin D subunit of the multimeric pore, and disrupts the protein-protein interactions between the subunits of the pore.

In some embodiments, the antibody or antigen binding fragment thereof binds to the N-terminal domain of gasdermin D, wherein the antibody or antigen binding fragment thereof binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to SEQ ID NO: 9, wherein the antibody or antigen binding fragment thereof binds to a gasdermin D subunit of the multimeric pore, and disrupts the protein-protein interactions between the subunits of the pore and wherein the antibody or antigen binding fragment thereof can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antibody or antigen binding fragment thereof inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antibody or antigen binding fragment thereof binds to the N-terminal domain of gasdermin D, wherein the antibody or antigen binding fragment thereof binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to SEQ ID NO: 9, wherein the antibody or antigen binding fragment thereof binds to a gasdermin D subunit of the multimeric pore, and disrupts the protein-protein interactions between the subunits of the pore, and wherein the antibody or antigen binding fragment thereof can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the antibody or antigen binding fragment thereof delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

Gasdermin D Specific Antibodies and Antigen Binding Fragments Thereof that Bind to an Isolated Peptide of Gasdermin D and Inhibit a Gasdermin D Multimeric Pore

In some embodiments, the antibody or antigen binding fragment thereof binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an isolated peptide of SEQ ID NO: 9, and wherein the antibody or antigen binding fragment thereof binds to a gasdermin D multimeric pore and blocks the pore.

In some embodiments, the antibody or antigen binding fragment thereof binds to the N-terminal domain of gasdermin D and binds to an isolated peptide of o SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an isolated peptide of SEQ ID NO: 9, wherein the antibody or antigen binding fragment thereof binds to a gasdermin D multimeric pore and blocks the pore and wherein the antibody or antigen binding fragment thereof can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antibody or antigen binding fragment thereof inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antibody or antigen binding fragment thereof binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an isolated peptide of SEQ ID NO: 9, wherein the antibody or antigen binding fragment thereof binds to a gasdermin D multimeric pore and blocks the pore, and wherein the antibody or antigen binding fragment thereof can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the antibody or antigen binding fragment thereof delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

In some embodiments, the antibody or antigen binding fragment thereof binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an isolated peptide of SEQ ID NO: 9, and wherein the antibody or antigen binding fragment thereof binds to a gasdermin D multimeric pore, and disrupts the protein-protein interactions between the subunits of the pore.

In some embodiments, the antibody or antigen binding fragment thereof binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an isolated peptide of SEQ ID NO: 9, wherein the antibody or antigen binding fragment thereof binds to a gasdermin D multimeric pore, and disrupts the protein-protein interactions between the subunits of the pore and wherein the antibody or antigen binding fragment thereof can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antibody or antigen binding fragment thereof inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antibody or antigen binding fragment thereof binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an isolated peptide of SEQ ID NO: 9, wherein the antibody or antigen binding fragment thereof binds to a gasdermin D multimeric pore, and disrupts the protein-protein interactions between the subunits of the pore, and wherein the antibody or antigen binding fragment thereof can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the antibody or antigen binding fragment thereof delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

Gasdermin D Specific Antibodies or Antigen Binding Fragments Thereof that Bind to an Isolated Peptide of a Gasdermin D Subunit of the Multimeric Pore, and Inhibit a Gasdermin D Multimeric Pore

In some embodiments, the antibody or antigen binding fragment thereof binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an isolated peptide of SEQ ID NO: 9, and wherein the antibody or antigen binding fragment thereof binds to a gasdermin D subunit of the multimeric pore and blocks the pore.

In some embodiments, the antibody or antigen binding fragment thereof binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an isolated peptide of SEQ ID NO: 9, wherein the antibody or antigen binding fragment thereof binds to a gasdermin D subunit of the multimeric pore and blocks the pore and wherein the antibody or antigen binding fragment thereof can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antibody or antigen binding fragment thereof inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antibody or antigen binding fragment thereof binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an isolated peptide of SEQ ID NO: 9, wherein the antibody or antigen binding fragment thereof binds to a gasdermin D subunit of the multimeric pore and blocks the pore, and wherein the antibody or antigen binding fragment thereof can delay cell death induced by pyroptosis by >0.1 hours, >0.2 hours, >0.5 hours, >0.75 hours, >1 hour, >2 hours, >3 hours, >4 hours, or >6 hours, optionally wherein the antibody or antigen binding fragment thereof delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, for example in a THP-1 cell assay as described herein, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

In some embodiments, the antibody or antigen binding fragment thereof binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an isolated peptide of SEQ ID NO: 9, and wherein the antibody or antigen binding fragment thereof binds to a gasdermin D subunit of the multimeric pore, and disrupts the protein-protein interactions between the subunits of the pore.

In some embodiments, the antibody or antigen binding fragment thereof binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an isolated peptide of SEQ ID NO: 9, wherein the antibody or antigen binding fragment thereof binds to a gasdermin D subunit of the multimeric pore, and disrupts the protein-protein interactions between the subunits of the pore and wherein the antibody or antigen binding fragment thereof can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the antibody or antigen binding fragment thereof inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the antibody or antigen binding fragment thereof binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the antibody or antigen binding fragment thereof binds to an isolated peptide of SEQ ID NO: 9, wherein the antibody or antigen binding fragment thereof binds to a gasdermin D subunit of the multimeric pore, and disrupts the protein-protein interactions between the subunits of the pore, and wherein the antibody or antigen binding fragment thereof can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the antibody or antigen binding fragment thereof delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

In some embodiments, the antibody or antigen binding fragment thereof comprises an Fc region. An “Fc” region comprises two heavy chain fragments comprising the C_{H}1 and C_{H}2 domains of an antibody. The two heavy chain fragments are held together by two or more disulfide bonds and by hydrophobic interactions of the C_{H}3 domains.

In some embodiments the antibody or antigen binding fragment comprises a IgA, IgD, IgE, IgG or IgM isotype. The described gasdermin D-specific antibodies or antigen-binding fragments include all isotypes, IgA, IgD, IgE, IgG and IgM, and synthetic multimers of the four-chain immunoglobulin structure. The described antibodies or antigen-binding fragments also include the IgY isotype generally found in hen or turkey serum and hen or turkey egg yolk.

The gasdermin D-specific antibodies and antigen-binding fragments may be derived by recombinant means. For use in administration to humans, non-human derived antibodies or antigen-binding fragments may be genetically or structurally altered to be less antigenic upon administration to a human patient.

The IgG class is divided in four isotypes: IgG1, IgG2, IgG3 and IgG4 in humans. They share more than 95% homology in the amino acid sequences of the Fc regions but show major differences in the amino acid composition and structure of the hinge region. The Fc region mediates effector functions, such as antibody-dependent cellular cytotoxicity (ADCC) and complement-dependent cytotoxicity (CDC). In ADCC, the Fc region of an antibody binds to Fc receptors (FcgRs) on the surface of immune effector cells such as natural killers and macrophages, leading to the phagocytosis or lysis of the targeted cells. In CDC, the antibodies kill the targeted cells by triggering the complement cascade at the cell surface. The antibodies described herein include antibodies with the described features of the variable domains in combination with any of the IgG isotypes, including modified versions in which the Fc sequence has been modified to effect different effector functions.

For many applications of therapeutic antibodies, Fc-mediated effector functions are not part of the mechanism of action. These Fc-mediated effector functions can be detrimental and potentially pose a safety risk by causing off-mechanism toxicity. Modifying effector functions can be achieved by engineering the Fc regions to reduce their binding to FcgRs or the complement factors. The binding of IgG to the activating (FcgRI, FcgRIIa, FcgRIIIa and FcgRIIIb) and inhibitory (FcgRIIb) FcgRs or the first component of complement (Clq) depends on residues located in the hinge region and the CH2 domain. Mutations have been introduced in IgG1, IgG2 and IgG4 to reduce or silence Fc functionalities. The antibodies described herein may include these modifications.

In one embodiment, the antibody comprises an Fc region with one or more of the following properties: (a) reduced effector function when compared to the parent Fc; (b) reduced affinity to Fcg RI, Fcg RIIa, Fcg RIIb, Fcg RIIIb and/or Fcg RIIIa, (c) reduced affinity to FcgRI (d) reduced affinity to FcgRIIa (e) reduced affinity to FcgRIIb, (f) reduced affinity to Fcg RIIIb or (g) reduced affinity to FcgRIIIa.

In some embodiments, the antibodies or antigen-binding fragments are IgG, or derivatives thereof, e.g., IgG1, IgG2, IgG3, and IgG4 isotypes. In some embodiments the antibody is an IgG1 isotype. In some embodiments wherein the antibody has an IgG1 isotype, the antibody contains L234A, L235A, and/or K409R substitution(s) in its Fc region. In some embodiments the antibody is an IgG4 isotype. In some embodiments wherein the antibody has an IgG4 isotype, the antibody contains S228P, L234A, and L235A substitutions in its Fc region. The antibodies described herein may include these modifications.

Antibody or antigen-binding fragment thereof specificity is largely determined by the amino acid sequence, and arrangement, of the CDRs. Therefore, the CDRs of one isotype may be transferred to another isotype without altering antigen specificity. Alternatively, techniques have been established to cause hybridomas to switch from producing one antibody isotype to another (isotype switching) without altering antigen specificity. Accordingly, such antibody isotypes are within the scope of the described antibodies or antigen-binding fragments.

In some embodiments, the antibodies or antigen-binding fragments are chimeric. As used herein, the term “chimeric” refers to an antibody, or antigen-binding fragment thereof, having at least some portion of at least one variable domain derived from the antibody amino acid sequence of a non-human mammal, a rodent, or a reptile, while the remaining portions of the antibody, or antigen-binding fragment thereof, are derived from a human.

In some embodiments, the antibodies are humanized antibodies. Humanized antibodies may be chimeric immunoglobulins, immunoglobulin chains or fragments thereof (such as Fv, Fab, Fab′, F(ab′)2 or other antigen-binding subsequences of antibodies) that contain minimal sequence derived from non-human immunoglobulin. For the most part, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin sequence. The humanized antibody may include at least a portion of an immunoglobulin constant region (Fc), typically that of a human immunoglobulin. A “humanized” antibody has a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant domains of the heavy and/or light chains of the non-human species antibody are mutated to produce the humanized antibody. In another embodiment, the constant domain(s) from a human antibody are fused to the variable domain(s) of a non-human species. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen.

In certain embodiments, a humanized antibody is substantially non-immunogenic in humans. In certain embodiments, a humanized antibody has substantially the same affinity for a target as an antibody from another species from which the humanized antibody is derived. In certain embodiments, modification of an antibody by methods known in the art is typically designed to achieve increased binding affinity for a target and/or to reduce immunogenicity of the antibody in the recipient. In certain embodiments, humanized antibodies are modified to eliminate glycosylation sites in order to increase affinity of the antibody for its cognate antigen. In certain such embodiments, such techniques typically reduce antibody immunogenicity by reducing the number of foreign residues, but do not prevent anti-idiotypic and anti-allotypic responses following repeated administration of the antibodies.

In some embodiments, the antibody or antigen-binding fragment thereof can comprise a human. Human antibodies can be made in rodents which have had their Ig genes deleted or inactivated and replaces with human Ig genes. Large human Ig fragments can preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains can yield high affinity fully human antibodies against any antigen of interest, including human antigens. Human antibodies avoid some of the problems associated with antibodies that possess murine or rat variable and/or constant regions. The presence of such murine or rat derived proteins can lead to the rapid clearance of the antibodies or can lead to the generation of an immune response against the antibody by a patient. In order to avoid the utilization of murine or rat derived antibodies, fully human antibodies can be generated through the introduction of functional human antibody loci into a rodent, other mammal or animal so that the rodent, other mammal or animal produces fully human antibodies. Humanized antibodies are those antibodies that, while initially starting off containing antibody amino acid sequences that are not human, have had at least some of these nonhuman antibody amino acid sequences replaced with human antibody sequences. This is in contrast with human antibodies, in which the antibody is encoded (or capable of being encoded) by genes possessed a human.

In some embodiments the antigen binding protein is an antigen binding fragment. In some embodiments the antigen-binding fragment includes Fab fragments, F(ab′)2 fragments, Fv fragments, or single-chain Fv (scFv) molecules. The phrase “an antibody or antigen binding fragment thereof” may be used to denote that a given antigen binding fragment incorporates one or more amino acid segments of the antibody referred to in the phrase. Antigen-binding fragments include those provided by any known technique, such as enzymatic cleavage, peptide synthesis, and recombinant techniques. Some antigen-binding fragments are composed of portions of intact antibodies that retain antigen-binding specificity of the parent antibody molecule. For example, antigen-binding fragments may comprise at least one variable region (either a heavy chain or light chain variable region) or one or more CDRs of an antibody known to bind a particular antigen. Examples of suitable antigen-binding fragments include, without limitation diabodies and single-chain molecules as well as Fab, F(ab′)2, Fc, Fabc, and Fv molecules, single chain (Sc) antibodies, individual antibody light chains, individual antibody heavy chains, chimeric fusions between antibody chains or CDRs and other proteins, heavy chain monomers or dimers, light chain monomers or dimers, dimers consisting of one heavy and one light chain, a monovalent fragment consisting of the VL, VH, CL and CH1 domains, or a monovalent antibody as described in WO2007059782, bivalent fragments comprising two Fab fragments linked by a disulfide bridge at the hinge region, a Fd fragment consisting essentially of the VH and CH1 domains; a Fv fragment consisting essentially of the VL and VH domains of a single arm of an antibody, a dAb fragment (Ward et al., Nature 341, 544-546 (1989)), which consists essentially of a VH domain and also called domain antibodies (Holt et al; Trends Biotechnol. 2003 November; 21(11):484-90); an isolated complementarity determining region (CDR), and the like. Antigen binding fragments may be recombinantly produced or produced by enzymatic or chemical cleavage of intact antibodies.

A “Fab fragment” comprises one light chain and the C_{H}1 and variable regions of one heavy chain. The heavy chain of a Fab molecule cannot form a disulfide bond with another heavy chain molecule.

A “Fab′ fragment” comprises one light chain and a portion of one heavy chain that contains the V_{H} domain and the C_{H}1 domain and also the region between the C_{H}1 and C_{H}2 domains, such that an interchain disulfide bond can be formed between the two heavy chains of two Fab′ fragments to form an F(ab′)2 molecule.

A “F(ab′)₂ fragment” contains two light chains and two heavy chains containing a portion of the constant region between the C_{H}1 and C_{H}2 domains, such that an interchain disulfide bond is formed between the two heavy chains.

A F(ab′)₂ fragment thus is composed of two Fab′ fragments that are held together by a disulfide bond between the two heavy chains.

The “Fv region” comprises the variable regions from both the heavy and light chains, but lacks the constant regions.

“Single-chain antibodies” are Fv molecules in which the heavy and light chain variable regions have been connected by a flexible linker to form a single polypeptide chain, which forms an antigen binding region.

Also disclosed are isolated polynucleotides that encode the antibodies or antigen-binding fragments that immunospecifically bind to gasdermin D. The isolated polynucleotides capable of encoding the variable domain segments provided herein may be included on the same, or different, vectors to produce antibodies or antigen-binding fragments.

Polynucleotides encoding recombinant antigen-binding proteins also are within the scope of the disclosure. In some embodiments, the polynucleotides described (and the peptides they encode) include a leader sequence. Any leader sequence known in the art may be employed. The leader sequence may include, but is not limited to, a restriction site or a translation start site.

The gasdermin D-specific antibodies or antigen-binding fragments described herein include variants having single or multiple amino acid substitutions, deletions, or additions that retain the biological properties (e.g., binding affinity or immune effector activity) of the described gasdermin D-specific antibodies or antigen-binding fragments. In the context of the present invention the following notations are, unless otherwise indicated, used to describe a mutation; i) substitution of an amino acid in a given position is written as e.g. L234A which means a substitution of a leucine in position 234 with an alanine; and ii) for specific variants the specific three or one letter codes are used, including the codes Xaa and X to indicate any amino acid residue. Thus, the substitution of leucine for alanine in position 234 is designated as: L234A, or the substitution of leucine for any amino acid residue in position 234 is designated as L234X. In case of deletion of leucine in position 234 it is indicated by L234*. The skilled person may produce variants having single or multiple amino acid substitutions, deletions, or additions.

These variants may include: (a) variants in which one or more amino acid residues are substituted with conservative or nonconservative amino acids, (b) variants in which one or more amino acids are added to or deleted from the polypeptide, (c) variants in which one or more amino acids include a substituent group, and (d) variants in which the polypeptide is fused with another peptide or polypeptide such as a fusion partner, a protein tag or other chemical moiety, that may confer useful properties to the polypeptide, such as, for example, an epitope for an antibody, a polyhistidine sequence, a biotin moiety and the like. Antibodies or antigen-binding fragments described herein may include variants in which amino acid residues from one species are substituted for the corresponding residue in another species, either at the conserved or nonconserved positions. In other embodiments, amino acid residues at nonconserved positions are substituted with conservative or nonconservative residues. The techniques for obtaining these variants, including genetic (deletions, mutations, etc.), chemical, and enzymatic techniques, are known to persons having ordinary skill in the art.

The gasdermin D-specific antibodies or antigen-binding fragments described herein have binding affinities for gasdermin D that include a dissociation constant (K_{D}) of less than about 5×10⁻⁷ M, preferably less than about 5×10⁻⁸ M. In some embodiments, the gasdermin D-specific antibodies or antigen-binding fragments described herein have binding affinities for gasdermin D that include a dissociation constant (K_{D}) of less than about 5×10⁻⁷ M, preferably less than about 5×10⁻⁸ M. The affinity of the described gasdermin D-specific antibodies, or antigen-binding fragments, may be determined by a variety of methods known in the art, such as surface plasmon resonance or ELISA-based methods. Assays for measuring affinity by SPR include assays performed using a BIAcore 3000 machine, where the assay is performed at room temperature (e.g. at or near 25° C.), wherein the antibody capable of binding to gasdermin D is captured on the BIAcore sensor chip by an anti-Fc antibody (e.g. goat anti-human IgG Fc specific antibody Jackson ImmunoResearch laboratories Prod #109-005-098) to a level around 75 RUs, followed by the collection of association and dissociation data at a flow rate of 40 μl/min. “Specific binding” or “immunospecific binding” or derivatives thereof when used in the context of antibodies, or antibody fragments, represents binding via domains encoded by immunoglobulin genes or fragments of immunoglobulin genes to one or more epitopes of a protein of interest, without preferentially binding other molecules in a sample containing a mixed population of molecules. Typically, an antibody binds to a cognate antigen with a K_{D} of less than about 1×10⁻⁸M, as measured by a surface plasmon resonance assay or a cell binding assay. Phrases such as “[antigen]-specific” antibody (e.g., gasdermin D-specific antibody) are meant to convey that the recited antibody specifically binds the recited antigen.

The term “k_{d}” (sec⁻¹), as used herein, refers to the dissociation rate constant of a particular antibody-antigen interaction. Said value is also referred to as the koff value.

The term “kₐ” (M⁻¹ sec⁻¹), as used herein, refers to the association rate constant of a particular antibody-antigen interaction.

The term “K_{D}” (M), as used herein, refers to the dissociation equilibrium constant of a particular antibody-antigen interaction.

The term “K_{A}” (M⁻¹), as used herein, refers to the association equilibrium constant of a particular antibody-antigen interaction and is obtained by dividing the kₐ by the k_{d}.

When used herein in the context of two or more antibodies or antigen-binding fragments, the term “competes with” or “cross-competes with” indicates that the two or more antibodies or antigen-binding fragments compete for binding to gasdermin D. Unless otherwise defined or negated by context, the terms “competes with” or “cross-competes with” when used herein is also intended to cover such pairs of antibodies or antigen-binding fragments.

Also provided are vectors comprising the polynucleotides described herein. The vectors can be expression vectors. Recombinant expression vectors containing a sequence encoding a polypeptide of interest are thus contemplated as within the scope of this disclosure. The expression vector may contain one or more additional sequences such as but not limited to regulatory sequences (e.g., promoter, enhancer), a selection marker, and a polyadenylation signal. Vectors for transforming a wide variety of host cells are well known and include, but are not limited to, plasmids, phagemids, cosmids, baculoviruses, bacmids, bacterial artificial chromosomes (BACs), yeast artificial chromosomes (YACs), as well as other bacterial, yeast and viral vectors.

Recombinant expression vectors within the scope of the description include synthetic, genomic, or cDNA-derived nucleic acid fragments that encode at least one recombinant protein which may be operably linked to suitable regulatory elements. Such regulatory elements may include a transcriptional promoter, sequences encoding suitable mRNA ribosomal binding sites, and sequences that control the termination of transcription and translation. Expression vectors, especially mammalian expression vectors, may also include one or more nontranscribed elements such as an origin of replication, a suitable promoter and enhancer linked to the gene to be expressed, other 5′ or 3′ flanking nontranscribed sequences, 5′ or 3′ nontranslated sequences (such as necessary ribosome binding sites), a polyadenylation site, splice donor and acceptor sites, or transcriptional termination sequences. An origin of replication that confers the ability to replicate in a host may also be incorporated.

The transcriptional and translational control sequences in expression vectors to be used in transforming vertebrate cells may be provided by viral sources. Exemplary vectors may be constructed as described by Okayama and Berg, 3 Mol. Cell. Biol. 280 (1983).

In some embodiments, the antibody- or antigen-binding fragment-coding sequence is placed under control of a powerful constitutive promoter, such as the promoters for the following genes: hypoxanthine phosphoribosyl transferase (HPRT), adenosine deaminase, pyruvate kinase, beta-actin, human myosin, human hemoglobin, human muscle creatine, and others. In addition, many viral promoters function constitutively in eukaryotic cells and are suitable for use with the described embodiments. Such viral promoters include without limitation, Cytomegalovirus (CMV) immediate early promoter, the early and late promoters of SV40, the Mouse Mammary Tumor Virus (MMTV) promoter, the long terminal repeats (LTRs) of Maloney leukemia virus, Human Immunodeficiency Virus (HIV), Epstein Barr Virus (EBV), Rous Sarcoma Virus (RSV), and other retroviruses, and the thymidine kinase promoter of Herpes Simplex Virus. In one embodiment, the gasdermin D-specific antibody or antigen-binding fragment thereof coding sequence is placed under control of an inducible promoter such as the metallothionein promoter, tetracycline-inducible promoter, doxycycline-inducible promoter, promoters that contain one or more interferon-stimulated response elements (ISRE) such as protein kinase R 2′,5′-oligoadenylate synthetases, Mx genes, ADAR1, and the like.

Vectors described herein may contain one or more Internal Ribosome Entry Site(s) (IRES). Inclusion of an IRES sequence into fusion vectors may be beneficial for enhancing expression of some proteins. In some embodiments the vector system will include one or more polyadenylation sites (e.g., SV40), which may be upstream or downstream of any of the aforementioned nucleic acid sequences. Vector components may be contiguously linked, or arranged in a manner that provides optimal spacing for expressing the gene products (i.e., by the introduction of “spacer” nucleotides between the ORFs), or positioned in another way. Regulatory elements, such as the IRES motif, may also be arranged to provide optimal spacing for expression.

The vectors may comprise selection markers, which are well known in the art. Selection markers include positive and negative selection markers, for example, antibiotic resistance genes (e.g., neomycin resistance gene, a hygromycin resistance gene, a kanamycin resistance gene, a tetracycline resistance gene, a penicillin resistance gene), glutamate synthase genes, HSV-TK, HSV-TK derivatives for ganciclovir selection, or bacterial purine nucleoside phosphorylase gene for 6-methylpurine selection (Gadi et al., 7 Gene Ther. 1738-1743 (2000)). A nucleic acid sequence encoding a selection marker or the cloning site may be upstream or downstream of a nucleic acid sequence encoding a polypeptide of interest or cloning site.

The vectors described herein may be used to transform various cells with the genes encoding the described antibodies or antigen-binding fragments. For example, the vectors may be used to generate gasdermin D-specific antibody or antigen-binding fragment-producing cells. Thus, another aspect features host cells transformed with vectors comprising a nucleic acid sequence encoding an antibody or antigen-binding fragment thereof that specifically binds gasdermin D, such as the antibodies or antigen-binding fragments described and exemplified herein.

Numerous techniques are known in the art for the introduction of foreign genes into cells and may be used to construct the recombinant cells for purposes of carrying out the described methods, in accordance with the various embodiments described and exemplified herein. The technique used should provide for the stable transfer of the heterologous gene sequence to the host cell, such that the heterologous gene sequence is heritable and expressible by the cell progeny, and so that the necessary development and physiological functions of the recipient cells are not disrupted. Techniques which may be used include but are not limited to chromosome transfer (e.g., cell fusion, chromosome mediated gene transfer, micro cell mediated gene transfer), physical methods (e.g., transfection, spheroplast fusion, microinjection, electroporation, liposome carrier), viral vector transfer (e.g., recombinant DNA viruses, recombinant RNA viruses) and the like (described in Cline, 29 Pharmac. Ther. 69-92 (1985)). Calcium phosphate precipitation and polyethylene glycol (PEG)-induced fusion of bacterial protoplasts with mammalian cells may also be used to transform cells.

Also provided are host cells comprising the vectors described herein. Cells suitable for use in the expression of the gasdermin D-specific antibodies or antigen-binding fragments described herein are preferably eukaryotic cells, more preferably cells of plant, rodent, or human origin, for example but not limited to NSO, CHO, CHOK1, perC.6, Tk-ts13, BHK, HEK293 cells, COS-7, T98G, CV-1/EBNA, L cells, C127, 3T3, HeLa, NS1, Sp2/0 myeloma cells, and BHK cell lines, among others. In addition, expression of antibodies may be accomplished using hybridoma cells. Methods for producing hybridomas are well established in the art.

Cells transformed with expression vectors described herein may be selected or screened for recombinant expression of the antibodies or antigen-binding fragments described herein. Recombinant-positive cells are expanded and screened for subclones exhibiting a desired phenotype, such as high level expression, enhanced growth properties, or the ability to yield proteins with desired biochemical characteristics, for example, due to protein modification or altered post-translational modifications. These phenotypes may be due to inherent properties of a given subclone or to mutation. Mutations may be effected through the use of chemicals, UV-wavelength light, radiation, viruses, insertional mutagens, inhibition of DNA mismatch repair, or a combination of such methods.

Also provided are methods for producing the antibody or antigen binding fragment thereof, comprising culturing the host cell under conditions such that the antibody or antigen binding fragment thereof is produced.

### Gasdermin D Specific Multispecific Antibodies

In some embodiments the antigen binding protein is a multispecific antibody. The binding domains of the anti-gasdermin D antibodies described herein recognize cells expressing gasdermin D on their surface. More specific targeting to particular subsets of cells can be achieved by making bispecific molecules, such as antibodies or antibody fragments, which bind to gasdermin D and to another target. In addition more specific targeting to gasdermin D can be achieved by binding to multiple epitopes of gasdermin D using a molecule that recognizes multiple epitopes. This is achieved by making a molecule which comprises a first region binding to gasdermin D and a second binding region binding to the further antigen or different epitope region. The antigen-binding regions can take any form that allows specific recognition of the target, for example the binding region may be or may include a heavy chain variable domain, an Fv (combination of a heavy chain variable domain and a light chain variable domain), or any antigen binding protein as described herein. Accordingly, bispecific molecules comprising two different antigen-binding regions which bind gasdermin D and another antigen, respectively, are provided. Accordingly, bispecific molecules comprising two different antigen-binding regions, which bind to different epitopes of gasdermin D are provided.

A bispecific or bifunctional antibody typically is an artificial hybrid antibody having two different heavy/light chain pairs and two different binding sites. Bispecific antibodies can be produced by a variety of methods including, but not limited to, fusion of hybridomas or linking of Fab′ fragments. See, e.g., Songsivilai et al., Clin. Exp. Immunol., 79: 315-321 (1990); Kostelny et al., J. Immunol., 148:1547-1553 (1992).

Different formats of bispecific antibodies have been describes and were recently reviewed by Chames and Baty (2009) Curr Opin Drug Disc Dev 12: 276.

In some embodiments, the bispecific antibody of the present invention is a diabody, a cross-body, or a bispecific antibody obtained via a controlled Fab arm exchange as those described in the present invention.

In some embodiments, the bispecific antibodies include IgG-like molecules with complementary CH3 domains to force heterodimerisation; recombinant IgG-like dual targeting molecules, wherein the two sides of the molecule each contain the Fab fragment or part of the Fab fragment of at least two different antibodies; IgG fusion molecules, wherein full length IgG antibodies are fused to an extra Fab fragment or parts of Fab fragment; Fc fusion molecules, wherein single chain Fv molecules or stabilized diabodies are fused to heavy-chain constant-domains, Fc-regions or parts thereof, Fab fusion molecules, wherein different Fab-fragments are fused together; ScFv- and diabody-based and heavy chain antibodies (e.g., domain antibodies, nanobodies) wherein different single chain Fv molecules or different diabodies or different heavy-chain antibodies (e.g. domain antibodies, nanobodies) are fused to each other or to another protein or carrier molecule.

In some embodiments, IgG-like molecules with complementary CH3 domains molecules include the Triomab/Quadroma (Trion Pharma/Fresenius Biotech), the Knobs-into-Holes (Genentech), CrossMAbs (Roche) and the electrostatically-matched (Amgen), the LUZ-Y (Genentech), the Strand Exchange Engineered Domain body (SEEDbody)(EMD Serono), the Biclonic (Merus) and the DuoBody (Genmab A/S).

In some embodiments, recombinant IgG-like dual targeting molecules include Dual Targeting (DT)-Ig (GSK/Domantis), Two-in-one Antibody (Genentech), Cross-linked Mabs (Karmanos Cancer Center), mAb2 (F-Star) and CovX-body (CovX/Pfizer).

In some embodiments, IgG fusion molecules include Dual Variable Domain (DVD)-Ig (Abbott), IgG-like Bispecific (InnClone/Eli Lilly), Ts2Ab (Medlmmune/AZ) and BsAb (Zymogenetics), HERCULES (Biogen Idec) and TvAb (Roche).

In some embodiments, Fc fusion molecules include to ScFv/Fc Fusions (Academic Institution), SCORPION (Emergent BioSolutions/Trubion, Zymogenetics/BMS), Dual Affinity Retargeting Technology (Fc-DART) (MacroGenics) and Dual(ScFv).sub.2-Fab (National Research Center for Antibody Medicine—China).

In some embodiments, Fab fusion bispecific antibodies include F(ab)2 (Medarex/AMGEN), Dual-Action or Bis-Fab (Genentech), Dock-and-Lock (DNL) (ImmunoMedics), Bivalent Bispecific (Biotecnol) and Fab-Fv (UCB-Celltech). ScFv-, diabody-based and domain antibodies include but are not limited to Bispecific T Cell Engager (BITE) (Micromet), Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting nanobodies (Ablynx), dual targeting heavy chain only domain antibodies.

Full length bispecific antibodies of the invention may be generated for example using Fab arm exchange (or half molecule exchange) between two mono specific bivalent antibodies by introducing substitutions at the heavy chain CH3 interface in each half molecule to favor heterodimer formation of two antibody half molecules having distinct specificity either in vitro in cell-free environment or using co-expression. The Fab arm exchange reaction is the result of a disulfide-bond isomerization reaction and dissociation-association of CH3 domains. The heavy-chain disulfide bonds in the hinge regions of the parent mono specific antibodies are reduced. The resulting free cysteines of one of the parent monospecific antibodies form an inter heavy-chain disulfide bond with cysteine residues of a second parent mono specific antibody molecule and simultaneously CH3 domains of the parent antibodies release and reform by dissociation-association. The CH3 domains of the Fab arms may be engineered to favor heterodimerization over homodimerization. The resulting product is a bispecific antibody having two Fab arms or half molecules which each bind a distinct epitope, i.e. an epitope on gasdermin D and an epitope on another target molecule.

“Homodimerization” as used herein refers to an interaction of two heavy chains having identical CH3 amino acid sequences. “Homodimer” as used herein refers to an antibody having two heavy chains with identical CH3 amino acid sequences.

“Heterodimerization” as used herein refers to an interaction of two heavy chains having non-identical CH3 amino acid sequences. “Heterodimer” as used herein refers to an antibody having two heavy chains with non-identical CH3 amino acid sequences.

The “knob-in-hole” strategy (see, e.g., PCT Int. Publ. No. WO 2006/028936) may be used to generate full length bispecific antibodies. Briefly, selected amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob”. Exemplary CH3 substitution pairs forming a knob and a hole are (expressed as modified position in the first CH3 domain of the first heavy chain/modified position in the second CH3 domain of the second heavy chain): T366Y/F405A, T366W/F405W, F405W/Y407A, T394W/Y407T, T394S/Y407A, T366W/T394S, F405W/T394S and T366W/T366S_L368A_Y407V.

Other strategies such as promoting heavy chain heterodimerization using electrostatic interactions by substituting positively charged residues at one CH3 surface and negatively charged residues at a second CH3 surface may be used, as described in US Pat. Publ. No. US2010/0015133; US Pat. Publ. No. US2009/0182127; US Pat. Publ. No. US2010/028637 or US Pat. Publ. No. US2011/0123532. In other strategies, heterodimerization may be promoted by the following substitutions (expressed as modified position in the first CH3 domain of the first heavy chain/modified position in the second CH3 domain of the second heavy chain): L351Y_F405AY407V/T394W, T3661_K392M_T394W/F405A_Y407V, T366L_K392M_T394W/F405A_Y407V, L351Y_Y407A/T366A_K409F, L351Y_Y407A/T366V K409F Y407A/T366A_K409F, or T350V_L351Y_F405A Y407V/T350V_T366L_K392L_T394W as described in U.S. Pat. Publ. No. US2012/0149876 or U.S. Pat. Publ. No. US2013/0195849.

In addition to methods described above, bispecific antibodies of the invention may be generated in vitro in a cell-free environment by introducing asymmetrical mutations in the CH3 regions of two mono specific homodimeric antibodies and forming the bispecific heterodimeric antibody from two parent monospecific homodimeric antibodies in reducing conditions to allow disulfide bond isomerization according to methods described in Inti. Pat. Publ. No. WO2011/131746. In the methods, the first monospecific bivalent antibody (e.g., anti-gasdermin D antibody) and the second monospecific bivalent antibody (e.g., an antibody targeting a different antigen) are engineered to have certain substitutions at the CH3 domain that promotes heterodimer stability; the antibodies are incubated together under reducing conditions sufficient to allow the cysteines in the hinge region to undergo disulfide bond isomerization; thereby generating the bispecific antibody by Fab arm exchange. The incubation conditions may optimally be restored to non-reducing conditions. Exemplary reducing agents that may be used are 2-mercaptoethylamine (2-MEA), dithiothreitol (DTT), dithioerythritol (DTE), glutathione, tris (2-carboxyethyl)phosphine (TCEP), L-cysteine and beta-mercaptoethanol, preferably a reducing agent selected from the group consisting of: 2-mercaptoethylamine, dithiothreitol and tris (2-carboxyethyl)phosphine. For example, incubation for at least 90 min at a temperature of at least 20° C. in the presence of at least 25 mM 2-MEA or in the presence of at least 0.5 mM dithiothreitol at a pH from 5-8, for example at pH of 7.0 or at pH of 7.4 may be used.

In addition to the described gasdermin D multispecific antibodies, also provided are polynucleotide sequences capable of encoding the described gasdermin D multispecific antibodies. Vectors comprising the described polynucleotides are also provided, as are cells expressing the gasdermin D multispecific antibodies provided herein. Also described are cells capable of expressing the disclosed vectors. These cells may be mammalian cells (such as 293F cells, CHO cells), insect cells (such as Sf7 cells), yeast cells, plant cells, or bacteria cells (such as E. coli). The described antibodies may also be produced by hybridoma cells.

Gasdermin D Specific Nanobodies (VHH) Domains

In some embodiments the antigen binding protein is a VHH domain (nanobody). A unique class of ‘heavy chain only’ antibodies was discovered in 1993 in the serum of camelids. The variable domains of these antibodies, refered to herein as, VHH domains, herein also described as ‘single domain antibody’ or nanobody, consists of a single monomeric variable antibody domain. VHHs are the small naturally derived antigen-binding functional fragments (˜15 kDa) that are able to maintain the affinities and antigen-binding specificities of full length antibodies.

The VHH genes are highly homologous to the human VH3 family of clan III. Compared to the conventional human antibody VH, a few crucial amino acids are substituted in the framework 2 region (FR2) and complementarity-determining regions (CDRs) of the nanobody. The highly conserved hydrophobic amino acids (Val47, Gly49, Leu50, Trp52) in FR2 region are replaced by hydrophilic amino acids (Phe47, Glu49, Arg50, Gly52), rendering the overall structure of the nanobody more hydrophilic which contributes to high stability, solubility and resistance to aggregation. One of the unique characteristics of VHHs is their ability to target antigenic epitopes at locations which are difficult to access by large molecules such as conventional monoclonal antibodies (mAbs). In addition VHHs are also well suited in the generation of bi- and multispecific antibodies (Arbabi-Ghahroudi et al., (2017) Frontiers in Immunology Volume B: 1589). Nanobodies possess exceptional resistance to high temperatures and extreme pH. Owing to their increased hydrophilicity and single-polypeptide nature, nanobodies can be relatively efficiently produced in bacteria, yeast, mammalian cells or plant cells, enabling large-scale production at reasonable costs. The immunogenicity of VHHs domains can also be minimized by humanization.

Direct fusion to human serum albumin and PEGylation can also extend the serum half life of these molecules. As one skilled in the art would appreciate, VHH domains of the invention encompass the functional features defined for the genus of antigen binding proteins discussed herein to which the domains belong.

Gasdermin D Specific Gasdermin D Specific IgNAR Variable Domain (vNAR)

In some embodiments the antigen binding protein is an IgNAR variable domain (vNAR).

Cartilaginous fish possess a unique class of heavy-chain only antibodies termed immunoglobulin novel antigen receptors or IgNAR. IgNARs are homodimers of two heavy chains joined by disulfide bonds, and lack a light chain. Each heavy chain contains five constant domains and a variable domain (vNAR). At ˜11-15 kDa, these single domain structures are the smallest naturally occuring IgG-like proteins, and possesses a long extended CDR3. These molecules have advantages over conventional antibody molecules: they display higher thermal and chemical stability and can be engineered in their CDRs to bind to a target molecule (Cabanillas-Bernal et al., (2019) PLoS ONE 14(5): e0213394). Synthetic libraries of vNARs have been constructed that allows for the rapid generation of recombinant antibodies. As one skilled in the art would appreciate, VNAR domains of the invention encompass the functional features defined for the genus of antigen binding proteins discussed herein to which the domains belong.

Gasdermin D Specific Variable Lymphocyte Receptor (VLR) Domains

In some embodiments the antigen binding protein is a variable lymphocyte receptor (VLR) domain. It has been shown that the adaptive immune system in jawless vertebrates such as lampreys and hagfish is based on variable lymphocyte receptors (VLRs) (Sang-Chul et al., PNAS (2012) 109 (9) 3299-3304). VLRs consist of highly diverse leucine-rich repeat (LRR) modules and are characterized by an assembly of repeating 20-29 residue LRR modules. Each LRR module consists of a βstrand-α-helix structure, which forms a solenoid fold. This VLR scaffold has been developed into a binding scaffold which can be mutagenized to bind a target molecule. Variable lymphocyte receptors (VLRs) are composed of an N-terminal cap (LRRNT), the first LRR (LRR1), up to nine 24-residue variable LRR (LRRV), an end LRRV (LRRVe), a connecting peptide (CP), and the C-terminal cap (LRRCT). The sequence of the LRRV domains allows for the randomization and mutagenesis of select residues for binding to different target molecules. The modular architecture of the VLR domains allows for different combinations of LRRV domains, which further increases the potential diversity of the VLR molecules, in addition to mutagenesis, for the selection of specific binders to target molecules. Repebodies are variants of VLRs that have been redesigned at the N-terminal domain of the LRR template scaffold using an internalin-B cap structure. This modification allows for high soluble expression in bacteria. The repebody scaffold allows for variation in the numbers of LRRV modules, which in turn allows for variation in the size of the target interaction surface. LRRV modules can be added or deleted, without disruption of the overall structure of the repebody fold. As one skilled in the art would appreciate, VLR domains of the invention encompass the functional features defined for the genus of antigen binding proteins discussed herein to which the domains belong.

### Gasdermin D Specific Fibronectin Type III Domain

In some embodiments the antigen binding protein is a fibronectin type III domain (FN3). The term fibronectin type II (FN3) domain, refers to a domain occurring frequently in proteins including fibronectins, tenascin, intracellular cytoskeletal proteins, cytokine receptors and prokaryotic enzymes. The tenth type III domain of human fibronectin (10FN3) domain does not comprise any disulfide bonds, and has a monomeric structure composed of seven β-strands which resembles conventional VH domains, and contains six surface exposed loops. The BC-, DE- and FG-loops of FN3 that are structurally analogous to antibody CDRs. These surface exposed loops, or selected residues within each loop, can be randomized in order to construct libraries of fibronectin type III (FN3) domains that can be used to select novel molecules that bind to gasdermin D.

In some embodiments the FN3 domain molecule may be based on a consensus sequence of FN3 domains from human tenascin (from the tencon FN3 domain as described in U.S. Pat. No. 8,278,419, from the stabilized tencon FN3 domain as described in U.S. Pat. No. 8,569,227, or from the tencon molecule with alternative binding surfaces as described in U.S. Pat. No. 9,200,273), or from other fibronectin domains (the consensus FN3 domain as described in U.S. Pat. No. 8,293,482).

In some embodiments exemplary FN3 domains are the 15 different FN3 domains present in human tenascin C, the 15 different FN3 domains present in human fibronectin (FN), and non-natural synthetic FN3 domains as described for example in U.S. Pat. Publ. No. 2010/0216708. As one skilled in the art would appreciate, Fibronectin type III domains of the invention encompass the functional features defined for the genus of antigen binding proteins discussed herein to which the domains belong.

### Gasdermin D Specific Centyrins

In some embodiments the antigen binding protein is a centyrin. Centyrins are FN3 domain type proteins that are derived from the consensus Tenascin FN3 framework (Tencon), with specifically randomized portions of the C-strand, F-strand, CD-loop and FG-loop of the FN3 domain, to improve binding to a target molecule. Centyrins expand the FN3 library design beyond the CDR-like loops, through the identification that other regions of the FN3 domain are involved in protein protein interactions (Diem et al., (2014) Protein Engineering, Design and Selection 27(10): 419-429). As one skilled in the art would appreciate, the centyrins of the invention encompass the functional features defined for the genus of antigen binding proteins discussed herein to which the domains belong.

### Gasdermin D Specific Kringle Domains

In some embodiments the antigen binding protein is a kringle domain containing protein. Kringle domains have a rigid core structure that contains three disulfide bonds and two β-sheets, and are composed of between 78-82 amino acids. It is possible to construct a protein scaffold library based on the Kringle domain structure, wherein a region of the loop structure is mutagenized to combinations of amino acid sequences that specifically bind a target molecule, as described in U.S. Pat. Publ. No. 20120021993. As one skilled in the art would appreciate, Kringle domains of the invention encompass the functional features defined for the genus of antigen binding proteins discussed herein to which the domains belong.

### Gasdermin D Specific Designed Ankyrin Repeat Protein (DARPin)

In some embodiments the antigen binding protein is a designed ankyrin repeat protein (DARPin). DARPins are small peptides with a molecular weight of ˜17 kDa. DARPins are derived from natural Ankyrin repeat proteins (Stumpp et al. (2008) Drug Discov. Today 13 (15-16):695-701). An Ankyrin repeat module of 33 amino acid residues comprises a β-turn that is followed by two antiparallel α-helices and contains no cysteine residues. DARPins are an ensemble of repetitive structural modules that include multiple randomized Ankyrin repeat module units. DARPins form a stable protein framework that has a large potential target interaction surface. The size of the DARPin molecule can be adjusted depending upon how many Ankyrin repeat module units are incorporated into the protein. DARPins express well in bacteria, and their stability is dependent upon the number of Ankyrin repeat modules incorporated into the protein. Due to the modular assembly of DARPins, a single DARPin molecule can be directed to different epitopes on the same target molecule, or to different targets. As protein scaffolds, DARPins can be mutated in regions of the secondary structural elements that can tolerate side chain replacements, without destabilizing the structure of the molecule. Alternatively ‘loop’ DARPins incorporate continuous protracted loops that can be grafted onto the DARPin core unit, which expands the scope of potential target molecules, and retains the stability of the scaffold core (Reverdatto et al., Curr Top Med Chem (2015) 15(2)). As one skilled in the art would appreciate, DARPins of the invention encompas the functional features defined for the genus of antigen binding proteins discussed herein to which the DARPins belong.

### Gasdermin D Specific a Cystine-Knot Miniproteins

In some embodiments the antigen binding protein is a cystine-knot miniprotein.

Cystine-knot miniproteins, also known as knottins, are a class of naturally occurring cystine-rich peptides. Knottins, are a structural family (typically 25-50 amino acids in length) characterized by a core of antiparallel β-strands stabilized by at least three disulfide bonds. In a characteristic cystine-knot motif, the first and fourth and the second and fifth cysteine residues form disulfide bonds. A disulfide bond formed between the third and sixth cysteine residues passes through these first two disulfides, creating a macrocyclic knot. Knottins possess loop regions of variable length that are able to tolerate amino acid mutations and can be engineered to bind to a target molecule. The engineered loop may include amino acid substitutions, insertions, and/or deletions in an existing loop of the knottin peptide. The knottin peptide scaffold is able to tolerate replacement of the natural loop, with an engineered loop that has the desired target molecule binding property. The knottin structural scaffold possesses excellent structural and thermal stability. The scaffold for the knottin peptide, may be a peptide described in the online KNOTTIN database. As one skilled in the art would appreciate, cysteine-knot miniproteins of the invention encompass the functional features defined for the genus of antigen binding proteins discussed herein to which the miniproteins belong.

### Gasdermin D Specific Sso7d Proteins

In some embodiments the antigen binding protein is a Sso7d derived protein.

Sso7d is a protein from the hyperthermophilic archaeon Sulfolobus solfataricus that lacks cysteine residues and is thermally stable. It is a small molecule of ˜7 kDa. Sso7d and is a DNA binding protein. Its DNA-binding surface can be mutagenised in order to be engineered to bind other target molecules. The Sso7d scaffold displays high, thermal, chemical and pH stability, and can be easily produced by recombinant expression (Gera et al., (2011) J Mol Biol 409(4): 601-616). As one skilled in the art would appreciate, Sso7d proteins of the invention encompass the functional features defined for the genus of antigen binding proteins discussed herein to which the miniproteins belong.

### Gasdermin D Specific Affibody

In some embodiments the antigen binding protein is an affibody.

Affibody molecules utilise a scaffold protein based on the structure of the immunoglobulin G binding domain (Z domain) of protein A from Staphylococus aureus (Nygren (2008) FEBS J. 275 (11):2668-2676). They are therefore based on a small three-helix bundle domain framework that is ˜6 kDa in size. There are numerous advantages to the use of this protein scaffold. Affibodies are amenable to production in a variety of host cells, or, due to their small size, can be chemically synthesized. They fold rapidly, are resistant to proteolysis and do not contain any cysteine residues. The three helix bundles can be polymerized, with multiple domains, which can increase the avidity of the molecule, or allow for the specificity to multiple different epitopes or target molecules in one protein. The scaffold allows for mutagenesis on a secondary structural element of the protein; there are more than 13 solvent accessible residues on the surface of helices 1 and 2 of the three helix bundle, and mutagenesis does not destabilize the protein. This allows for the generation of novel combinatorial libraries of affibodies in order to screen for selectivity against a target molecule (Reverdatto et al., Curr Top Med Chem (2015) 15(2)). As one skilled in the art would appreciate, affibodies of the invention encompass the functional features defined for the genus of antigen binding proteins discussed herein to which the affibodies belong.

### Gasdermin Specific Affimers

In some embodiments the antigen binding protein is an affimer. Affimer is a registered trade mark, and the scaffold/polypeptides of Affimer technology is based on the cystatin family (Tiede et al., eLife (2017) 6 e24903). The Adhiron scaffold is a synthetic protein originally based on the consensus sequence of the cystatin family, and is closely related structurally to an engineered scaffold based on human stefin A. The cystatins share a common tertiary structure of an alpha-helix lying on top of an anti-parallel β-sheet. The two loops connecting the four antiparallel β-sheet, together with the N-terminal sequence can be mutagenized to bind a target molecule. As one skilled in the art would appreciate, affimers of the invention encompass the functional features of the antigen binding proteins discussed herein.

### Gasdermin D Specific Anticalins

In some embodiments the antigen binding protein is an anticalin.

Anticalins are proteins that are derived from the lipocalin family (Skerra (2008) FEBS J. 275 (11):2677-2683). Lipocalins are a family of small (˜20 kDa, 150-160 residues), often monomeric extracellular proteins, that are found widely in prokaryotes and eukaryotes. The central element in the folding architecture of the lipocalins is a cylindrical 3-pleated sheet structure, the so-called β-barrel, which is made up of eight nearly circularly arranged antiparallel β-strands. The β-barrel is closed on one end by dense amino acid packing as well as by loop segments, forming a cup-shaped β-barrel core connected with four structurally variable loops. The binding pocket of the lipocalin occurs in four loop peptides together with the adjacent residues in the β-barrel core. Lipocalins have a high degree of structural similarity in the β-barrel, and display broad structural diversity in the four-loop region. This four loop region resembles the organization of antibody antigen binding sites, however, unlike the usually flat interface of antibody CDRs, the lipocalins possess a deep ligand pocket. This four loop region can be mutagenized to bind to specific target molecules, as described in U.S Pat. Publ. No. 20160280747. It is possible to generate at least 16-24 randomized residues per loop in order to design anticalin combinatorial libraries for selection. Anticalins are structurally very stable, with melting temperatures exceeding 70° C., they are not affected by permutations in the loop region. They are not glycosylated which makes them suitable for expression in yeast or bacteria. As one skilled in the art would appreciate, anticalins of the invention encompass the functional features defined for the genus of antigen binding proteins discussed herein to which the anticalins belong.

### Gasdermin D Specific Affilins

In some embodiments the antigen binding protein is an affilin.

The term Affilin® (registered trademark of Navigo Proteins GmbH, formerly known as Scil Proteins GmbH) as used herein refers to non-immunoglobulin derived binding proteins based on mutagenized versions of ubiquitin or γ-B crystallin muteins (Ebersbach et al. (2007) J. Mol. Biol. 372 (1): 172-185). γ-B crystallin has two identical domains that is predominantly β-sheet in structure. In comparison ubiquitin consists of three and a half α-turns, and a five stranded β-sheet. In γ-B crystalline the β-sheet provides the potential binding surface for target molecules, and eight surface residues can be mutagenized. The ubiquitin scaffold can utilize six residues in the β-sheet region for target binding, and more extensive randomization can also be adopted in the α-turns. In addition the ubiquitin molecules can also be dimerized in a head to tail orientation. Affibodies are characterized by their stability to changes in pH, thermal denaturation and high concentrations of denaturing agents (Reverdatto et al., Curr Top Med Chem (2015) 15(2)). As one skilled in the art would appreciate, affilins of the invention encompass the functional features defined for the genus of antigen binding proteins discussed herein to which the affilins belong.

### Gasdermin D Specific Affitins

In some embodiments the antigen binding protein is an affitin.

Affitins are proteins that are structurally derived from the DNA binding protein Sac7d, found in Sulfolobus acidocaldarius (Krehenbrink et al. (2008) J. Mol. Biol. 383 (5): 1058-1068). Mutagenizing amino acids of the binding surface of Sac7d allows for the affitin to selectively bind a target molecule. As one skilled in the art would appreciate, affitins of the invention encompass the functional features defined for the genus of antigen binding proteins discussed herein to which the affitins belong.

### Gasdermin D Specific Fynomers

In some embodiments the antigen binding protein is a fynomer.

Fynomers are proteins that are structurally derived from the Src tyrosine kinase Fyn SH3 domain (Grabulovski et al. (2007) J. Biol. Chem. 282 (5):3196-3204). The FynSH3 domain binds to proline rich peptides containing a PXXP binding motif, and its sequence is fully conserved between human, mouse, rat and gibbons. Fyn SH3 is composed of two antiparallel β-sheets and contains two flexible loops that are positioned to interact with other proteins. These loops can be mutagenized to selectively bind a target molecule. Up to six residues in each loop can be varied without significantly reducing the solubility and folding properties of the protein. Fynomers are attractive scaffolds as antigen binding proteins because they can be solubly expressed in bacteria in high amounts, are monomeric and do not aggregate when stored in solution, lack cysteine residues, are thermally stable (with melting temperatures greater than 70° C.) and are of human origin so are non-immunogenic (Reverdatto et al., Curr Top Med Chem (2015) 15(2)). As one skilled in the art would appreciate, fynomers of the invention encompass the functional features defined for the genus of antigen binding proteins discussed herein to which the fynomers belong.

### Gasdermin D Specific Fc Fusion Proteins

In some embodiments the antigen binding protein is an Fc fusion protein. Fc-based fusion proteins are composed of an immunoglobulin Fc domain that is directly linked to another peptide. The fused partner can be any other protein molecule of interest. Attachment of the fusion protein to the Fc-domain provides a number of additional beneficial biological and pharmacological properties. The presence of the Fc domain increases the plasma half-life of the molecule, which can prolong the therapeutic activity of the molecule. In addition the Fc domain folds independently of the fusion partner and can improve the solubility and stability of the partner molecule. Fc fusions can be modified to polymerize into well-defined complexes containing twelve fused partners, therefore increasing the avidity, and potency of the molecule. In some embodiments the antigen binding protein is an Fc fusion protein, wherein the fusion partner binds to gasdermin D. In some embodiments the antigen binding protein is an Fc fusion protein, wherein the fusion partner binds to gasdermin D and wherein the fusion protein is the N-terminal domain of gasdermin D. In some embodiments the antigen binding domain is an Fc fusion protein, wherein the fusion partner binds to gasdermin D and the Fc region blocks the multimeric gasdermin D pore. One skilled in the art would appreciate that the Fc fusion proteins can be made, where the fusion partner is any antigen binding protein discussed above.

### Gasdermin D Specific Aptamers

In some embodiments, the inhibitor of the invention is an aptamer. Aptamers are either non-proteinaceous, for example nucleic acids or proteinaceous, for example peptides that bind to a target molecule.

The invention provides an aptamer that binds to gasdermin D and inhibits gasdermin D, such as wherein the aptamer neutralizes gasdermin D.

The invention provides an aptamer that binds to gasdermin D and neutralizes gasdermin D.

In some embodiments, the aptamer binds to gasdermin D and inhibits gasdermin D, wherein the inhibition of gasdermin D is the inhibition of an activity of gasdermin D, such as wherein the aptamer neutralizes an activity of gasdermin D and/or the inhibition of gasdermin D is the inhibition of a function of gasdermin D, such as wherein the aptamer neutralizes a function of gasdermin D.

In some embodiments, the aptamer binds to gasdermin D and neutralizes gasdermin D, wherein the neutralization of gasdermin D is the neutralization of an activity of gasdermin D, and/or the neutralization of gasdermin D is the neutralization of a function of gasdermin D.

In some embodiments the aptamer is an extracellular inhibitor that binds to gasdermin D.

In some embodiments aptamer binds to gasdermin D on the cell surface.

In some embodiments, the aptamer binds to gasdermin D and does not cross the cell membrane, unless it is bound to gasdermin D.

In some embodiments, the aptamer is a large molecule.

In some embodiments, the aptamer has a molecular weight of >2 kDa, >3 kDa, >4 kDa, >5 kDa, >6 kDa, >7 kDa, >8 kDa, >9 kDa or >10 kDa.

In some embodiments, the aptamer binds to the N-terminal domain of gasdermin D.

In some embodiments the aptamer of the invention binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer of the invention binds to an epitope comprising SEQ ID NO: 9. In some embodiments the aptamer of the invention binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer of the invention binds to SEQ ID NO: 9.

In some embodiments the aptamer binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an isolated peptide of SEQ ID NO: 9.

In some embodiments, the aptamer binds to gasdermin D and inhibits its association with lipids, such as wherein the aptamer binds to gasdermin D and neutralizes the association of gasdermin D with lipids.

In some embodiments, the aptamer binds to gasdermin D and inhibits the association of gasdermin D with phosphatidylinositol 4-phosphate and/or phosphatidylinositol 4,5-bisphosphate, such as wherein the aptamer binds to gasdermin D and neutralizes the association of gasdermin D with phosphatidylinositol 4-phosphate and/or phosphatidylinositol 4,5-bisphosphate.

In some embodiments, the aptamer binds to gasdermin D and inhibits oligomerisation of gasdermin D, such as wherein the aptamer neutralizes oligomerisation of gasdermin D.

In some embodiments, the aptamer inhibits protein-protein interactions between the gasdermin D subunits, such as wherein the aptamer neutralizes protein-protein interactions between gasdermin D subunits.

In some embodiments, the aptamer binds to a gasdermin D multimeric pore.

In some embodiments, the aptamer binds to a gasdermin D multimeric pore and blocks the pore. In some embodiments, the aptamer binds to a gasdermin D multimeric pore and disrupts protein-protein interactions between gasdermin D subunits of the pore.

In some embodiments, the aptamer binds to a gasdermin D subunit of the multimeric pore. In some embodiments, the aptamer binds to a gasdermin D subunit of the multimeric pore and blocks the pore.

In some embodiments, the aptamer binds to a gasdermin D subunit of the multimeric pore, and disrupts the protein-protein interactions between the subunits of the pore.

In some embodiments the aptamer can inhibit cell death induced by pyroptosis, such as wherein the aptamer neutralizes cell death induced by pyroptosis.

In some embodiments the antibody or antigen binding fragment thereof inhibits release of IL-1β and/or IL-18, such as wherein the aptamer neutralizes release of IL-1β and/or IL-18.

In some embodiments, the aptamer can inhibit cell death induced by pyroptosis by at least 5%, at least 10%, at least 20%, at least 30%, at least 40%, at least 50%, at least 60%, at least 70%, at least 80%, at least 90%, or about 100%, such as 100% for example in a THP-1 cell assay as described herein.

In some embodiments, the aptamer inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl (for example 50,000 cell or 65,000 cells in 100 μl), wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the aptamer can delay cell death induced by pyroptosis by >0.1 hours, >0.2 hours, >0.5 hours, >0.75 hours, >1 hour, >2 hours, >3 hours, >4 hours, or >6 hours for example in a THP-1 cell assay as described herein.

In some embodiments, the aptamer delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl (for example 50,000 cell or 65,000 cells in 100 μl), wherein the THP-1 cells have been pre-treated with phorbol 12-myristate 13-acetate for 16 hours, and wherein the THP-1 cells have been induced with 20 μM nigericin.

In some embodiments, the aptamer cross reacts with an old world monkey gasdermin D or a new world monkey gasdermin D. The advantages of such cross-reactivity are discussed above herein.

In some embodiments, the aptamer is non-proteinaceous.

In some embodiments the aptamer is an oligonucleotide, a peptide, or a SOMamer.

Gasdermin D Specific Aptamers that Bind to an Epitope of Gasdermin D and Inhibit Association with Lipids

In some embodiments, the aptamer binds to the N-terminal domain of gasdermin D, wherein the aptamer binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an epitope comprising SEQ ID NO: 9 and wherein the aptamer binds to gasdermin D and inhibits its association with lipids.

In some embodiments, the aptamer binds to the N-terminal domain of gasdermin D, wherein the aptamer binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an epitope comprising SEQ ID NO: 9 and wherein the aptamer binds to gasdermin D and inhibits its association with phosphatidylinositol 4-phosphate and/or phosphatidylinositol 4,5-bisphosphate.

In some embodiments, the aptamer binds to the N-terminal domain of gasdermin D, wherein the aptamer binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an epitope comprising SEQ ID NO: 9, wherein the aptamer binds to gasdermin D and inhibits its association with lipids, and wherein the aptamer can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the aptamer inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the aptamer binds to the N-terminal domain of gasdermin D, wherein the aptamer binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an epitope comprising SEQ ID NO: 9, wherein the aptamer binds to gasdermin D and inhibits its association with phosphatidylinositol 4-phosphate and/or phosphatidylinositol 4,5-bisphosphate, and wherein the aptamer can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the aptamer inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the aptamer binds to the N-terminal domain of gasdermin D, wherein the aptamer binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an epitope comprising SEQ ID NO: 9, wherein the aptamer binds to gasdermin D and inhibits its association with lipids, and wherein the aptamer can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the aptamer delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

In some embodiments, the aptamer binds to the N-terminal domain of gasdermin D, wherein the aptamer binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an epitope comprising SEQ ID NO: 9, wherein the aptamer binds to gasdermin D and inhibits its association with phosphatidylinositol 4-phosphate and/or phosphatidylinositol 4,5-bisphosphate, and wherein the aptamer can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the aptamer delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

Gasdermin D Specific Aptamers that Bind to a Sequence of Gasdermin D and Inhibit Association with Lipids

In some embodiments, the aptamer binds to the N-terminal domain of gasdermin D, wherein the aptamer binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to SEQ ID NO: 9, and wherein the aptamer binds to gasdermin D and inhibits its association with lipids.

In some embodiments, the aptamer binds to the N-terminal domain of gasdermin D, wherein the aptamer binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to SEQ ID NO: 9, and wherein the aptamer binds to gasdermin D and inhibits its association with phosphatidylinositol 4-phosphate and/or phosphatidylinositol 4,5-bisphosphate.

In some embodiments, the aptamer binds to the N-terminal domain of gasdermin D, wherein the aptamer binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to SEQ ID NO: 9, wherein the aptamer binds to gasdermin D and inhibits its association with lipids, and wherein the aptamer can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the aptamer inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the aptamer binds to the N-terminal domain of gasdermin D, wherein the aptamer binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to SEQ ID NO: 9, wherein the aptamer binds to gasdermin D and inhibits its association with phosphatidylinositol 4-phosphate and/or phosphatidylinositol 4,5-bisphosphate, and wherein the aptamer can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the aptamer inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the aptamer binds to the N-terminal domain of gasdermin D, wherein the aptamer binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to SEQ ID NO: 9, wherein the aptamer binds to gasdermin D and inhibits its association with lipids, and wherein the aptamer can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the aptamer delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

In some embodiments, the aptamer binds to the N-terminal domain of gasdermin D, wherein the aptamer binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to SEQ ID NO: 9, wherein the aptamer binds to gasdermin D and inhibits its association with phosphatidylinositol 4-phosphate and/or phosphatidylinositol 4,5-bisphosphate, and wherein the aptamer can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the aptamer delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

Gasdermin D Specific Aptamers that Bind to an Isolated Peptide of Gasdermin D and Inhibit Association with Lipids

In some embodiments, the aptamer binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to SEQ ID NO: 9, and wherein the aptamer binds to gasdermin D and inhibits its association with lipids.

In some embodiments, the aptamer binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an isolated peptide of SEQ ID NO: 9, and wherein the aptamer binds to gasdermin D and inhibits its association with phosphatidylinositol 4-phosphate and/or phosphatidylinositol 4,5-bisphosphate.

In some embodiments, the aptamer binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an isolated peptide of SEQ ID NO: 9, wherein the aptamer binds to gasdermin D and inhibits its association with lipids, and wherein the aptamer can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the aptamer inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the aptamer binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an isolated peptide of SEQ ID NO: 9, wherein the aptamer binds to gasdermin D and inhibits its association with phosphatidylinositol 4-phosphate and/or phosphatidylinositol 4,5-bisphosphate, and wherein the aptamer can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the aptamer inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the aptamer binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an isolated peptide of SEQ ID NO: 9, wherein the aptamer binds to gasdermin D and inhibits its association with lipids, and wherein the aptamer can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the aptamer delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

In some embodiments, the aptamer binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an isolated peptide of SEQ ID NO: 9, wherein the aptamer binds to gasdermin D and inhibits its association with phosphatidylinositol 4-phosphate and/or phosphatidylinositol 4,5-bisphosphate, and wherein the aptamer can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the aptamer delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

Gasdermin D Specific Aptamers that Bind to an Epitope of Gasdermin D and Inhibit Oligomerisation

In some embodiments, the aptamer binds to the N-terminal domain of gasdermin D, wherein the aptamer binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an epitope comprising SEQ ID NO: 9, and wherein the aptamer inhibits oligomerisation of gasdermin D.

In some embodiments, the aptamer binds to the N-terminal domain of gasdermin D, wherein the aptamer binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an epitope comprising SEQ ID NO: 9, and wherein the aptamer inhibits protein-protein interactions between the gasdermin D subunits, thereby preventing oligomerisation to form a multimeric pore.

In some embodiments, the aptamer binds to the N-terminal domain of gasdermin D, wherein the aptamer binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an epitope comprising SEQ ID NO: 9, wherein the aptamer inhibits oligomerisation of gasdermin D, and wherein the aptamer can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the aptamer inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the aptamer binds to the N-terminal domain of gasdermin D, wherein the aptamer binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an epitope comprising SEQ ID NO: 9, wherein the aptamer inhibits protein-protein interactions between the gasdermin D subunits, thereby preventing oligomerisation to form a multimeric pore, and wherein the aptamer can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the aptamer inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the aptamer binds to the N-terminal domain of gasdermin D, wherein the aptamer binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an epitope comprising SEQ ID NO: 9, wherein the aptamer inhibits oligomerisation of gasdermin D and prevents formation of the pore, and wherein the aptamer can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the aptamer delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

In some embodiments, the aptamer binds to the N-terminal domain of gasdermin D, wherein the aptamer binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an epitope comprising SEQ ID NO: 9, wherein the aptamer inhibits protein-protein interactions between the gasdermin D subunits, thereby preventing oligomerisation to form a multimeric pore, and wherein the aptamer can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the aptamer delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

Gasdermin D Specific Aptamers that Bind to a Sequence of Gasdermin D and Inhibit Oligomerisation

In some embodiments, the aptamer binds to the N-terminal domain of gasdermin D, wherein the aptamer binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to SEQ ID NO: 9, and wherein the aptamer inhibits oligomerisation of gasdermin D.

In some embodiments, the aptamer binds to the N-terminal domain of gasdermin D, wherein the aptamer binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to SEQ ID NO: 9, and wherein the aptamer inhibits protein-protein interactions between the gasdermin D subunits, thereby preventing oligomerisation to form a multimeric pore.

In some embodiments, the aptamer binds to the N-terminal domain of gasdermin D, wherein the aptamer binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to SEQ ID NO: 9, wherein the aptamer inhibits oligomerisation of gasdermin D, and wherein the aptamer can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the aptamer inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the aptamer binds to the N-terminal domain of gasdermin D, wherein the aptamer binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to SEQ ID NO: 9, wherein the aptamer inhibits protein-protein interactions between the gasdermin D subunits, thereby preventing oligomerisation to form a multimeric pore, and wherein the aptamer can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the aptamer inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the aptamer binds to the N-terminal domain of gasdermin D, wherein the aptamer binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to SEQ ID NO: 9, wherein the aptamer inhibits oligomerisation of gasdermin D, and wherein the aptamer can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the aptamer delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

In some embodiments, the aptamer binds to the N-terminal domain of gasdermin D, wherein the aptamer binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to SEQ ID NO: 9, wherein the aptamer inhibits protein-protein interactions between the gasdermin D subunits, thereby preventing oligomerisation to form a multimeric pore, and wherein the aptamer can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the aptamer delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

Gasdermin D Specific Aptamers that Bind to an Isolated Peptide of Gasdermin D and Inhibit Oligomerisation

In some embodiments, the aptamer binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an isolated peptide of SEQ ID NO: 9, and wherein the aptamer inhibits oligomerisation of gasdermin D.

In some embodiments, the aptamer binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an isolated peptide of SEQ ID NO: 9, and wherein the aptamer inhibits protein-protein interactions between the gasdermin D subunits, thereby preventing oligomerisation to form a multimeric pore.

In some embodiments, the aptamer binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an isolated peptide of SEQ ID NO: 9, wherein the aptamer inhibits oligomerisation of gasdermin D, and wherein the aptamer can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the aptamer inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the aptamer binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an isolated peptide of SEQ ID NO: 9, wherein the aptamer inhibits protein-protein interactions between the gasdermin D subunits, thereby preventing oligomerisation to form a multimeric pore, and wherein the aptamer can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the aptamer inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the aptamer binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an isolated peptide of SEQ ID NO: 9, wherein the aptamer inhibits oligomerisation of gasdermin D, and wherein the aptamer can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the aptamer delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

In some embodiments, the aptamer binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an isolated peptide of SEQ ID NO: 9, wherein the aptamer inhibits protein-protein interactions between the gasdermin D subunits, thereby preventing oligomerisation to form a multimeric pore, and wherein the aptamer can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the aptamer delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

Gasdermin D Specific Aptamers that Bind to an Epitope of Gasdermin D and Inhibit a Gasdermin D Multimeric Pore

In some embodiments, the aptamer binds to the N-terminal domain of gasdermin D, wherein the aptamer binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an epitope comprising SEQ ID NO: 9, and wherein the aptamer binds to a gasdermin D multimeric pore and blocks the pore.

In some embodiments, the aptamer binds to the N-terminal domain of gasdermin D, wherein the aptamer binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an epitope comprising SEQ ID NO: 9, wherein the aptamer binds to a gasdermin D multimeric pore and blocks the pore and wherein the aptamer can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the aptamer inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the aptamer binds to the N-terminal domain of gasdermin D, wherein the aptamer binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an epitope comprising SEQ ID NO: 9, wherein the aptamer binds to a gasdermin D multimeric pore and blocks the pore, and wherein the aptamer can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the aptamer delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

In some embodiments, the aptamer binds to the N-terminal domain of gasdermin D, wherein the aptamer binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an epitope comprising SEQ ID NO: 9, wherein the aptamer binds to a gasdermin D multimeric pore, and disrupts the protein-protein interactions between the subunits of the pore.

In some embodiments, the aptamer binds to the N-terminal domain of gasdermin D, wherein the aptamer binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an epitope comprising SEQ ID NO: 9, wherein the aptamer binds to a gasdermin D multimeric pore, and disrupts the protein-protein interactions between the subunits of the pore and wherein the aptamer can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the aptamer inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the aptamer binds to the N-terminal domain of gasdermin D, wherein the aptamer binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an epitope comprising SEQ ID NO: 9, wherein the aptamer binds to a gasdermin D multimeric pore, and disrupts the protein-protein interactions between the subunits of the pore, and wherein the aptamer can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the aptamer delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

Gasdermin D Specific Aptamers that Bind to an Epitope of a Gasdermin D Subunit of the Multimeric Pore, and Inhibit a Gasdermin D Multimeric Pore

In some embodiments, the aptamer binds to the N-terminal domain of gasdermin D, wherein the aptamer binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an epitope comprising SEQ ID NO: 9, and wherein the aptamer binds to a gasdermin D subunit of the multimeric pore and blocks the pore.

In some embodiments, the aptamer binds to the N-terminal domain of gasdermin D, wherein the aptamer binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an epitope comprising SEQ ID NO: 9, wherein the aptamer binds to a gasdermin D subunit of the multimeric pore and blocks the pore, and wherein the aptamer can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the aptamer inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the aptamer binds to the N-terminal domain of gasdermin D, wherein the aptamer binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an epitope comprising SEQ ID NO: 9, wherein the aptamer binds to a gasdermin D subunit of the multimeric pore and blocks the pore, and wherein the aptamer can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the aptamer delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

In some embodiments, the aptamer binds to the N-terminal domain of gasdermin D, wherein the aptamer binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an epitope comprising SEQ ID NO: 9, and wherein the aptamer binds to a gasdermin D subunit of the multimeric pore, and disrupts the protein-protein interactions between the subunits of the pore.

In some embodiments, the aptamer binds to the N-terminal domain of gasdermin D, wherein the aptamer binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an epitope comprising SEQ ID NO: 9, wherein the aptamer binds to a gasdermin D subunit of the multimeric pore, and disrupts the protein-protein interactions between the subunits of the pore and wherein the aptamer can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, optionally wherein the aptamer inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the aptamer binds to the N-terminal domain of gasdermin D, wherein the aptamer binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an epitope comprising SEQ ID NO: 9, wherein the aptamer binds to a gasdermin D subunit of the multimeric pore, and disrupts the protein-protein interactions between the subunits of the pore, and wherein the aptamer can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the aptamer delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

Gasdermin D Specific Aptamers that Bind to a Sequence of Gasdermin D and Inhibit a Gasdermin D Multimeric Pore

In some embodiments, the aptamer binds to the N-terminal domain of gasdermin D, wherein the aptamer binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to SEQ ID NO: 9, and wherein the aptamer binds to a gasdermin D multimeric pore and blocks the pore.

In some embodiments, the aptamer binds to the N-terminal domain of gasdermin D, wherein the aptamer binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to SEQ ID NO: 9, wherein the aptamer binds to a gasdermin D multimeric pore and blocks the pore and wherein the aptamer can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the aptamer inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the aptamer binds to the N-terminal domain of gasdermin D, wherein the aptamer binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to SEQ ID NO: 9, wherein the aptamer binds to a gasdermin D multimeric pore and blocks the pore, and wherein the aptamer can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the aptamer delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

In some embodiments, the aptamer binds to the N-terminal domain of gasdermin D, wherein the aptamer binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to SEQ ID NO: 9, and wherein the aptamer binds to a gasdermin D multimeric pore, and disrupts the protein-protein interactions between the subunits of the pore.

In some embodiments, the aptamer binds to the N-terminal domain of gasdermin D, wherein the aptamer binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to SEQ ID NO: 9, wherein the aptamer binds to a gasdermin D multimeric pore, and disrupts the protein-protein interactions between the subunits of the pore and wherein the aptamer can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the aptamer inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the aptamer binds to the N-terminal domain of gasdermin D, wherein the aptamer binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to SEQ ID NO: 9, wherein the aptamer binds to a gasdermin D multimeric pore, and disrupts the protein-protein interactions between the subunits of the pore, and wherein the aptamer can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the aptamer delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

Gasdermin D Specific Aptamers that Bind to a Sequence of a Gasdermin D Subunit of the Multimeric Pore, and Inhibit a Gasdermin D Multimeric Pore

In some embodiments, the aptamer binds to the N-terminal domain of gasdermin D, wherein the aptamer binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to SEQ ID NO: 9, and wherein the aptamer binds to a gasdermin D subunit of the multimeric pore and blocks the pore.

In some embodiments, the aptamer binds to the N-terminal domain of gasdermin D, wherein the aptamer binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to SEQ ID NO: 9, wherein the aptamer binds to a gasdermin D subunit of the multimeric pore and blocks the pore and wherein the aptamer can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the aptamer inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the aptamer binds to the N-terminal domain of gasdermin D, wherein the aptamer binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to SEQ ID NO: 9, wherein the aptamer binds to a gasdermin D subunit of the multimeric pore and blocks the pore, and wherein the aptamer can delay cell death induced by pyroptosis by >0.1 hours, >0.2 hours, >0.5 hours, >0.75 hours, >1 hour, >2 hours, >3 hours, >4 hours, or >6 hours, optionally wherein the aptamer delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, for example in a THP-1 cell assay as described herein, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

In some embodiments, the aptamer binds to the N-terminal domain of gasdermin D, wherein the aptamer binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to SEQ ID NO: 9, and wherein the aptamer binds to a gasdermin D subunit of the multimeric pore, and disrupts the protein-protein interactions between the subunits of the pore.

In some embodiments, the aptamer binds to the N-terminal domain of gasdermin D, wherein the aptamer binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to SEQ ID NO: 9, wherein the aptamer binds to a gasdermin D subunit of the multimeric pore, and disrupts the protein-protein interactions between the subunits of the pore and wherein the aptamer can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the aptamer inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the aptamer binds to the N-terminal domain of gasdermin D, wherein the aptamer binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to SEQ ID NO: 9, wherein the aptamer binds to a gasdermin D subunit of the multimeric pore, and disrupts the protein-protein interactions between the subunits of the pore, and wherein the aptamer can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the aptamer delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

Gasdermin D Specific Aptamers that Bind to an Isolated Peptide of Gasdermin D and Inhibit a Gasdermin D Multimeric Pore

In some embodiments, the aptamer binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an isolated peptide of SEQ ID NO: 9, and wherein the aptamer binds to a gasdermin D multimeric pore and blocks the pore.

In some embodiments, the aptamer binds to the N-terminal domain of gasdermin D and binds to an isolated peptide of o SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an isolated peptide of SEQ ID NO: 9, wherein the aptamer binds to a gasdermin D multimeric pore and blocks the pore and wherein the aptamer can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the aptamer inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the aptamer binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an isolated peptide of SEQ ID NO: 9, wherein the aptamer binds to a gasdermin D multimeric pore and blocks the pore, and wherein the aptamer can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the aptamer delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

In some embodiments, the aptamer binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an isolated peptide of SEQ ID NO: 9, and wherein the aptamer binds to a gasdermin D multimeric pore, and disrupts the protein-protein interactions between the subunits of the pore.

In some embodiments, the aptamer binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an isolated peptide of SEQ ID NO: 9, wherein the aptamer binds to a gasdermin D multimeric pore, and disrupts the protein-protein interactions between the subunits of the pore and wherein the aptamer can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the aptamer inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the aptamer binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an isolated peptide of SEQ ID NO: 9, wherein the aptamer binds to a gasdermin D multimeric pore, and disrupts the protein-protein interactions between the subunits of the pore, and wherein the aptamer can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the aptamer delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

Gasdermin D Specific Aptamers that Bind to an Isolated Peptide of a Gasdermin D Subunit of the Multimeric Pore, and Inhibit a Gasdermin D Multimeric Pore

In some embodiments, the aptamer binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an isolated peptide of SEQ ID NO: 9, and wherein the aptamer binds to a gasdermin D subunit of the multimeric pore and blocks the pore.

In some embodiments, the aptamer binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an isolated peptide of SEQ ID NO: 9, wherein the aptamer binds to a gasdermin D subunit of the multimeric pore and blocks the pore and wherein the aptamer can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the aptamer inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the aptamer binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an isolated peptide of SEQ ID NO: 9, wherein the aptamer binds to a gasdermin D subunit of the multimeric pore and blocks the pore, and wherein the aptamer can delay cell death induced by pyroptosis by >0.1 hours, >0.2 hours, >0.5 hours, >0.75 hours, >1 hour, >2 hours, >3 hours, >4 hours, or >6 hours, optionally wherein the aptamer delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, for example in a THP-1 cell assay as described herein, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

In some embodiments, the aptamer binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an isolated peptide of SEQ ID NO: 9, and wherein the aptamer binds to a gasdermin D subunit of the multimeric pore, and disrupts the protein-protein interactions between the subunits of the pore.

In some embodiments, the aptamer binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an isolated peptide of SEQ ID NO: 9, wherein the aptamer binds to a gasdermin D subunit of the multimeric pore, and disrupts the protein-protein interactions between the subunits of the pore and wherein the aptamer can inhibit cell death induced by pyroptosis by at least 5%, at least 10%, at least 20%, at least 30%, at least 40%, at least 50%, at least 60%, at least 70%, at least 80%, at least 90%, or about 100%, such as 100%, for example in a THP-1 cell assay as described herein, optionally wherein the aptamer inhibits cell death induced by pyroptosis when administered to human THP-1 cells by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000-65,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.

In some embodiments, the aptamer binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the aptamer binds to an isolated peptide of SEQ ID NO: 9, wherein the aptamer binds to a gasdermin D subunit of the multimeric pore, and disrupts the protein-protein interactions between the subunits of the pore, and wherein the aptamer can delay cell death induced by pyroptosis by >0.1 hours, >0.2 hours, >0.5 hours, >0.75 hours, >1 hour, >2 hours, >3 hours, >4 hours, or >6 hours, for example in a THP-1 cell assay as described herein, optionally wherein the aptamer delays cell death induced by pyroptosis, when administered to human THP-1 cells, by >0.1 hours, >0.2 hours, >0.5 hours, >0.75 hours, >1 hour, >2 hours, >3 hours, >4 hours, or >6 hours, wherein the THP-1 cells are at a cell density of 50,000-65,000 cells in 100 μl, wherein the THP-1 cells have been pre-treated with phorbol 12-myristate 13-acetate for 16 hours, and wherein the THP-1 cells have been induced with 20 μM nigericin.

### Gasdermin D Specific Oligonucleotide Aptamers

In some embodiments the aptamer is non-proteinaceous. In some embodiments, the aptamer is an oligonucleotide aptamer. Oligonucleotide aptamers are short single-stranded oligonucleotides, either single stranded DNA (ssDNA) or RNA, with a specific and complex three-dimensional shape, that bind to target molecules. The molecular recognition of aptamers is based on structure compatibility and intermolecular interactions, including electrostatic forces, van der Waals interactions, hydrogen bonding, and π-π stacking interactions of aromatic rings with the target molecule. Oligonucleotide based aptamers have high affinity and specificity for their target ligand. Oligonucleotide aptamers bind precisely and strongly because they extend the surface contact with their cognate targets through adaptive conformational changes, resulting in the creation of specific binding sites. Nucleic acid aptamers, in addition, are non-toxic and lack immunogenicity.

Aptamers are usually designed in vitro from large libraries of random nucleic acids by Systematic Evolution of Ligands by Exponential Enrichment (SELEX). Oligonucleotide aptamers are nucleic acids, (RNA, DNA or XNA) molecules that are capable of folding into 3-D structures to selectively bind to a molecular target. SELEX (Systematic evolution of ligands by exponential enrichment) is a technology that allows for the rapid interrogation of large synthetic oligonucleotide libraries to bind to their molecular target (Dunn et al., (2017) Nature Reviews Chemistry 1:0076). As the person skilled in the art would appreciate, a typical SELEX experiment involves selecting ˜10¹⁴-10¹⁶ random sequences, with a length of 20 to 100 base pairs and comprises: 1) screening the condition of incubation with the target molecule 2) selecting molecules that bind to the target molecule; 3) eluting the bound species, and 4) amplification of the nucleic acids. This process is iteratively repeated to select for sequences that bind more tightly to the target molecule. By modulating various parameters of the selection experiment, including the nature of the target molecule, the length of the randomized region of the original oligonucleotide library, and the selection stringency, a broad array of multifunctional aptamers can be obtained. In some embodiments the target molecule used in the selection process of the oligonucleotide aptamer is SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the target molecule is SEQ ID NO: 9.

Traditional aptamers using DNA or RNA libraries have limited chemical diversity, and lack, or have weak, hydrophobic interactions with their cognate target. In some embodiments the aptamer comprises modifications of the sugar unit, the nucleobase, or the backbone of the nucleotide. An aptamer can be partially or completely substituted with one or more modifications, and conjugated with functional molecules (Shuaijian et al., (2017) Int J Mol Sci. 18(8): 1683).

Non-limiting examples of sugar modifications include modifications at the 2′ position of the (deoxy)-ribose sugar unit such as: 2′-amino pyrimidines, 2′-fluoro pyrimidines, 2′-O-methyl ribose purines and replacement of the 4′oxygen atom of the sugar unit with a sulfur.

In some embodiments the oligonucleotide aptamer comprises a Xeno nucleic acid. X-aptamers and Xeno nucleic acids (XNAs) utilise unnatural or synthetic nucleotides to increase the chemical diversity and improve the binding characteristics of the aptamer. Xeno nucleic acids exhibit structural chemical changes in the sugar backbone moieties of the aptamer. Non-limiting examples of XNAs include 1,5-anhydrohexitol nucleic acid (HNA), Cyclohexene nucleic acid (CeNA), Threose nucleic acid (TNA), Glycol nucleic acid (GNA), Locked nucleic acid (LNA), Peptide nucleic acid (PNA) and FANA (Fluoro-Arabino nucleic acid) (Pinheiro and Holliger (2014) Trends in Biotechnology 32(6): 321-328). In addition to offering different chemical characteristics to provide increased chemical diversity for aptamer binding, XNAs are not susceptible to nuclease degradation.

In some embodiments the oligonucleotide aptamer comprises modifications on the nucleobase. The introduction of functionalities at the level of the nucleobase increase the contact interactions of oligonucleotides and their target molecule, and can generate additional secondary structures that are not accessible to wild-type nucleic acids. Non-limiting examples of modifications to nucleobases include modifications at the C5-position of the pyrimidines, and the N7 position of 7-deaza-purines. SOMamers (Slow off-rate modified aptamers), utilise distinct nucleobase modifications at the 5′ position of deoxyuridine, and are discussed below. As one skilled in the art would appreciate, oligonucleotide aptamers of the invention encompass the functional features defined for the genus of aptamers discussed herein to which oligonucleotide aptamers belong.

### Gasdermin D Specific SOMamers

In some embodiments the aptamer is non-proteinaceous. In some embodiments the aptamer is a SOMamer. SOMamers or (Slow off-rate modified aptamers) are a next generation aptamer platform that overcome some of the limitations of traditional oligonucleotide aptamers (Rohloff et al., (2014) Molecular Therapy Nucleic Acids 3, e201). They are short single-stranded oligonucleotides that are selected in vitro from large randomized libraries to bind a molecular target. As discussed above the chemical diversity of nucleic acid libraries is lower compared with protein based libraries, and the nucleic acid bases have a smaller range of physicochemical properties compared to amino acids. The introduction of non-native functional groups into nucleic acid bases therefore provides a method to increase the chemical diversity of the nucleic acid, and therefore improve the dissociation constant of the aptamer to bind to a range of molecular targets. SOMamer reagents have been developed to increase the chemical diversity of the aptamer. The functional groups that improve nucleic acid aptamers for protein binding typically have hydrophobic aromatic character. The modification of SOMamers are engineered with deoxyuridine residues that have been functionalized at the 5-position with different protein like moieties. Non-limiting examples of the protein like moieties include, 3-indolyl-carboxamide; Bn, benzyl; Pe, 2-phenylethyl; Pp, 3-phenylpropyl; Th, 2-thiophenylmethyl; FBn, 4-fluorobenzyl; Nap, 1-naphthylmethyl; 2Nap, 2-naphthylmethyl; Ne, 1-naphthyl-2-ethyl; 2Ne, 2-naphthyl-2-ethyl; Trp, 3-indole-2-ethyl; Bt, 3-benzothiophenyl-2-ethyl; Bf, 3-benzofuranyl-2-ethyl; Bi, 1-benzimidazol-2-ethyl; Tyr, 4-hydroxyphenyl-2-ethyl; Pyr, 4-pyridinylmethyl; MBn, 3,4-methylenedioxybenzyl; MPe, 3,4-methylenedioxyphenyl-2-ethyl; 3MBn, 3-methoxybenzyl; 4MBn, 4-methoxybenzyl; 3,4MBn, 3,4-dimethoxybenzyl; RTHF, R-tetrahydrofuranylmethyl; STHF, S-tetrahydrofuranylmethyl; Moe, morpholino-2-ethyl; Thr, R-2-hydroxypropyl; and iBu, iso-butyl groups (Rohloff et al., Molecular Therapy-Nucleic acids (2014) 3e201). In addition the functionalization of the nucleic acid improves the nuclease resistance of the aptamer. In some embodiments the target molecule used in the selection process of the SOMamer is SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the target molecule is SEQ ID NO: 9. As one skilled in the art would appreciate, SOMamers of the invention encompass the functional features defined for the genus of aptamers discussed herein to which SOMamers belong.

### Gasdermin D Specific Peptide Aptamers

In some embodiments the aptamer is a peptide aptamer. Peptide aptamers are small proteins that are selected to bind to specific target molecules. Peptide aptamers are characterised by high stability, high solubility, and fast folding kinetics (Reverdatto et al., Curr Top Med Chem (2015) 15(2) 1082-1101). They are defined as a short amino acid sequence that is able to bind to a target molecule. Peptide aptamers are essentially a ‘loop on a scaffold’, wherein the loop provides the peptide region that binds to the target molecule, and the scaffold is the protein backbone on which the loop is grafted. The presence of the loop on a ‘scaffold’ induces a conformational constraint on the loop region, stabilizes the insert loop, and makes it more likely to fold and recognize the target molecule. The binding affinity of such constrained peptide aptamers is often higher than the free peptide loop due to the lower conformational entropy of the restricted peptide loop. One skilled in the art would appreciate that the peptide loop aptamer could be grafted onto the scaffold structure of the antigen binding proteins discussed above or can be incorporated into existing secondary structural elements of the antigen binding proteins discussed above. Peptide aptamers often display a smaller binding footprint than nucleic acid aptamers.

Peptide aptamers can be selected by high throughput screening of large combinatorial peptide libraries. Screening the combinatorial libraries can be accomplished by extracellular display of the combinatorial library and screening for binding to the target molecule, for example through methods that are routine in the art. Non-limiting examples for the selection of peptide aptamers include: phage display, cell surface display in bacteria (such as Escherichia coli or Staphylococcus), cell surface display in yeast, cell surface display in eukaryotic cells (such as HEK293 cells), ribosome display, mRNA display, DNA display and in vitro compartmentalization. Alternatively intracellular selection methods include protein fragment complementation assays, like yeast two hybrid assays (Y2H). In some embodiments the target molecule used in the selection of the peptide aptamer is SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the target molecule is SEQ ID NO: 9. As one skilled in the art would appreciate, peptide aptamers of the invention encompass the functional features defined for the genus of aptamers discussed herein to which oligonucleotide aptamers belong.

Methods of Using Inhibitors of Gasdermin D

The invention provides a method for inhibiting an activity and/or function of gasdermin D, comprising contacting the gasdermin D inhibitor of the invention with gasdermin D.

The invention provides a method for neutralizing an activity and/or function of gasdermin D, comprising contacting the gasdermin D inhibitor of the invention, with gasdermin D.

The invention also provides use of the gasdermin D inhibitor of the invention in inhibiting an activity and/or function of gasdermin D.

The invention also provides use of the gasdermin D inhibitor of the invention in neutralizing an activity and/or function of gasdermin D.

In a preferred embodiment the gasdermin D inhibitor is an antigen binding protein.

In a preferred embodiment, the gasdermin D inhibitor is an antibody or antigen binding fragment thereof.

In a preferred embodiment, the gasdermin D inhibitor is an aptamer.

In some embodiments the activity and/or function of gasdermin D includes any biological effect of gasdermin D. In some embodiments, the activity and/or function of gasdermin D includes the ability of gasdermin D to interact or bind to another protein. In some embodiments, the activity and/or function of gasdermin D includes the ability of gasdermin D to interact or bind to another molecule of gasdermin D. In some embodiments, the activity and/or function of gasdermin D includes the ability of gasdermin D to associate with lipids. In some embodiments, the activity and/or function of gasdermin D includes the ability of gasdermin D to associate with phosphatidylinositol 4-phosphate and/or phosphatidylinositol 4,5-bisphosphate. In some embodiments, the activity and/or function of gasdermin D includes the ability of gasdermin D to insert into the cell membrane. In some embodiments, the activity and/or function of gasdermin D includes the ability of gasdermin D to oligomerise with other molecules of gasdermin D. In some embodiments, the activity and/or function of gasdermin D includes the ability of gasdermin D to form protein-protein interactions between gasdermin D subunits. In some embodiments, the activity and/or function of gasdermin D includes the ability of gasdermin D to form a multimeric pore.

In some embodiments, the activity and/or function of gasdermin D includes any activity that results from the formation of a gasdermin D multimeric pore. In some embodiments, the activity and/or function of gasdermin D includes dissipation of cellular ion gradients due to pore formation. In some embodiments, the activity and/or function of gasdermin D includes the extracellular release of inflammatory cytokines due to pore formation. In some embodiments the activity and/or function of gasdermin D is the release of IL-1β and/or IL-18. In some embodiments, the activity and/or function of gasdermin D includes the osmotic lysis of the cell due to pore formation. In some embodiments, the activity and/or function of gasdermin D includes cell death induced by pyroptosis due to pore formation. In some embodiments, the activity and/or function of gasdermin D includes the larger inflammatory response that results upon proinflammatory pyroptotic cell death.

The invention provides a method for inhibiting and/or neutralizing an activity and/or function of gasdermin D, comprising contacting a gasdermin D inhibitor with gasdermin D; wherein the gasdermin D inhibitor binds to gasdermin D and inhibits gasdermin D, such as wherein the inhibitor binds to gasdermin D and neutralizes gasdermin D, wherein the inhibition and/or neutralization of gasdermin D is the inhibition and/or neutralization of:
i) an activity of gasdermin D, such as wherein the inhibitor neutralizes an activity of gasdermin D; and/or
ii) a function of gasdermin D, such as wherein the inhibitor neutralizes a function of gasdermin D;
   and wherein:
a) the inhibitor is an extracellular inhibitor;
b) the inhibitor binds to the N-terminal domain of gasdermin D;
c) the inhibitor binds to an epitope comprising SEQ ID NO: 9; and
d) the inhibitor binds to SEQ ID NO: 9.

The invention provides use of a gasdermin D inhibitor in inhibiting and/or neutralizing an activity and/or function of gasdermin D; wherein the gasdermin D inhibitor binds to gasdermin D and inhibits gasdermin D, such as wherein the inhibitor binds to gasdermin D and neutralizes gasdermin D, wherein the inhibition and/or neutralization of gasdermin D is the inhibition and/or neutralization of:
i) an activity of gasdermin D, such as wherein the inhibitor neutralizes an activity of gasdermin D; and/or
ii) a function of gasdermin D, such as wherein the inhibitor neutralizes a function of gasdermin D;
   and wherein:
a) the inhibitor is an extracellular inhibitor;
b) the inhibitor binds to the N-terminal domain of gasdermin D;
c) the inhibitor binds to an epitope comprising SEQ ID NO: 9; and
d) the inhibitor binds to SEQ ID NO: 9.

Sometimes, the inhibitor is proteinaceous, for example an antigen binding protein, such as wherein the antigen binding protein is an antibody or an antigen binding fragment thereof.

Therapeutic Uses of Inhibitors of Gasdermin D

Gasdermin D has been implicated in a number of diseases including: sepsis (Kayagaki et al., Nature (2015) 526(7575): 666-671 and Wu et al., Immunity (2019) 50(6) 1401-1411), non-alcoholic steatohepatitis (Xu et al., Journal of Heaptology (2018) 68(4): 773-782), lung cancer (Gao et al., Oncology Reports 40(4):1971-1984), familial Mediterranean Fever (Kanneganti et al., Journal of Experimental Medicine (2018) 215(6): 1519-1529), autoinflammatory diseases such as cryopyrin-associated periodic syndromes (CAPS) (Xiao et al., PloS Biology (2018) 16(11)), non-alcoholic fatty liver disease, Alzheimer's disease, Parkinson's disease, age related macular degeneration, atherosclerosis, asthma and allergy airway inflammation, gout, Crohn's, ulcerative colitis, inflammatory bowel disease, hypertension, nephropathy, myocardial infarction, multiple sclerosis, experimental autoimmune encephalitis, hyperinflammation following influenza infection, graft-versus-host disease, stroke, silicosis, asbestosis, mesothelioma, type 1 diabetes, type 2 diabetes, obesity-induced inflammation, insulin resistance, rheumatoid arthritis, myelodysplastic syndrome, contact hypersensitivity, joint inflammation triggered by chikungunya virus and traumatic brain injury (Mangan et al., (2018) Nature Rev Drug Discov 17, 588-606). The inhibition of gasdermin D activity and/or function is therefore of therapeutic use.

Accordingly, the invention provides an inhibitor for use in inhibiting gasdermin D, such as neutralizing gasdermin D.

The invention also provides an inhibitor for use in neutralizing gasdermin D.

The invention also provides an inhibitor for use in inhibiting gasdermin D, wherein the inhibition of gasdermin D is the inhibition of an activity of gasdermin D, such as wherein the inhibitor neutralizes an activity of gasdermin D and/or the inhibition of gasdermin D is the inhibition of a function of gasdermin D, such as wherein the inhibitor neutralizes a function of gasdermin D.

The invention also provides an inhibitor for use in neutralizing gasdermin D, wherein the neutralization of gasdermin D is the neutralization of an activity of gasdermin D and/or the neutralization of gasdermin D is the neutralization of a function of gasdermin D.

The invention also provides an inhibitor for use in therapy.

In some embodiments, the inhibitor is used in the treatment of a disease caused by gasdermin D.

In some embodiments, the inhibitor is for use in the treatment of an indication selected from: sepsis, septic shock, non-alcoholic steatohepatitis, lung cancer, Familial Mediterranean Fever, autoinflammatory diseases, Cryoprin associated periodic syndromes, non-alcoholic fatty liver disease, Alzheimer's disease, Parkinson's disease, age related macular degeneration, atherosclerosis, asthma and allergy airway inflammation, gout, Crohn's, ulcerative colitis, inflammatory bowel disease, hypertension, nephropathy, myocardial infarction, multiple sclerosis, experimental autoimmune encephalitis, hyperinflammation following influenza infection, graft versus host disease, stroke, silicosis, asbestosis, mesothelioma, type 1 diabetes, type 2 diabetes, obesity-induced inflammation, insulin resistance, rheumatoid arthritis, myelodysplastic syndrome, contact hypersensitivity, joint inflammation triggered by chikungunya virus and traumatic brain injury.

In a preferred embodiment, the inhibitor is an antigen binding protein that binds to gasdermin D.

In a preferred embodiment, the inhibitor is an antibody or antigen binding fragment thereof that binds to gasdermin D.

In a preferred embodiment, the inhibitor is an aptamer that binds to gasdermin D.

The terms “treating” or “treatment” refer to any success or indicia of success in the attenuation or amelioration of an injury, pathology or condition, including any objective or subjective parameter such as abatement, remission, diminishing of symptoms or making the condition more tolerable to the patient, slowing in the rate of degeneration or decline, making the final point of degeneration less debilitating, improving a subject's physical or mental well-being, or prolonging the length of survival. The treatment may be assessed by objective or subjective parameters; including the results of a physical examination, neurological examination, or psychiatric evaluations.

An “effective amount” or “therapeutically effective amount” refers to an amount effective, at dosages and for periods of time necessary, to achieve a desired therapeutic result. A therapeutically effective amount of a gasdermin D inhibitor may vary according to factors such as the disease state, age, sex, and weight of the individual, and the ability of the antibody to elicit a desired response in the individual. A therapeutically effective amount is also one in which any toxic or detrimental effects of the antibody or antibody portion are outweighed by the therapeutically beneficial effects.

The inhibitors of the invention described herein may aso be administered in combination therapy, i.e. combined with other therapeutic agents relevant for the disease or condition to be treated. Accordingly, in one embodiment, the inhibitor of the invention is for combination with one or more further therapeutic agents. Such combined administration may be simultaneous, separate or sequential, in any order. For simultaneous administration the agents may be administered as one composition or as separate compositions as appropriate.

The invention provides a gasdermin D inhibitor that binds to gasdermin D and inhibits gasdermin D, such as wherein the inhibitor binds to gasdermin D and neutralizes gasdermin D, wherein the inhibition and/or neutralization of gasdermin D is the inhibition and/or neutralization of:
i) an activity of gasdermin D, such as wherein the inhibitor neutralizes an activity of gasdermin D; and/or
ii) a function of gasdermin D, such as wherein the inhibitor neutralizes a function of gasdermin D;
   and wherein:
a) the inhibitor is an extracellular inhibitor;
b) the inhibitor binds to the N-terminal domain of gasdermin D;
c) the inhibitor binds to an epitope comprising SEQ ID NO: 9; and
d) the inhibitor binds to SEQ ID NO: 9;
for use in therapy.

The invention provides a gasdermin D inhibitor that binds to gasdermin D and inhibits gasdermin D, such as wherein the inhibitor binds to gasdermin D and neutralizes gasdermin D, wherein the inhibition and/or neutralization of gasdermin D is the inhibition and/or neutralization of:
i) an activity of gasdermin D, such as wherein the inhibitor neutralizes an activity of gasdermin D; and/or
ii) a function of gasdermin D, such as wherein the inhibitor neutralizes a function of gasdermin D;
   and wherein:
a) the inhibitor is an extracellular inhibitor;
b) the inhibitor binds to the N-terminal domain of gasdermin D;
c) the inhibitor binds to an epitope comprising SEQ ID NO: 9; and
d) the inhibitor binds to SEQ ID NO: 9;
   for use in the treatment of an indication selected from: sepsis, septic shock, non-alcoholic steatohepatitis, lung cancer, Familial Mediterranean Fever, autoinflammatory diseases, Cryoprin associated periodic syndromes, non-alcoholic fatty liver disease, Alzheimer's disease, Parkinson's disease, age related macular degeneration, atherosclerosis, asthma and allergy airway inflammation, gout, Crohn's, ulcerative colitis, inflammatory bowel disease, hypertension, nephropathy, myocardial infarction, multiple sclerosis, experimental autoimmune encephalitis, hyperinflammation following influenza infection, graft versus host disease, stroke, silicosis, asbestosis, mesothelioma, type 1 diabetes, type 2 diabetes, obesity-induced inflammation, insulin resistance, rheumatoid arthritis, myelodysplastic syndrome, contact hypersensitivity, joint inflammation triggered by chikungunya virus and traumatic brain injury.

Sometimes, the inhibitor is proteinaceous, for example an antigen binding protein, such as wherein the antigen binding protein is an antibody or an antigen binding fragment thereof.

Compositions

The pharmaceutical compositions provided herein comprise: a) an effective amount of inhibitor of the invention, such as the antibody, antibody fragment or antibody conjugate of the present invention, and b) a pharmaceutically acceptable carrier, which may be inert or physiologically active. As used herein, the term “pharmaceutically acceptable carriers” includes any and all solvents, dispersion media, coatings, antibacterial and antifungal agents, and the like that are physiologically compatible. Examples of suitable carriers, diluents and/or excipients include one or more of water, saline, phosphate buffered saline, dextrose, glycerol, ethanol, and the like, as well as any combination thereof. In many cases, it will be preferable to include isotonic agents, such as sugars, polyalcohols, or sodium chloride in the composition. In particular, relevant examples of suitable carrier include: (1) Dulbecco's phosphate buffered saline, pH.about.7.4, containing or not containing about 1 mg/mL to 25 mg/mL human serum albumin, (2) 0.9% saline (0.9% w/v sodium chloride (NaCl)), and (3) 5% (w/v) dextrose; and may also contain an antioxidant such as tryptamine and a stabilizing agent such as Tween 20®.

The compositions herein may also contain a further therapeutic agent, as necessary for the particular disorder being treated. Preferably, the inhibitor of the invention and the supplementary active compound will have complementary activities that do not adversely affect each other. In a preferred embodiment, the further therapeutic agent is cytarabine, an anthracycline, histamine dihydrochloride, or interleukin 2. In a preferred embodiment, the further therapeutic agent is a chemotherapeutic agent.

The compositions of the invention may be in a variety of forms. These include for example liquid, semi-solid, and solid dosage forms, but the preferred form depends on the intended mode of administration and therapeutic application. Typical preferred compositions are in the form of injectable or infusible solutions. The preferred mode of administration is parenteral (e.g. intravenous, intramuscular, intraperinoneal, subcutaneous). In a preferred embodiment, the compositions of the invention are administered intravenously as a bolus or by continuous infusion over a period of time. In another preferred embodiment, they are injected by intramuscular, subcutaneous, intra-articular, intrasynovial, intratumoral, peritumoral, intralesional, or perilesional routes, to exert local as well as systemic therapeutic effects.

Sterile compositions for parenteral administration can be prepared by incorporating the inhibitor of the invention, such as the antibody, antibody fragment or antibody conjugate of the present invention, in the required amount in the appropriate solvent, followed by sterilization by microfiltration. As solvent or vehicle, there may be used water, saline, phosphate buffered saline, dextrose, glycerol, ethanol, and the like, as well as combination thereof. In many cases, it will be preferable to include isotonic agents, such as sugars, polyalcohols, or sodium chloride in the composition. These compositions may also contain adjuvants, in particular wetting, isotonizing, emulsifying, dispersing and stabilizing agents. Sterile compositions for parenteral administration may also be prepared in the form of sterile solid compositions which may be dissolved at the time of use in sterile water or any other injectable sterile medium.

The inhibitor of the invention, such as the antibody, antibody fragment or antibody conjugate of the present invention, may also be orally administered. As solid compositions for oral administration, tablets, pills, powders (gelatine capsules, sachets) or granules may be used. In these compositions, the active ingredient according to the invention is mixed with one or more inert diluents, such as starch, cellulose, sucrose, lactose or silica, under an argon stream. These compositions may also comprise substances other than diluents, for example one or more lubricants such as magnesium stearate or talc, a coloring, a coating (sugar-coated tablet) or a glaze.

As liquid compositions for oral administration, there may be used pharmaceutically acceptable solutions, suspensions, emulsions, syrups and elixirs containing inert diluents such as water, ethanol, glycerol, vegetable oils or paraffin oil. These compositions may comprise substances other than diluents, for example wetting, sweetening, thickening, flavoring or stabilizing products.

Kits

Also provided herein are includes kits, e.g., comprising a described inhibitor, and instructions for the use of the inhibitor. In preferred embodiments, the inhibitor is an inhibitor of gasdermin D. In a preferred embodiment the inhibitor is an antigen binding protein that binds to gasdermin D. In a preferred embodiment the inhibitor is an antibody or antibody binding fragment thereof that binds to gasdermin D. In a preferred embodiment the inhibitor is an aptamer that binds to gasdermin D. The instructions may include directions for using the inhibitor in vitro, in vivo or ex vivo.

Typically, the kit will have a compartment containing inhibitor. The inhibitor may be in a lyophilized form, liquid form, or other form amendable to being included in a kit. The kit may also contain additional elements needed to practice the method described on the instructions in the kit, such a sterilized solution for reconstituting a lyophilized powder, additional agents for combining with the inhibitor prior to administering to a patient, and tools that aid in administering the inhibitor to a patient.

### Other Regions of Interest of Gasdermin D

The crystal structure of the N-terminal domain of a murine gasdermin protein, together with mutagenesis and homology modelling, has aided in the identification of key residues of the protein that are involved in lipid binding and oligomerisation. Residues in the α1 helix and the adjacent β1-β2 loop, together with the residues F49, W50, K51, R53, R137, K145, R151 and R153 have been implicated in lipid binding (Liu et al., Immunity 13 May 2019).

One skilled in the art would also appreciate that oligomerisation requires protein-protein interactions between multiple subunits of gasdermin D. Inhibitors that can bind to the protein-protein interaction interface therefore can have the capacity to inhibit gasdermin D oligomerisation. Key structural regions that have been implicated in the interface interactions between gasdermin D subunits include the α1′ helix, α2 helix, α3 helix, and the β2 strand, β3 strand and β11 strand.

In addition a number of individual residues have been implicated in the mediation of oligomerisation, in particular: L28, C38, L59, F80, 190, V94, C191, L192, V229, L230, L231, and F232.

Accordingly in some embodiments inhibitors as defined above bind to an epitope comprising one or more of SEQ ID NOs: 10, SEQ ID NO: 11, SEQ ID NO: 12, SEQ ID NO: 13, SEQ ID NO: 14, SEQ ID NO: 15, or SEQ ID NO: 16, such as one or more of SEQ ID NOs: 10, SEQ ID NO: 11, SEQ ID NO: 12, SEQ ID NO: 13, SEQ ID NO: 14, SEQ ID NO: 15, or SEQ ID NO: 16.

### Methods for Assessing Gasdermin D Inhibitors

The activity of the inhibitors discussed herein can be assessed using in vitro methods.

One such method relies on measurement of the inhibitors' inhibition of the lysis of Human THP-1 cells (other methods rely on measurement of the inhibtors' inhibition of the lysis of primary human monocytes, human macrophages, and include primary blood-derived monocytes and macrophages differentiated ex vivo). Human THP-1 cells are an immortalized monocyte-like cell line that are derived from the peripheral blood of acute monocytic leukaemia (M5 subtype). In this assay THP-1 cells are cultured in RPMI medium supplemented with 10% FCS, sodium pyruvate and the antibiotics penicillin and streptomycin. Under maintenance/expansion culturing conditions these cells grow in suspension, however after overnight differentiation using phorbol 12-myristate 13-acetate these cells can be matured such that they become adherent. After this differentiation, the NLRP3 inflammasome can be activated by stimulation with nigericin. Unchecked, this will lead to fast cell death induction (typically observed within 1-2 hours). This fast cell death induction (pyroptosis) is driven by GSDMD pore formation. Pyroptosis is a lytic form of cell death and thus can be monitored by measuring the increase in fluorescent signal of DNA dyes that cannot cross an intact cell membrane of live cells. In these examples SYTOX™ green is the dye that is used for detection, but the working principle is identical to the use of compounds like Propidium Iodide in FACS.

Accordingly, sometimes the inhibition of gasdermin D is determined by:

(i) culturing THP-1 cells in RPMI medium supplemented with 10% FCS, sodium pyruvate and the antibiotics penicillin and streptomycin;
(ii) adding phorbol 12-myristate 13-acetate (PMA) to a concentration of 100 ng/ml and incubating for 16 hours;
(iii) replacing the medium with RPMI medium supplemented with 10% FCS, sodium pyruvate and the antibiotics penicillin and streptomycin, and fluorescent nucleic acid binding dye (e.g. SYTOX™ green; at 1 μM final concentration) and incubating for 2 hours;
(iv) adding inhibitor to the cells and incubating with the inhibitor for 30 minutes;
(v) adding nigericin, at a concentration of 20 μM, to the cells; and
(vi) recording cell death as indicated by fluorescence of cells.

IL-1β and IL-18 release can also be measured using a modified form of this assay. For example, at a defined timepoint after nigericin stimulation, such as 80 minutes after nigericin stimulation, the supernatant of the assay is harvested and the IL-1β and IL-18 released into the supernatant quantified. Quantification can be achieved using the Luminex assay system or the Meso Scale Discovery (MSD) assay system according to the manufacturer's instructions.

Sometimes wherein a THP-1 cell assay is mentioned above, it is possible to use alternative methods for measuring activity.

One such method relies on measurement of the inhibitors' inhibition of the release of IL-1β from primary human monocytes isolated from fresh human blood PBMCs. This method is conceptually similar to the THP-1 assay, but uses a different cell type. The cells are isolated from fresh human blood PBMCs, primed with lipopolysaccharide (LPS), and the NLRP3 inflammasome is activated by stimulation with nigericin. This leads to the formation of the GSDMD pore and release of IL-1β. At a defined time point after nigericin stimulation, such as 1 hour after nigericin stimulation, the supernatant of the assay is harvested and the IL-1β release into the supernatant can be quantified. Quantification of IL-1β release can be achieved using the Luminex assay system or the Meso Scale Discovery (MSD) assay system according to the manufacturer's instructions.

Accordingly, sometimes the inhibition of gasdermin D is determined by:

(i) isolating primary human monocytes (CD14+) from fresh human blood PBMCs using CD14 microbeads;
(ii) washing the cells twice with fresh medium (RPMI supplemented with 10% FBS);
(iii) priming the cells with lipopolysaccharide (LPS) at a concentration of 100 ng/ml for 3 hours;
(iv) adding inhibitor to the cells and incubating with the inhibitor for 15 minutes;
(v) adding nigericin, at a concentration of 15 μM to the cells; and
(vi) harvesting the supernatant and measuring IL-1β release.

Another such method relies on measurement of the inhibitors' inhibition of the cell death and IL-1β release in a murine cell line. This method is conceptually similar to the THP-1 assay and can be used when the inhibitor inhibits murine GSDMD. Gasdermin D is activated by both the NLRP3 and NLRP1B inflammasomes. Balb/C mice express an anthrax lethal toxin (LeTx) sensitive NLRP1B allele. Incubation of the murine cell line with LeTx therefore allows for the specific activation of the NLRP1B inflammasome. Activation of either the NLRP3 or NLRP1B inflammasome leads to the induction of cell death and release of IL-1β due to gasdermin D pore formation. Pyroptosis of the murine cells can thus can be monitored by measuring the increase in fluorescent signal of DNA dyes that cannot cross an intact cell membrane of live cells. In these examples SYTOX™ green is the dye that is used for detection, but the working principle is identical to the use of compounds like Propidium Iodide in FACS. In addition, quantification of IL-1β release can be achieved using the Luminex assay system or the Meso Scale Discovery (MSD) assay system according to the manufacturer's instructions.

Accordingly, sometimes the inhibition of gasdermin D is determined by:

(i) isolating bone marrow from the tibia and femur of Balb/C mice;
(ii) differentiating the bone marrow into bone marrow derived macrophages (BMDM) by culturing the cells for 6 days in IMDM medium supplemented with 10% FBS and penicillin/streptomycin, mixed with L929 culture supernatant;
(iii) washing, collecting and plating the cells in fresh IMDM medium supplemented with 10% FBS and penicillin/streptomycin, for example at a density of 50,000 cells per well, and allowing the cells to adhere overnight;
(iv) washing the adherent cells and replacing the medium with fresh IMDM medium supplemented with 10% FBS, penicillin/streptomycin and fluorescent nucleic acid binding dye (e.g. SYTOX™ green; at a concentration of 1 μM);
(v) priming the cells with lipopolysaccharide (LPS) at a concentration of 100 ng/ml for 3 hours;
(vi) adding inhibitor to the cells and incubating with the inhibitor for 15 minutes;
(vii) adding nigericin (15 μM), to the cells to activate the NLRP3 inflammasome, or adding LeTX (1 μg/ml protective antigen (PA)+0.5 μg/ml lethal factor (LF)) to activate the NLRP1B inflammasome, for an additional 1.5 hours; and
(viii) recording cell death as indicated by fluorescence of cells and/or harvesting the supernatant of the cells and quantifying IL-1β release.

### General

Various terms relating to aspects of the description are used throughout the specification and claims. Such terms are to be given their ordinary meaning in the art unless otherwise indicated. Other specifically defined terms are to be construed in a manner consistent with the definitions provided herein.

As used in this specification and the appended claims, the singular forms “a,” “an,” and “the” include plural referents unless the content clearly dictates otherwise. Thus, for example, reference to “a cell” includes a combination of two or more cells, and the like.

The term “about” as used herein when referring to a measurable value such as an amount, a temporal duration, and the like, is meant to encompass variations of up to ±5% from the specified value, as such variations are appropriate to perform the disclosed methods. Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

“Isolated” means a biological component (such as a nucleic acid, peptide or protein) has been substantially separated, produced apart from, or purified away from other biological components of the organism in which the component naturally occurs, i.e., other chromosomal and extrachromosomal DNA and RNA, and proteins. Nucleic acids, peptides and proteins that have been “isolated” thus include nucleic acids and proteins purified by standard purification methods. “Isolated” nucleic acids, peptides and proteins can be part of a composition and still be isolated if such composition is not part of the native environment of the nucleic acid, peptide, or protein. The term also embraces nucleic acids, peptides and proteins prepared by recombinant expression in a host cell as well as chemically synthesized nucleic acids. An “isolated” antibody or antigen-binding fragment, as used herein, is intended to refer to an antibody or antigen-binding fragment which is substantially free of other antibodies or antigen-binding fragments having different antigenic specificities (for instance, an isolated antibody that specifically binds to gasdermin D is substantially free of antibodies that specifically bind antigens other than gasdermin D). An isolated antibody that specifically binds to an epitope, isoform or variant of gasdermin D may, however, have cross-reactivity to other related antigens, for instance from other species (such as gasdermin D species homologs).

“Polynucleotide,” synonymously referred to as “nucleic acid molecule,” “nucleotides” or “nucleic acids,” refers to any polyribonucleotide or polydeoxyribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications may be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

The terms “polypeptide” or “protein” means a macromolecule having the amino acid sequence of a native protein, that is, a protein produced by a naturally-occurring and non-recombinant cell; or it is produced by a genetically-engineered or recombinant cell, and comprise molecules having the amino acid sequence of the native protein, or molecules having deletions from, additions to, and/or substitutions of one or more amino acids of the native sequence. The term also includes amino acid polymers in which one or more amino acids are chemical analogs of a corresponding naturally-occurring amino acid and polymers. The term “polypeptide fragment” refers to a polypeptide that has an amino-terminal deletion, a carboxyl-terminal deletion, and/or an internal deletion as compared with the full-length native protein. Such fragments can also contain modified amino acids as compared with the native protein. In certain embodiments, fragments are about five to 500 amino acids long. For example, fragments can be at least 5, 6, 8, 10, 14, 20, 50, 70, 100, 110, 150, 200, 250, 300, 350, 400, or 450 amino acids long. Useful polypeptide fragments include immunologically functional fragments of antibodies, including binding domains.

The term “epitope” means a protein determinant capable of specific binding to an inhibitor. Epitopes usually consist of surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents. The epitope may comprise amino acid residues directly involved in the binding and other amino acid residues, which are not directly involved in the binding, such as amino acid residues which are effectively blocked or covered by the specifically antigen binding peptide (in other words, the amino acid residue is within the footprint of the specifically antigen binding peptide).

The term “vector” means any molecule or entity (e.g., nucleic acid, plasmid, bacteriophage or virus) used to transfer protein coding information into a host cell.

The term “expression vector” or “expression construct” refers to a vector that is suitable for transformation of a host cell and contains nucleic acid sequences that direct and/or control (in conjunction with the host cell) expression of one or more heterologous coding regions operatively linked thereto. An expression construct can include, but is not limited to, sequences that affect or control transcription, translation, and, if introns are present, affect RNA splicing of a coding region operably linked thereto.

As used herein, “operably linked” means that the components to which the term is applied are in a relationship that allows them to carry out their inherent functions under suitable conditions. For example, a control sequence in a vector that is “operably linked” to a protein coding sequence is ligated thereto so that expression of the protein coding sequence is achieved under conditions compatible with the transcriptional activity of the control sequences.

The term “host cell” means a cell that has been transformed, or is capable of being transformed, with a nucleic acid sequence and thereby expresses a gene of interest. The term includes the progeny of the parent cell, whether or not the progeny is identical in morphology or in genetic make-up to the original parent cell, so long as the gene of interest is present.

The term “transfection” means the uptake of foreign or exogenous DNA by a cell, and a cell has been “transfected” when the exogenous DNA has been introduced inside the cell membrane. A number of transfection techniques are well known in the art and are disclosed herein. See, e.g., Graham et al., 1973, Virology 52:456; Sambrook et al., 2001, Molecular Cloning: A Laboratory Manual, supra; Davis et al., 1986, Basic Methods in Molecular Biology, Elsevier; Chu et al, 1981, Gene 13:197. Such techniques can be used to introduce one or more exogenous DNA moieties into suitable host cells.

Standard techniques can be used for recombinant DNA, oligonucleotide synthesis, and tissue culture and transformation (e.g., electroporation, lipofection). Enzymatic reactions and purification techniques can be performed according to manufacturer's specifications or as commonly accomplished in the art or as described herein. The foregoing techniques and procedures can be generally performed according to conventional methods well known in the art and as described in various general and more specific references that are cited and discussed throughout the present specification. See, e.g., Sambrook et al, Molecular Cloning: A Laboratory Manual (2d ed., Cold Spring Harbor Laboratory Press, Cold Spring Harbor, N. Y. (1989. Unless specific definitions are provided, the nomenclatures utilized in connection with, and the laboratory procedures and techniques of, analytical chemistry, synthetic organic chemistry, and medicinal and pharmaceutical chemistry described herein are those well known and commonly used in the art. Standard techniques can be used for chemical syntheses, chemical analyses, pharmaceutical preparation, formulation, and delivery, and treatment of patients.

The term “gasdermin D” or “GSDMD” includes a polypeptide as set forth in SEQ ID NO: 1 or fragments thereof, as well as related polypeptides, which include, but are not limited to, allelic variants, splice variants, derivative variants, substitution variants, deletion variants, and/or insertion variants including the addition of an N-terminal methionine, fusion polypeptides, and interspecies homologs. In certain embodiments, a gasdermin D polypeptide includes terminal residues, such as, but not limited to, leader sequence residues, targeting residues, amino terminal methionine residues, lysine residues, tag residues and/or fusion protein residues. The term “gasdermin D” denotes both the full length gasdermin D and the product generated following protease cleavage. “GSDMD^{Nterm}” refers to the N-terminal domain of gasdermin D and includes a polypeptide as set forth in SEQ ID NO: 2, for example wherein the N-terminal domain has the sequence of SEQ ID NO: 2. “GSDMD^{Cterm}” refers to the C-terminal domain of gasdermin D and includes a polypeptide as set forth in SEQ ID NO: 3, for example wherein the C-terminal domain has the sequence of SEQ ID NO: 3.

The term “gasdermin D activity” and/or “gasdermin D function” includes any biological effect of gasdermin D. In some embodiments, the term includes the ability of gasdermin D to interact or bind to another protein. In some embodiments, the term includes the ability of gasdermin D to interact or bind to another molecule of gasdermin D. In some embodiments, the term includes the ability of gasdermin D to associate with lipids. In some embodiments, the term includes the ability of gasdermin D to associate with phosphatidylinositol 4-phosphate and/or phosphatidylinositol 4,5-bisphosphate. In some embodiments, the term includes the ability of gasdermin D to insert into the cell membrane. In some embodiments, the term includes the ability of gasdermin D to oligomerise with other molecules of gasdermin D. In some embodiments, the term includes the ability of gasdermin D to form protein-protein interactions between gasdermin D subunits. In some embodiments, the term includes the ability of gasdermin D to form a multimeric pore.

In some embodiments, the term includes any activity that results from the formation of a gasdermin D multimeric pore. In some embodiments, the term includes dissipation of cellular ion gradients due to pore formation. In some embodiments, the term includes the extracellular release of inflammatory cytokines due to pore formation. In some embodiments the term includes the release of IL-1β and/or IL-18. In some embodiments, the term includes the osmotic lysis of the cell due to pore formation. In some embodiments, the term includes cell death induced by pyroptosis due to pore formation. In some embodiments, the term includes the larger inflammatory response that results upon proinflammatory pyroptotic cell death.

Further embodiments of the present invention are listed below:
1. A gasdermin D inhibitor that binds to gasdermin D and inhibits gasdermin D, such as wherein the inhibitor binds to gasdermin D and neutralizes gasdermin D.
2. The gasdermin D inhibitor of embodiment 1, wherein the inhibition of gasdermin D is the inhibition of:
   i) an activity of gasdermin D, such as wherein the inhibitor neutralizes an activity of gasdermin D; and/or
   ii) a function of gasdermin D, such as wherein the inhibitor neutralizes a function of gasdermin D.
3. A gasdermin D inhibitor that binds to gasdermin D and neutralizes gasdermin D.
4. The gasdermin D inhibitor of embodiment 3, wherein the neutralization of gasdermin D is the neutralization of:
   i) an activity of gasdermin D; and/or
   ii) a function of gasdermin D.
5. The inhibitor of any one of embodiments 1 to 4, wherein the inhibitor is an extracellular inhibitor.
6. The inhibitor of any one of embodiments 1 to 5, wherein the inhibitor binds to gasdermin D on the cell surface.
7. The inhibitor of any one of embodiments 1 to 6, wherein the inhibitor binds to gasdermin D and does not cross the cell membrane, unless it is bound to gasdermin D.
8. The inhibitor of any one of embodiments 1 to 7, wherein the inhibitor is a large molecule.
9. The inhibitor of any one of embodiments 1 to 8, wherein the inhibitor has a molecular weight of >2 kDa, >3 kDa, >4 kDa, >5 kDa, >6 kDa, >7 kDa, >8 kDa, >9 kDa or >10 kDa.
10. The inhibitor of any one of embodiments 1 to 9, wherein the inhibitor binds to the N-terminal domain of gasdermin D.
11. The inhibitor of any one of embodiments 1 to 10, wherein the inhibitor binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9.
12. The inhibitor of embodiment 11, wherein the inhibitor binds to an epitope comprising SEQ ID NO: 9.
13. The inhibitor of any one of embodiments 1 to 12, wherein the inhibitor binds to SEQ ID NO: 2, SEQ ID NO: 4, SEQ ID NO: 5, SEQ ID NO: 6, SEQ ID NO: 7, SEQ ID NO: 8, or SEQ ID NO: 9.
14. The inhibitor of embodiment 13, wherein the inhibitor binds to SEQ ID NO: 9.
15. The inhibitor of any one of embodiments 1 to 14, wherein the inhibitor binds to the N-terminal domain of gasdermin D and binds to an isolated peptide of SEQ ID NO: 2, SEQ ID NO: 4, SEQ ID NO: 5, SEQ ID NO: 6, SEQ ID NO: 7, SEQ ID NO: 8, or SEQ ID NO: 9.
16. The inhibitor of embodiment 15, wherein the inhibitor binds to the N-terminal domain of gasdermin D and binds to an isolated peptide of SEQ ID NO: 9.
17. The inhibitor of any one of embodiments 1 to 16, wherein the inhibitor binds to gasdermin D and inhibits its association with lipids, such as wherein the inhibitor binds to gasdermin D and neutralizes the association of gasdermin D with lipids.
18. The inhibitor of embodiment 17, wherein the inhibitor binds to gasdermin D and inhibits its association with phosphatidylinositol 4-phosphate and/or phosphatidylinositol 4,5-bisphosphate, such as wherein the inhibitor binds to gasdermin D and neutralizes the association of gasdermin D with phosphatidylinositol 4-phosphate and/or phosphatidylinositol 4,5-bisphosphate.
19. The inhibitor of any one of embodiments 1 to 18, wherein the inhibitor binds to gasdermin D and inhibits the oligomerisation of gasdermin D, such as wherein the inhibitor neutralizes oligomerisation of gasdermin D.
20. The inhibitor of embodiment 19, wherein the inhibitor inhibits protein-protein interactions between gasdermin D subunits, such as wherein the inhibitor neutralizes protein-protein interactions between gasdermin D subunits.
21. The inhibitor of any one of embodiments 1 to 18, wherein the inhibitor binds to a gasdermin D multimeric pore.
22. The inhibitor of embodiment 21, wherein the inhibitor blocks the pore.
23. The inhibitor of embodiment 21, wherein the inhibitor disrupts protein-protein interactions between gasdermin D subunits of the pore.
24. The inhibitor of any one of embodiments 21 to 23, wherein the inhibitor binds to a gasdermin D subunit of the multimeric pore.
25. The inhibitor of embodiment 24, wherein the inhibitor blocks the pore.
26. The inhibitor of embodiment 24, wherein the inhibitor disrupts protein-protein interactions between gasdermin D subunits of the pore.
27. The inhibitor of any one of embodiments 1 to 26, wherein the inhibitor inhibits the release of IL-1β and/or IL-18.
28. The inhibitor of any one of embodiments 1 to 27, wherein the inhibitor inhibits cell death induced by pyroptosis, such as wherein the inhibitor neutralizes cell death induced by pyroptosis.
29. The inhibitor of embodiment 28, wherein the inhibitor inhibits cell death induced by pyroptosis by at least 5%, at least 10%, at least 20%, at least 30%, at least 40%, at least 50%, at least 60%, at least 70%, at least 80%, at least 90%, or about 100%, such as 100%.
30. The inhibitor of embodiment 28 or embodiment 29, wherein when administered to human THP-1 cells, the inhibitor inhibits cell death induced by pyroptosis by at least 5%, at least 10%, at least 20%, at least 30%, at least 40%, at least 50%, at least 60%, at least 70%, at least 80%, at least 90%, or about 100%, such as 100%, wherein the THP-1 cells are at a cell density of 50,000 cells in 100 μl, wherein the THP-1 cells have been pre-treated with phorbol 12-myristate 13-acetate for 16 hours, wherein the THP-1 cells have been induced with 20 μM nigericin, and wherein cell death is measured after 1 hour.
31. The inhibitor of any one of embodiments 1 to 30, wherein the inhibitor delays cell death induced by pyroptosis by >0.1 hours, >0.2 hours, >0.5 hours, >0.75 hours, >1 hour, >2 hours, >3 hours, >4 hours, or >6 hours.
32. The inhibitor of embodiment 31, wherein when administered to human THP-1 cells the inhibitor delays cell death induced by pyroptosis by >0.1 hours, >0.2 hours, >0.5 hours, >0.75 hours, >1 hour, >2 hours, >3 hours, >4 hours, or >6 hours, wherein the THP-1 cells are at a cell density of 50,000 cells in 100 μl, wherein the THP-1 cells have been pre-treated with phorbol 12-myristate 13-acetate for 16 hours, and wherein the THP-1 cells have been induced with 20 μM nigericin.
33. The inhibitor of any one of embodiments 1 to 32, wherein the inhibitor cross reacts with an old world monkey gasdermin D or a new world monkey gasdermin D.
34. The inhibitor of any one of embodiments 1 to 33, wherein the inhibitor is proteinaceous, for example an antigen binding protein.
35. The antigen binding protein of embodiment 34, wherein the antigen binding protein is an antibody or an antigen binding fragment thereof, a multispecific antibody, a VHH domain, a VNAR domain, a VLR domain, a fibronectin type III domain, a centyrin, a kringle domain, a DARPin, a cysteine knot miniprotein, a Sso7d derived protein, an affibody, an affimer, an anticalin, an affilin, an affitin, a fynomer, or an Fc fusion molecule.
36. The antigen binding protein of embodiment 35, wherein the antigen binding protein is an antibody or an antigen binding fragment thereof.
37. The antigen binding protein of embodiment 36, wherein the antibody or an antigen binding fragment thereof comprises an Fc region.
38. The antigen binding protein of embodiment 36 or embodiment 37, wherein the antibody or an antigen binding fragment thereof is of the IgG, IgA, IgD, IgE or IgM isotype.
39. The antigen binding protein of any one of embodiments 36 to 38, wherein the antibody or an antigen binding fragment thereof is of the IgG1, IgG2, IgG3, or IgG4 isotype.
40. The antigen binding protein of any one of embodiments 36 to 39, wherein the antibody or an antigen binding fragment thereof is of the IgG1 or IgG4 isotype.
41. The antigen binding protein of any one of embodiments 36 to 40, wherein the antibody or an antigen binding fragment thereof is of the IgG1 isotype.
42. The antigen binding protein of any one of embodiments 36 to 41, wherein the antibody or an antigen binding fragment thereof further comprises a L234A substitution, a L235A substitution and/or a K409R substitution in its Fc region.
43. The antigen binding protein of embodiment 36, wherein the antibody or an antigen binding fragment thereof lacks an Fc region.
44. The antigen binding protein of any one of embodiments 36 to 43, wherein the antibody or an antigen binding fragment thereof is human.
45. The antigen binding protein of any one of embodiments 36 to 43, wherein the antibody or an antigen binding fragment thereof is humanized.
46. A polynucleotide encoding the antigen binding protein of any one of embodiments 34 to 45.
47. A vector comprising the polynucleotide of embodiment 46.
48. A host cell for producing an antigen binding protein comprising the vector of embodiment 47.
49. The cell of embodiment 48 wherein the cell is a hybridoma.
50. The cell of embodiment 48 wherein the antigen binding protein is recombinantly produced.
51. A method of producing an antigen binding protein, comprising culturing the host cell of any one of embodiments 48 to 50 under conditions such that the antigen binding protein is produced.
52. The inhibitor of any one of embodiments 1 to 33, wherein the inhibitor is non-proteinaceous.
53. The inhibitor of any one of embodiments 1 to 33 or 52, wherein the inhibitor is an aptamer.
54. The inhibitor of embodiment 52 or embodiment 53, wherein the inhibitor is an oligonucleotide aptamer.
55. The inhibitor of embodiment 52 or embodiment 53, wherein the inhibitor is a SOMamer.
56. The inhibitor of embodiment 53, wherein the inhibitor is a peptide aptamer.
57. A method for inhibiting an activity and/or function of gasdermin D, comprising contacting the gasdermin D inhibitor or antigen binding protein of any one of embodiments 1, 2, 5 to 45 or 52 to 56, with gasdermin D.
58. A method for neutralizing an activity and/or function of gasdermin D, comprising contacting the gasdermin D inhibitor or antigen binding protein of any one of embodiments 3 to 45 or 52 to 56, with gasdermin D.
59. Use of the gasdermin D inhibitor or antigen binding protein of any one of embodiments 1, 2, 5 to 45 or 52 to 56 in inhibiting an activity and/or function of gasdermin D.
60. Use of the gasdermin D inhibitor or antigen binding protein of any one of embodiments 3 to 45 or 52 to 56 in neutralizing an activity and/or function of gasdermin D.
61. The inhibitor or antigen binding protein of any one of embodiments 1 to 45 or 52 to 56, for use in inhibiting gasdermin D, such as neutralizing gasdermin D.
62. The inhibitor or antigen binding protein of any one of embodiments 1 to 45 or 52 to 56, for use in neutralizing gasdermin D.
63. The inhibitor or antigen binding protein of embodiment 61, for use in inhibiting gasdermin D, wherein the inhibition of gasdermin D is the inhibition of:
   i) an activity of gasdermin D, such as wherein the inhibitor neutralizes an activity of gasdermin D; and/or
   ii) a function of gasdermin D, such as wherein the inhibitor neutralizes a function of gasdermin D.
64. The inhibitor or antigen binding protein of embodiment 62, for use in neutralizing gasdermin D, wherein the neutralization of gasdermin D is the neutralization of:
   i) an activity of gasdermin D; and/or
   ii) a function of gasdermin D.
65. The inhibitor or antigen binding protein of any one of embodiments 1 to 45 or 52 to 56, for use in therapy.
66. The inhibitor or antigen binding protein of any one of embodiments 1 to 45 or 52 to 56, for use in the treatment of an indication selected from: sepsis, septic shock, non-alcoholic steatohepatitis, lung cancer, Familial Mediterranean Fever, autoinflammatory diseases, Cryoprin associated periodic syndromes, non-alcoholic fatty liver disease, Alzheimer's disease, Parkinson's disease, age related macular degeneration, atherosclerosis, asthma and allergy airway inflammation, gout, Crohn's, ulcerative colitis, inflammatory bowel disease, hypertension, nephropathy, myocardial infarction, multiple sclerosis, experimental autoimmune encephalitis, hyperinflammation following influenza infection, graft versus host disease, stroke, silicosis, asbestosis, mesothelioma, type 1 diabetes, type 2 diabetes, obesity-induced inflammation, insulin resistance, rheumatoid arthritis, myelodysplastic syndrome, contact hypersensitivity, joint inflammation triggered by chikungunya virus and traumatic brain injury.
67. A gasdermin D inhibitor that binds to gasdermin D and inhibits gasdermin D, such as wherein the inhibitor binds to gasdermin D and neutralizes gasdermin D, wherein the inhibition and/or neutralization of gasdermin D is the inhibition and/or neutralization of:
   i) an activity of gasdermin D, such as wherein the inhibitor neutralizes an activity of gasdermin D; and/or
   ii) a function of gasdermin D, such as wherein the inhibitor neutralizes a function of gasdermin D;
   and wherein:
   a) the inhibitor is an extracellular inhibitor;
   b) the inhibitor binds to the N-terminal domain of gasdermin D;
   c) the inhibitor binds to an epitope comprising SEQ ID NO: 9; and
   d) the inhibitor binds to SEQ ID NO: 9.
68. A method for inhibiting and/or neutralizing an activity and/or function of gasdermin D, comprising contacting a gasdermin D inhibitor with gasdermin D; wherein the gasdermin D inhibitor binds to gasdermin D and inhibits gasdermin D, such as wherein the inhibitor binds to gasdermin D and neutralizes gasdermin D, wherein the inhibition and/or neutralization of gasdermin D is the inhibition and/or neutralization of:
   i) an activity of gasdermin D, such as wherein the inhibitor neutralizes an activity of gasdermin D; and/or
   ii) a function of gasdermin D, such as wherein the inhibitor neutralizes a function of gasdermin D;
   and wherein:
   a) the inhibitor is an extracellular inhibitor;
   b) the inhibitor binds to the N-terminal domain of gasdermin D;
   c) the inhibitor binds to an epitope comprising SEQ ID NO: 9; and
   d) the inhibitor binds to SEQ ID NO: 9
69. Use of a gasdermin D inhibitor in inhibiting and/or neutralizing an activity and/or function of gasdermin D; wherein the gasdermin D inhibitor binds to gasdermin D and inhibits gasdermin D, such as wherein the inhibitor binds to gasdermin D and neutralizes gasdermin D, wherein the inhibition and/or neutralization of gasdermin D is the inhibition and/or neutralization of:
   i) an activity of gasdermin D, such as wherein the inhibitor neutralizes an activity of gasdermin D; and/or
   ii) a function of gasdermin D, such as wherein the inhibitor neutralizes a function of gasdermin D;
   and wherein:
   a) the inhibitor is an extracellular inhibitor;
   b) the inhibitor binds to the N-terminal domain of gasdermin D;
   c) the inhibitor binds to an epitope comprising SEQ ID NO: 9; and
   d) the inhibitor binds to SEQ ID NO: 9.
70. A gasdermin D inhibitor that binds to gasdermin D and inhibits gasdermin D, such as wherein the inhibitor binds to gasdermin D and neutralizes gasdermin D, wherein the inhibition and/or neutralization of gasdermin D is the inhibition and/or neutralization of:
   i) an activity of gasdermin D, such as wherein the inhibitor neutralizes an activity of gasdermin D; and/or
   ii) a function of gasdermin D, such as wherein the inhibitor neutralizes a function of gasdermin D;
   and wherein:
   a) the inhibitor is an extracellular inhibitor;
   b) the inhibitor binds to the N-terminal domain of gasdermin D;
   c) the inhibitor binds to an epitope comprising SEQ ID NO: 9; and
   d) the inhibitor binds to SEQ ID NO: 9;
      for use in therapy.
71. A gasdermin D inhibitor that binds to gasdermin D and inhibits gasdermin D, such as wherein the inhibitor binds to gasdermin D and neutralizes gasdermin D, wherein the inhibition and/or neutralization of gasdermin D is the inhibition and/or neutralization of:
   i) an activity of gasdermin D, such as wherein the inhibitor neutralizes an activity of gasdermin D; and/or
   ii) a function of gasdermin D, such as wherein the inhibitor neutralizes a function of gasdermin D;
   and wherein:
   a) the inhibitor is an extracellular inhibitor;
   b) the inhibitor binds to the N-terminal domain of gasdermin D;
   c) the inhibitor binds to an epitope comprising SEQ ID NO: 9; and
   d) the inhibitor binds to SEQ ID NO: 9;
      for use in the treatment of of an indication selected from: sepsis, septic shock, non-alcoholic steatohepatitis, lung cancer, Familial Mediterranean Fever, autoinflammatory diseases, Cryoprin associated periodic syndromes, non-alcoholic fatty liver disease, Alzheimer's disease, Parkinson's disease, age related macular degeneration, atherosclerosis, asthma and allergy airway inflammation, gout, Crohn's, ulcerative colitis, inflammatory bowel disease, hypertension, nephropathy, myocardial infarction, multiple sclerosis, experimental autoimmune encephalitis, hyperinflammation following influenza infection, graft versus host disease, stroke, silicosis, asbestosis, mesothelioma, type 1 diabetes, type 2 diabetes, obesity-induced inflammation, insulin resistance, rheumatoid arthritis, myelodysplastic syndrome, contact hypersensitivity, joint inflammation triggered by chikungunya virus and traumatic brain injury.

### BRIEF DESCRIPTION OF THE FIGURES

FIG. 1A presents a timecourse for a cell death inhibition assay. THP-1 cells were treated with nigericin (Nig) in combination with the small molecules 20 μM disulfiram (Dis), 30 μM necrosulfonamide (NSA), or 2 μM MCC-950 (MCC) or were treated with only the small molecules 20 μM disulfiram (Dis), 30 μM necrosulfonamide (NSA), or 2 μM MCC-950 (MCC). Cell death was measured by the uptake of SYTOX™ green.

FIG. 1B presents a cell death inhibition assay at 1 hour. THP-1 cells were treated with nigericin (Nig) in combination with the small molecules: 20 μM disulfiram (Dis), 30 μM necrosulfonamide (NSA), or 2 μM MCC-950 (MCC) or were treated with only the small molecules 20 μM disulfiram (Dis), 30 μM necrosulfonamide (NSA), or 2 μM MCC-950 (MCC). Cell death was measured by the uptake of SYTOX™ green after 1 hour.

FIGS. 2A and 2B present a timecourse for a cell death inhibition assay in the presence of the antibody, Ab5. THP-1 cells were treated with nigericin, nigericin in combination with MCC-950, the antibody Ab5, or combinations of nigericin and different concentrations of Ab5 (0.15 μg/ml-20 μg/ml). Cell death was measured by the uptake of SYTOX™ green.

FIG. 3 presents a representative image of SYTOXT™ green uptake after 1 hour in cells treated with A. mock B. nigericin C. nigericin and Ab5 and D. nigericin and MCC-950. Images were taken using an IncuCyte Live Cell Analysis system which allows for live cell fluorescent imaging.

FIG. 4 presents a timecourse for a cell death inhibition assay in the presence of the antibody, Ab3. THP-1 cells were treated with nigericin, nigericin in combination with MCC-950, the antibody Ab3, or combinations of nigericin and different concentrations of Ab3 (0.15 μg/ml-20 μg/ml). Cell death was measured by the uptake of SYTOX™ green.

FIG. 5 presents a representative image of SYTOX™ green uptake after 1 hour in cells treated with A. mock B. nigericin C. nigericin and other gasdermin D binding antibodies, such as Ab3, and D. nigericin and MCC-950. Images were taken using an IncuCyte Live Cell Analysis system which allows for live cell fluorescent imaging.

FIG. 6 presents a timecourse for a cell death inhibition assay in the presence of the antibody, Ab5, where Ab5 was boiled prior to administration to the THP-1 cells. THP-1 cells were treated with nigericin, nigericin in combination with MCC-950, and nigericin in combination with boiled Ab5. Cell death was measured by the uptake of SYTOX™ green.

FIG. 7A presents the protein stain on a blot of recombinant GSDMD before and after treatment with caspase-1. FIG. 7B presents a western blot of gasdermin D after treatment with caspase 1. Ab5 was used as the probe antibody.

FIG. 8 presents a timecourse for a cell death inhibition assay in the presence of Ab5 and a blocking peptide. THP-1 cells were treated with nigericin, nigericin in combination with MCC-950, nigericin in combination with Ab5, or nigericin in combination with Ab5 and a blocking peptide. Cell death was measured by the uptake of SYTOXM green.

FIG. 9 presents a representative image of SYTOX™ green uptake after 2 hours in cells treated with A. mock B. nigericin C. nigericin and Ab5 and D. nigericin and Ab5 and blocking peptide. Images were taken using an IncuCyte Live Cell Analysis system which allows for live cell fluorescent imaging.

FIG. 10A presents a timecourse for a cell death inhibition assay in the presence of Ab5 or a blocking peptide. THP-1 cells were treated with nigericin, nigericin in combination with Ab5, or nigericin in combination with a blocking peptide. Cell death was measured by the uptake of SYTOX™ green. FIGS. 10B and 1 OC present the quantification of IL-1β and IL-18 release in the supernatant 80 minutes after stimulation with nigericin.

FIG. 11 presents the quantification of IL-1β release from primary human monocytes in the presence of Ab5 or Ab5 and a blocking peptide. Primary human monocytes were primed for 3 hours with lipopolysaccharide (LPS) and then additionally treated with nigericin (LN), nigericin after the cells had been pre-treated with Ab5 (LN Ab ‘x’, where x is the concentration of antibody in μg/ml), or nigericin after the cells had been pretreated with Ab5 and 40 μg/ml blocking peptide (LNAb ‘x’+pep). IL-1β release into the supernatant was quantified 1 hour after stimulation with nigericin.

FIG. 12 presents a timecourse of a cell death inhibition assay in murine cells: Bone Marrow Derived Macrophages (BMDMs). Murine cells were primed with LPS and treated with nigericin or nigericin in combination with Ab5. Cell death was measured by the uptake of SYTOX™ green.

FIG. 13 presents a timecourse of a cell death inhibition assay and quantification of IL-1β release from murine bone marrow derived macrophages (BMDMs) after nigericin stimulation, in the presence of Ab5 or Ab5 and a blocking peptide. BMDMs were primed for 3 hours with lipopolysaccharide (LPS) and then additionally treated with nigericin, after the cells had been pre-treated with phosphate buffered saline (LN), or with Ab5 (LN Ab ‘x’, where x is the concentration of antibody in μg/ml), or with Ab5 and 40 μg/ml blocking peptide (LNAb ‘x’+pep). IL-1β release into the supernatant was quantified 1.5 hours after stimulation with nigericin (FIG. 13A) and the kinetics of cell death was measured by the uptake of SYTOX™ green (FIG. 13B).

FIG. 14 presents a timecourse of a cell death inhibition assay and quantification of IL-1β release from murine bone marrow derived macrophages (BMDMs) after anthrax toxin (LeTx) stimulation, in the presence of Ab5, or Ab5 and a blocking peptide. BMDMs were primed for 3 hours with lipopolysaccharide (LPS) and then additionally treated with LeTx, after the cells had been pre-treated with phosphate buffered saline (LLeTx), or with Ab5 (LLeTx Ab ‘x’, where x is the concentration of antibody in μg/ml), or with Ab5 and 40 μg/ml blocking peptide (LLeTxAb ‘x’+pep). IL-1β release into the supernatant was quantified 1.5 hours after stimulation with nigericin (FIG. 14A) and the kinetics of cell death was measured by the uptake of SYTOX™ green (FIG. 14B).

FIG. 15 presents a liposomal leakage assay. Recombinant gasdermin D was cleaved by caspase-1 and allowed to generate pores in calcein filled liposomes. The kinetics of calcein leakage out of the liposomes was recorded. FIG. 15 presents the kinetics of calcein leakage after the recombinant gasdermin D is pre-treated with varying concentrations of disulfiram (FIG. 15A), the gasdermin D specific antibody Ab5 (FIG. 15B) and other gasdermin D targeted antibodies that do not inhibit cell death or IL-1β release (FIG. 15C).

### EXAMPLES

### Example 1: Antibody Based Inhibition of Cell Death Induction

Anti-gasdermin D antibodies were tested to assess their ability to inhibit cell death induction upon NLRP3 inflammasome stimulation in a human monocyte cell line.

Human THP-1 cells are an immortalized monocyte-like cell line that are derived from the peripheral blood of acute monocytic leukaemia (M5 subtype). The THP-1 cells were cultured in RPMI medium supplemented with 10% FCS, sodium pyruvate and the antibiotics penicillin and streptomycin. Under maintenance/expansion culturing conditions these cells grow in suspension, however after overnight differentiation using phorbol 12-myristate 13-acetate these cells matured and became adherent. After the differentiation process it was possible to activate the NLRP3 inflammasome by stimulation with nigericin. This led to fast cell death induction (typically observed within 1-2 hours). This fast cell death induction (pyroptosis) is driven by GSDMD pore formation. Pyroptosis is a lytic form of cell death and can be monitored by measuring the increase in fluorescent signal of DNA dyes that cannot cross an intact cell membrane of live cells. In these examples SYTOX™ green is the dye that is used for detection, but the working principle is identical to the use of compounds like Propidium Iodide in FACS.

Human THP-1 cells, were cultured in the RPMI medium supplemented with 10% FCS, sodium pyruvate and the antibiotics penicillin and streptomycin. The cells were collected by centrifugation, resuspended, counted and finally plated in a 96 well plate, at a cell density of 50,000 cells per well in a total volume of 100 μl. The cells were incubated in RPMI medium supplemented with 10% FCS, sodium pyruvate and the antibiotics penicillin and streptomycin, with 100 ng/ml phorbol 12-myristate 13-acetate (PMA) for 16 hours overnight.

After overnight differentiation, the cells were adherent to the 96 well plate, and the medium was replaced with the RPMI medium supplemented with 10% FCS, sodium pyruvate and the antibiotics penicillin and streptomycin, without PMA, but including the fluorescent dye SYTOX™ green (1 μM final concentration). The cells were incubated for 2 hours prior to proceeding with the experiment.

The cells were pre-treated for 30 mins with 0.15-20 μg/ml of the gasdermin D targeted antibody or 20 μM of the small molecule disulfiram or 30 μM of the small molecule necrosulfonamide, prior to stimulation of the NLRP3 inflammasome by nigericin. After 30 minutes the activation of the NLRP3 inflammasome was stimulated by the addition of nigericin, at a concentration of 20 μM, to the cells.

A mock control was not induced with nigericin. In addition, a second negative control using 2 μM MCC-950 was also used. MCC-950 is a potent inhibitor of the NLRP3 inflammasome, and therefore human THP-1 cell lines that have been pre-treated for 30 minutes with MCC-950, prior to stimulation with nigericin, should not undergo NLRP3 inflammasome induced cell death. This control ensured that the observed cell death was indeed induced by the NLRP3 inflammasome and GSDMD pore formation. The small molecule inhibitors necrosulfonamide and disulfiram are known inhibitors of GSDMD pore formation, and were used as positive controls in this study.

The experiment described above was repeated, but the antibodies tested were denatured by boiling prior to administration to the Human THP-1 cell line. Boiling the antibodies will lead to denaturation and loss of binding to its target.

Induction of cell death was monitored by the uptake of SYTOX™ green, using an IncuCyte Live Cell Analysis system which allows for live cell fluorescent imaging. Cell death induction was monitored every 10 minutes, for 4 hours, and the imaged cells were analysed using the IncuCyte software to assess the percentage of cells that had taken up SYTOX™ green, and had therefore undergone cell death.

The 100% cell death fluorescent signal is measured as the maximum signal obtained from a Triton X-100 treated well, 0.1% final concentration of Triton X-100 will lyse all cells and induces staining of all cells. All fluorescence measurements were normalized to this value.

This experimental protocol can be used to perform a single readout of cell death at a particular timepoint, a preferred timepoint is after 1 hour, or can be used to follow the kinetics of cell death. All experiments used duplicates/triplicate measurements per condition.

Results:

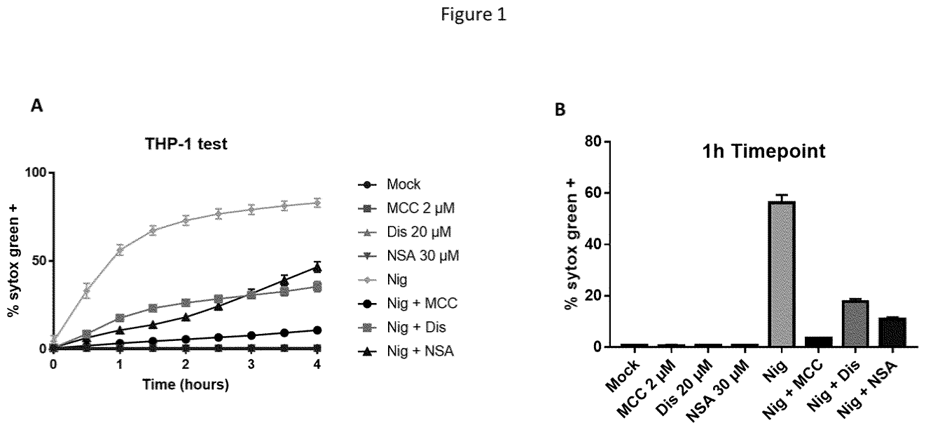

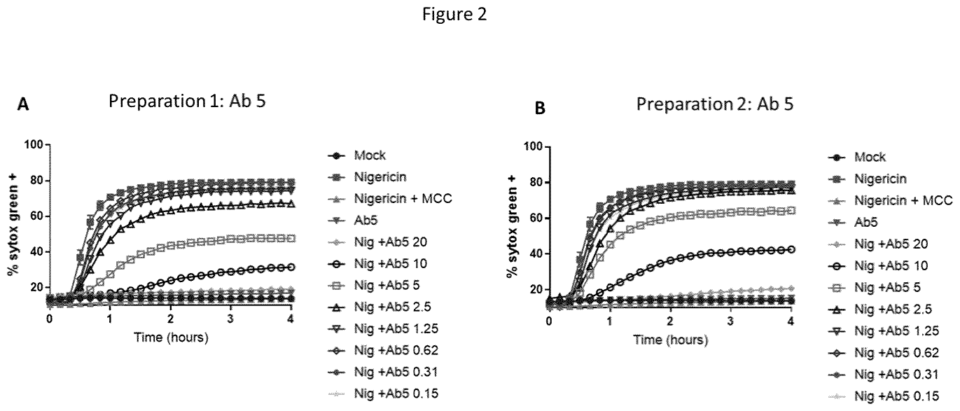

The incubation of 20 μM nigericin with human THP-1 resulted in the induction of cell death through the activation of the NLRP3 inflammasome (FIG. 1A, FIG. 1B, FIG. 2A, and FIG. 2B). Human THP-1 cells that had not been treated with nigericin (mock) or had been treated with a potent NLRP3 inhibitor (MCC-950), did not undergo cell death (FIG. 1A, FIG. 1B, FIG. 2A, and FIG. 2B).

The administration of the small molecule inhibitors, necrosulfonamide (30 μM) and disulfiram (20 μM), resulted in the inhibition and delay of cell death induction (FIGS. 1A and 1B), indicating that this experimental protocol could be used to measure inhibition of gasdermin D.

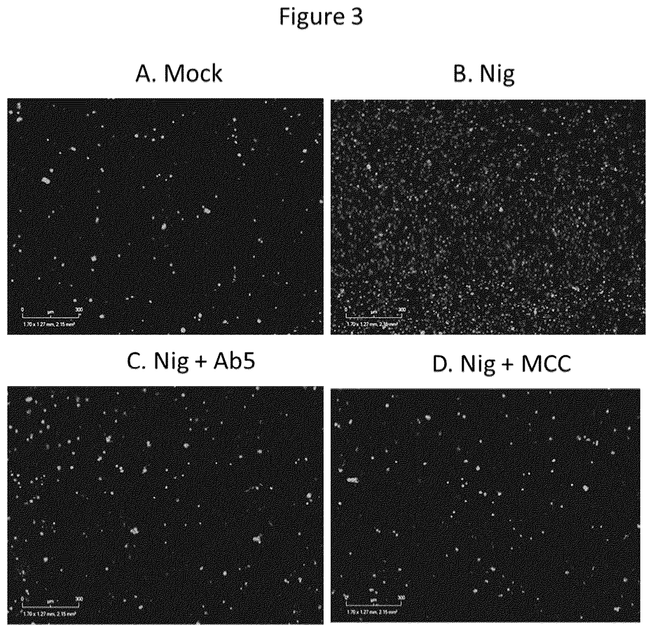

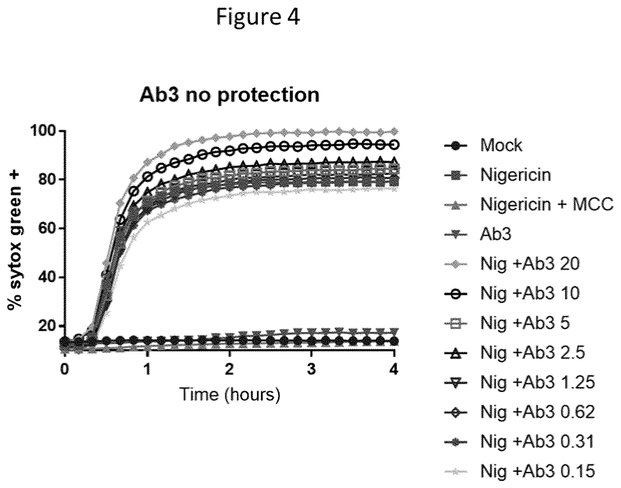

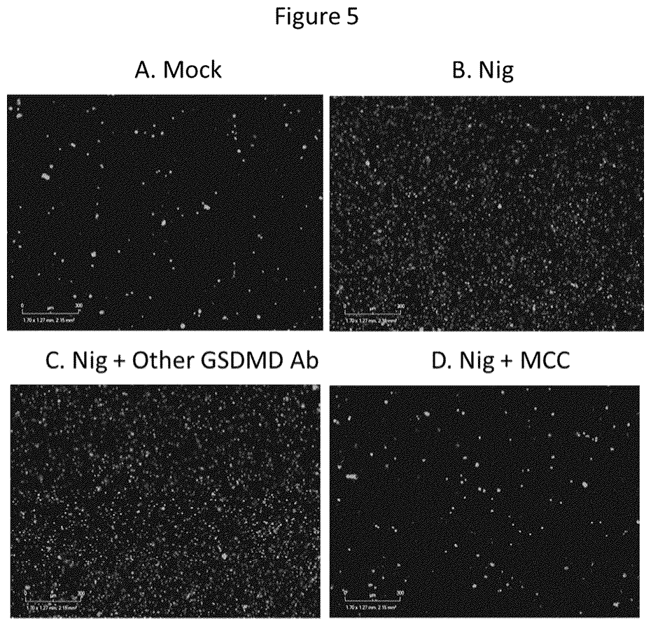

The administration of Ab5, which specifically targets gasdermin D, resulted in the inhibition and delay of cell death induction (FIG. 2A, FIG. 2B, and FIG. 3A-D). This activity is specific to Ab5, and the incubation of nigericin and other GSDMD antibodies, such as Ab3, with the Human THP-1 cell line, does not lead to an inhibition or delay in cell death (FIG. 4, and FIG. 5A-D).

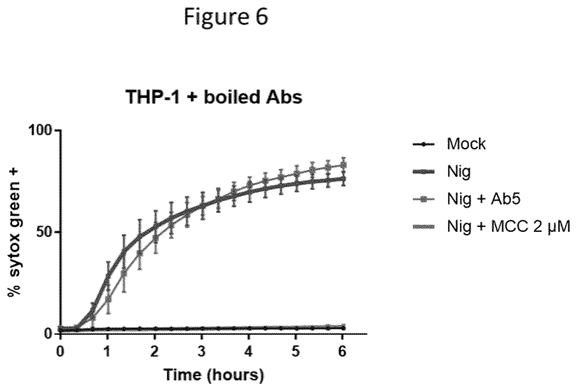

As shown in FIG. 6 boiling of the antibody prevents the inhibition of cell death induction, demonstrating that Ab5 must be in its native conformational state, where it is able to bind its target, in order to inhibit cell death induction.

### Example 2: Western Blot Analysis of Antibody Binding

Recombinant His-SUMO tagged gasdermin D (rGSDMD) was produced in E. coli. The material was purified by Ni/NTA affinity purification. This material was cleaved with Ulp-1 to remove the tag and size exclusion chromatography (SEC) was performed to improve purity.

The recombinant gasdermin D was incubated with recombinant caspase-1 for 1 hour at 37° C. The reaction mixture was run by SDS-PAGE prior to western blot. The western blot was incubated with Ab5, and a secondary antibody conjugated to horseradish peroxidase was used for detection.

Results:

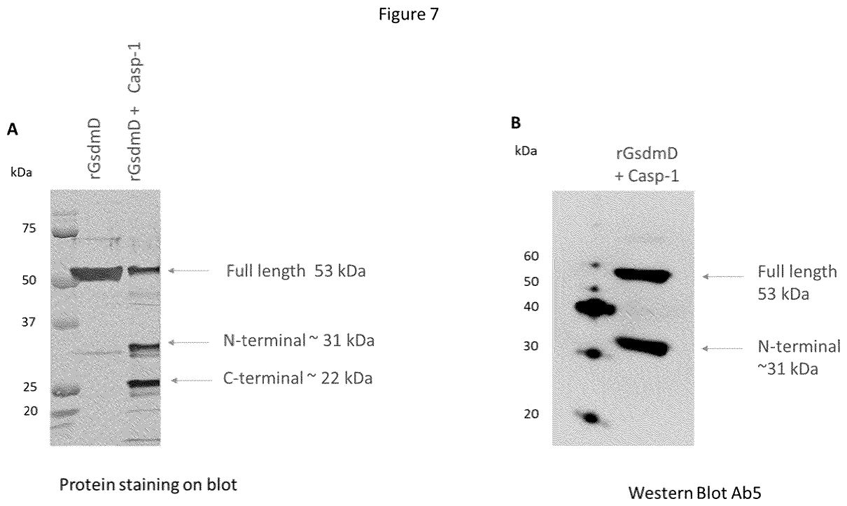

Cleavage of gasdermin D with caspase-1 resulted in the formation of a ˜31 kDa N-terminal domain and ˜22 kDa C-terminal domain (FIG. 7A). Western blot analysis of the cleavage reaction of recombinant gasdermin D, using Ab5 as a probe, indicated that Ab5 binds to the N-terminal domain of gasdermin D. As shown in FIG. 7B, the Ab5 is able to bind to either the full-length gasdermin D and the N-terminal domain of gasdermin D, but is unable to bind to the C-terminal domain.

### Example 3: Blocking Peptide Neutralizes Antibody Function

The experimental protocol described in Example 1 was used to determine the binding epitope of Ab5. Prior to the administration of the antibody to the THP-1 cells, the antibody (at a concentration of 20 μg/ml) was incubated with blocking peptides (at a concentration of 40 μg/ml) for 15 minutes. As a negative control, the THP-1 cells were also incubated with the blocking peptide (at a concentration of 40 μg/ml) in the absence of antibody.

The THP-1 cells were subsequently pre-treated for 30 mins with the antibody/peptide mixture prior to stimulation of the NLRP3 inflammasome by nigericin. After 30 minutes the activation of the NLRP3 inflammasome was stimulated by the addition of nigericin, at a concentration of 20 μM, to the cells. The extent of cell death was measured as described in Example 1. The release of IL-1β and IL-18 from the THP-1 cells into the supernatant was quantified 80 minutes after nigericin stimulation using the Luminex assay system according to the manufacturer's instructions.

Results:

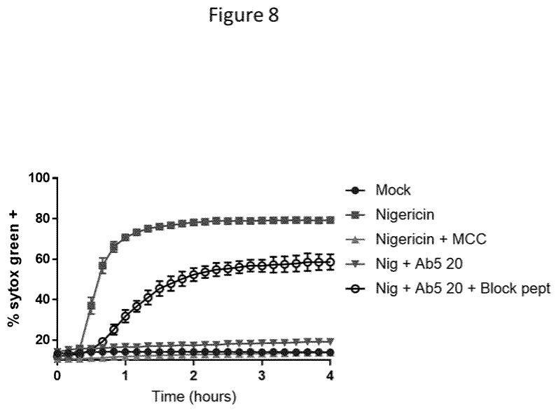

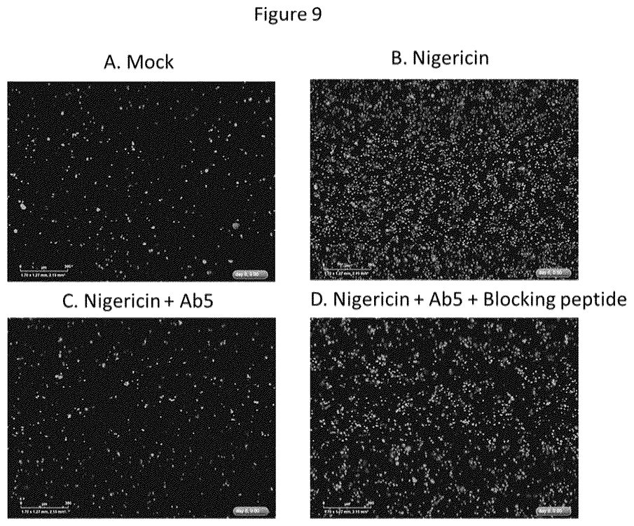

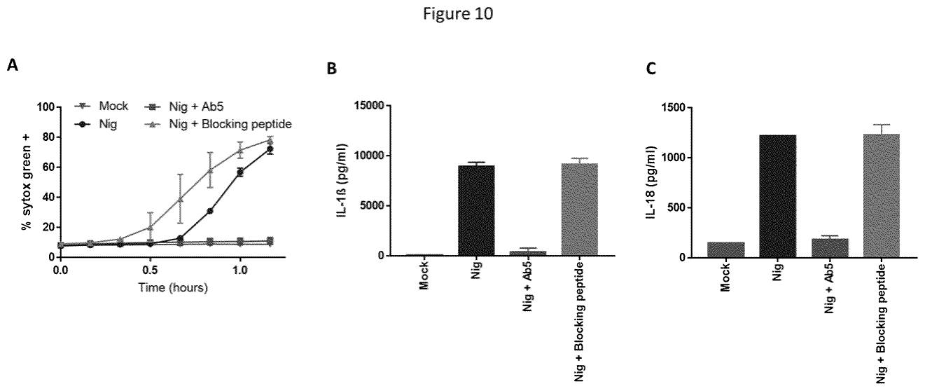

Incubation of the Ab5 with a peptide of sequence: KREGSGRFSLPGATC (SEQ ID NO: 9) prevented antibody dependent inhibition of cell death (FIG. 8 and FIG. 9A-D). The activity of this blocking peptide therefore indicates that Ab5 specifically binds to the sequence KREGSGRFSLPGATC (SEQ ID NO: 9) to inhibit an activity and/or function of gasdermin D. Incubation of the cells with blocking peptide alone does not prevent nigericin-induced cell death (FIG. 10A). In addition to cell death inhibition, Ab5 blocks cytokine (IL-1β and IL-18) release, while the incubation of Ab5 in the presence of blocking peptide has no effect on cytokine levels (FIGS. 10B and 10C).

### Example 4: Inhibition of IL-1p Release in Primary Human Monocytes

Anti-gasdermin D antibodies were tested to assess their ability to inhibit IL-1β release upon NLRP3 inflammasome stimulation in a primary human monocyte cell line.

Primary human monocytes (CD14+) were isolated from fresh human blood PBMCs using CD14 microbeads from Myltenyi Biotech, in accordance with the manufacturer's instructions. The primary human monocytes were kept frozen until used.

The cells were thawed, washed twice with fresh medium (RPMI supplemented with 10% FBS) and were primed for 3 hours with 100 ng/ml LPS. Prior to the stimulation with nigericin (15 μM) for an additional 1 hour to induce NLRP3 inflammasome activation, the cells were pre-treated for 15 minutes with a gasdermin D specific antibody (Ab5) at a concentration of between 0.03-0.5 μg/ml, or were pre-treated with gasdermin D specific antibody (Ab5) that had been incubated with an excess (40 μg/ml) of the peptide KREGSGRFSLPGATC. As a positive control, the cells were incubated with 100 ng/ml LPS and were pre-treated with phosphate buffered saline (PBS), as opposed to antibody, 15 minutes prior to stimulation with nigericin. As a negative control, the cells were incubated with LPS, but not incubated with nigericin.

The cell supernatant was collected 1 hour after treatment with nigericin and the release of IL-1β from the primary human monocyte cells into the supernatant was quantified using the Meso Scale Discovery (MSD) assay system according to the manufacturer's instructions.

Results:

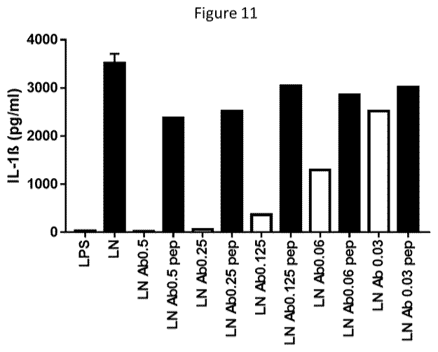

Primary human monocyte cells treated with LPS and nigericin (LN) released IL-1β into the supernatant (FIG. 11) suggesting that the gasdermin D pore is formed upon activation of the NLRP3 inflammasome. The mock control (LPS), where cells were treated with LPS only, did not result in the release of IL-1β (FIG. 11).

Pre-treatment of the primary human monocytes with antibody Ab5 (0.03-0.5 μg/ml), prior to stimulation with nigericin, lead to the inhibition of the release of IL-10 from these cells, in a dose dependent manner (white bars). In comparison, cells that had been pre-treated with antibody Ab5 that had been incubated with an excess of the blocking peptide (KREGSGRFSLPGATC), released IL-1β into the supernatant. This data is consistent with the data from THP-1 cell lines, and is indicative that the gasdermin D specific antibody is able to inhibit gasdermin D specific release of IL-1β from primary human monocytes.

### Example 5: Inhibition of Cell Death Induced by Pyroptosis in Murine Cells

The experimental protocol of Example 1 was repeated using murine cells (primary murine Bone Marrow Derived Macrophages (BMDMs) from Balb/C mice) to assess whether Ab5 is able to inhibit cell death induced by pyroptosis in murine cells.

Results:

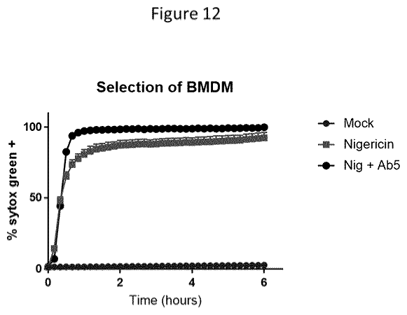

Incubation of the Ab5, at a concentration of 20 μg/ml, in the presence of nigericin in murine cells is unable to inhibit cell death by pyroptosis (FIG. 12). The high concentration of antibody (20 μg/ml) is toxic to primary murine BMDMs. As demonstrated in Example 6, lower concentration of the antibody (0.03-0.5 μg/ml) is tolerated well by the murine cells.

### Example 6: Inhibition of Cell Death and IL-1p Release Induced by Pyroptosis in Murine Cells

Anti-gasdermin D antibodies were tested to assess their ability to cross react with murine gasdermin D and inhibit cell death and IL-1β release upon NLRP3 or NLRP1B inflammasome stimulation in murine cells.

Balb/C mice express an anthrax lethal toxin (LeTx) sensitive NLRP1B allele. Incubation of the BMDM cells with LeTx therefore allows for specific activation of the NLRP1B inflammasome. This is analogous to the nigericin specific activation of NLRP3.

Bone marrow was isolated from the tibia and the femur of a Balb/C mouse. The cells were differentiated into bone marrow derived macrophages (BMDM) by culturing for 6 days in 10% FBS and penicillin/streptomycin supplemented IMDM medium mixed with L929 culture supernatant. After differentiation, cells were washed, collected and plated in fresh IMDM medium supplemented with 10% FBS and penicillin/streptomycin, at a density of 50,000 cells per well, in a 96 well plate and allowed to adhere overnight.

The cells were washed, and the medium was replaced and supplemented with 1 μM SYTOX™ green (ThermoFisher). This allows for the kinetic measurement of cell death induction using an Incucyte device (Essenbio) as described in Example 1. The cells were primed for 3 hours with 100 ng/ml LPS. Prior to stimulation of the NLRP3 or NLRP1B inflammasome, the cells were pre-treated for 15 minutes with gasdermin D specific antibody (Ab5) at a concentration of between 0.125-0.5 μg/ml, or were pre-treated with gasdermin D specific antibody (Ab5) that had been incubated with an excess (40 μg/ml) of the blocking peptide KREGSGRFSLPGATC. As a positive control, the cells were pre-treated for 15 minutes with PBS, as opposed to antibody, prior to inflammasome stimulation.

The cells were then stimulated with either the NLRP3 activator, nigericin (15 μM) or the NLRP1B activator, anthrax lethal toxin (LeTx)(1 μg/ml protective antigen (PA)+0.5 μg/ml lethal factor (LF)) for an additional 1.5 hours. As a negative control, the cells were incubated with LPS, but not incubated with nigericin or LeTx.

Induction of cell death was monitored by the uptake of SYTOX™ green, using an IncuCyte Live Cell Analysis system described in Example 1. Cell death induction was monitored every 10 minutes, for 1.5 hours, and the imaged cells were analysed using the IncuCyte software to assess the percentage of cells that had taken up SYTOX™ green, and had therefore undergone cell death. After 1.5 hours, the cell supernatant was collected and the release of IL-1β from the murine cells into the supernatant was quantified using the Luminex assay system according to the manufacturer's instructions.

Results:

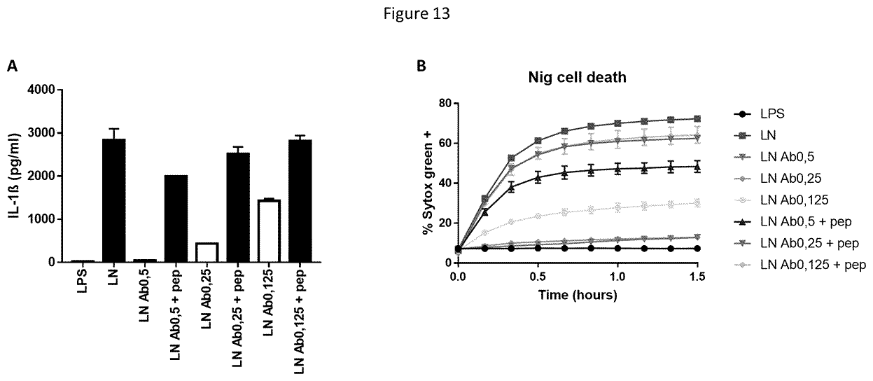

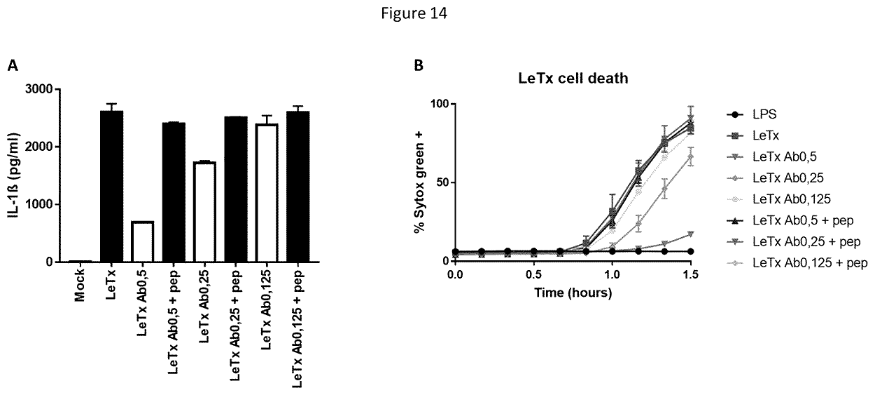

Murine cells treated with LPS and nigericin (LN), or LPS and LeTx (LLeTx), released IL-1β into the supernatant (FIG. 13A and FIG. 14A) and underwent cell death (FIG. 13B and FIG. 14B) upon activation of the NLRP3 and NLRP1B inflammasome. The mock control, where cells were treated with LPS only, did not result in the release of IL-1β or cell death (FIG. 13A, FIG. 13B, FIG. 14A, FIG. 14B).

Pre-treatment of the murine cells with antibody Ab5 (0.125-0.5 μg/ml), prior to stimulation with nigericin or LeTx, inhibited the release of IL-1β from these cells in a dose dependent manner and also inhibited cell death (FIG. 13A, FIG. 13B, FIG. 14A, FIG. 14B). This demonstrates that the anti-gasdermin D antibody Ab5, is able to cross-react with murine gasdermin D and inhibit gasdermin D dependent IL-1β release and cell death in murine cells.

The data presented in FIG. 14A and FIG. 14B also demonstrate that the murine gasdermin D is activated by the NLRP1B inflammasome, and that cell death and IL-1β release that is induced upon activation of the NLRP1B inflammasome can be inhibited by antibodies that target gasdermin D.

The murine cells that had been incubated with antibody Ab5, together with an excess of the blocking peptide (KREGSGRFSLPGATC), released IL-1β into the supernatant and underwent cell death upon NLRP3 or NLRP1B inflammasome activation (FIG. 13A, FIG. 13B, FIG. 14A and FIG. 14B).

### Example 7: Liposomal Leakage Assay

Recombinant gasdermin D (50 μg/ml, produced in-house) was pre-treated for 15 minutes with disulfiram (Sigma), the gasdermin D specific antibody Ab5, or a gasdermin D antibody rabbit polyclonal preparation that does not inhibit pyroptosis, in a HEPES buffer (20 mM HEPES, 150 mM NaCl). The mixture was added to a 384 well plate containing a mixture of calcein filled liposomes (POPE:POPS:POPC lipid composition, calcein encapsulated liposomes, Encapsula) and recombinant caspase-1 (10 μg/ml, Enzo Life Sciences), in HEPES buffer (20 mM HEPES, 150 mM NaCl). The cleavage of recombinant gasdermin D by recombinant caspase 1 results in insertion of gasdermin D into the liposome, and release of calcein. In the liposome, the calcein is at a concentration that leads to self quenching and it is therefore only detected upon release from the liposome.

Leakage of calcein out of the liposomes was measured every 3 minutes, for 42 minutes, using a Spectramax (Molecular Devices) with excitation set at 495 nm and emission at 515 nm.

Results:

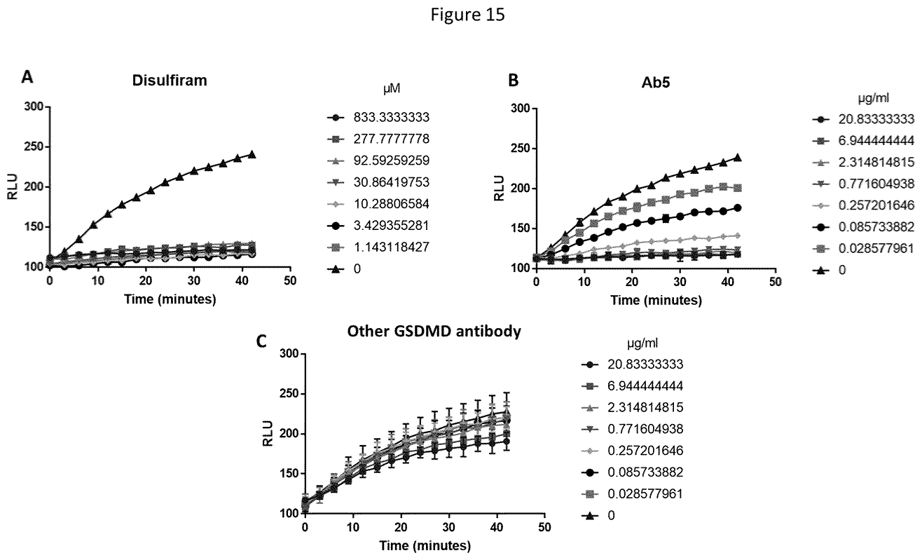

Pre-treatment of gasdermin D with disulfiram, a known gasdermin D pore blocking small molecule, potently blocked calcein leakage at all tested concentrations (FIG. 15A). Similarly, the pre-treatment of recombinant gasdermin D with Ab5, which is known to inhibit gasdermin D dependent cell death and IL-1β release (see Examples 1, 3, 4 and 6), also inhibited calcein leakage from the liposomes in a dose dependent manner (FIG. 15B). In comparison, pre-treatment of gasdermin D with a targeted antibody that is not able to inhibit pyroptosis, was also not able to block calcein release out of the liposomes (FIG. 15C). This suggests that in the presence of this non-inhibitory antibody, gasdermin D was efficiently cleaved by caspase-1 and was able to insert into the liposome and form a pore.

SEQUENCES
Gasdermin D full length
Sequence ID No: 1
MGSAFERVVRRVVQELDHGGEFIPVTSLQSSTGFQ
PYCLVVRKPSSSWFWKPRYKCVNLSIKDILEPDAA
EPDVQRGRSFHFYDAMDGQIQGSVELAAPGQAKIA
GGAAVSDSSSTSMNVYSLSVDPNTWQTLLHERHLR
QPEHKVLQQLRSRGDNVYVVTEVLQTQKEVEVTRT
HKREGSGRFSLPGATCLQGEGQGHLSQKKTVTIPS
GSTLAFRVAQLVIDSDLDVLLFPDKKQRTFQPPAT
GHKRSTSEGAWPQLPSGLSMMRCLHNFLTDGVPAE
GAFTEDFQGLRAEVETISKELELLDRELCQLLLEG
LEGVLRDQLALRALEEALEQGQSLGPVEPLDGPAG
AVLECLVLSSGMLVPELAIPVVYLLGALTMLSETQ
HKLLAEALESQTLLGPLELVGSLLEQSAPWQERST
MSLPPGLLGNSWGEGAPAWVLLDECGLELGEDTPH
VCWEPQAQGRMCALYASLALLSGLSQEPH
Gasdermin D N-terminal domain
Sequence ID No: 2
MGSAFERVVRRVVQELDHGGEFIPVTSLQSSTGFQ
PYCLVVRKPSSSWFWKPRYKCVNLSIKDILEPDAA
EPDVQRGRSFHFYDAMDGQIQGSVELAAPGQAKIA
GGAAVSDSSSTSMNVYSLSVDPNTWQTLLHERHLR
QPEHKVLQQLRSRGDNVYVVTEVLQTQKEVEVTRT
HKREGSGRFSLPGATCLQGEGQGHLSQKKTVTIPS
GSTLAFRVAQLVIDSDLDVLLFPDKKQRTFQPPAT
GHKRSTSEGAWPQLPSGLSMMRCLHNFLTD
Gasdermin D C-terminal domain
Sequence ID No: 3
GVPAEGAFTEDFQGLRAEVETISKELELLDRELCQ
LLLEGLEGVLRDQLALRALEEALEQGQSLGPVEPL
DGPAGAVLECLVLSSGMLVPELAIPVVYLLGALTM
LSETQHKLLAEALESQTLLGPLELVGSLLEQSAPW
QERSTMSLPPGLLGNSWGEGAPAWVLLDECGLELG
EDTPHVCWEPQAQGRMCALYASLALLSGLSQEPH
Fragment of gasdermin D N-terminal domain
Sequence ID No: 4
PNTWQTLLHERHLRQPEHKVLQQLRSRGDNVYVVT
EVLQTQKEVEVTRTHKREGSGRFSLPGATCLQGEG
QGHLSQKKTVTIPSGSTLAFRVAQLVIDSDLDVLL
FPDKKQRTFQ
Fragment of gasdermin D N-terminal domain
Sequence ID No: 5
RHLRQPEHKVLQQLRSRGDNVYVVTEVLQTQKEVE
VTRTHKREGSGRFSLPGATCLQGEGQGHLSQKKTV
TIPSGSTLAFRVAQLVIDSDLDVLL
Fragment of gasdermin D N-terminal domain
Sequence ID No: 6
LQQLRSRGDNVYVVTEVLQTQKEVEVTRTHKREGS
GRFSLPGATCLQGEGQGHLSQKKTVTIPSGSTLAF
RVAQL
Fragment of gasdermin D N-terminal domain
Sequence ID No: 7
VYVVTEVLQTQKEVEVTRTHKREGSGRFSLPGATC
LQGEGQGHLSQKKTVTIPSG
Fragment of gasdermin D N-terminal domain
Sequence ID No: 8
QKEVEVTRTHKREGSGRFSLPGATCLQGEGQGHLS
Fragment of gasdermin D N-terminal domain
Sequence ID No: 9
KREGSGRFSLPGATC
Fragment of gasdermin D N-terminal domain
Sequence ID No: 10
EHKVLQQLRSRGD
Fragment of gasdermin D N-terminal domain
Sequence ID No: 11
AFERVVRRVVQELD
Fragment of gasdermin D N-terminal domain
Sequence ID No: 12
YCLVVR
Fragment of gasdermin D N-terminal domain
Sequence ID No: 13
YKCVNLS
Fragment of gasdermin D N-terminal domain
Sequence ID No: 14
MDGQIQG
Fragment of gasdermin D N-terminal domain
Sequence ID No: 15
PNTWQTLLHE
Fragment of gasdermin D N-terminal domain
Sequence ID No: 16
DLDV
Murine gasdermin D fragment
Sequence ID No: 17
SQEGSGQFTLPGALC

1. A gasdermin D inhibitor that binds to gasdermin D and inhibits and/or neutralizes gasdermin D.

2. The gasdermin D inhibitor of claim 1, wherein the inhibitor neutralizes gasdermin D.

3. The gasdermin D inhibitor of claim 1, wherein the inhibition and/or neutralization of gasdermin D is the inhibition and/or neutralization of:
i) an activity of gasdermin D, such as wherein the inhibitor neutralizes an activity of gasdermin D; and/or
ii) a function of gasdermin D, such as wherein the inhibitor neutralizes a function of gasdermin D.

4. The inhibitor of claim 1, wherein the inhibitor:
i) is an extracellular inhibitor;
ii) binds to gasdermin D on the cell surface; and/or
iii) binds to gasdermin D and does not cross the cell membrane, unless it is bound to gasdermin D.

5. The inhibitor of claim 1, wherein the inhibitor is a large molecule, such as wherein the inhibitor has a molecular weight of >2 kDa, >3 kDa, >4 kDa, >5 kDa, >6 kDa, >7 kDa, >8 kDa, >9 kDa or >10 kDa.

6. The inhibitor of claim 1, wherein the inhibitor binds to the N-terminal domain of gasdermin D.

7. The inhibitor of claim 6, wherein the inhibitor binds to an epitope comprising SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the inhibitor binds to an epitope comprising SEQ ID NO: 9.

8. The inhibitor of claim 7, wherein the inhibitor binds to an epitope comprising SEQ ID NO: 9.

9. The inhibitor of claim 1, wherein the inhibitor binds to SEQ ID NO: 2, SEQ ID NO: 4, SEQ ID NO: 5, SEQ ID NO: 6, SEQ ID NO: 7, SEQ ID NO: 8, or SEQ ID NO: 9, such as wherein the inhibitor binds to SEQ ID NO: 9.

10. The inhibitor of claim 9, wherein the inhibitor binds to SEQ ID NO: 9.

11. The inhibitor of claim 1, wherein the inhibitor binds to gasdermin D and inhibits its association with lipids, such as wherein the inhibitor binds to gasdermin D and neutralizes the association of gasdermin D with lipids, optionally wherein the inhibitor binds to gasdermin D and inhibits its association with phosphatidylinositol 4-phosphate and/or phosphatidylinositol 4,5-bisphosphate, such as wherein the inhibitor binds to gasdermin D and neutralizes the association of gasdermin D with phosphatidylinositol 4-phosphate and/or phosphaidylinositol 4,5-bisphosphate.

12. The inhibitor of claim 1, wherein the inhibitor binds to gasdermin D and inhibits the oligomerisation of gasdermin D, such as wherein the inhibitor neutralizes oligomerisation of gasdermin D, optionally wherein the inhibitor inhibits protein-protein interactions between gasdermin D subunits, such as wherein the inhibitor neutralizes protein-protein interactions between gasdermin D subunits.

13. The inhibitor of claim 1, wherein the inhibitor binds to a gasdermin D multimeric pore, such as wherein:
i) the inhibitor blocks the pore; or
ii) the inhibitor disrupts protein-protein interactions between gasdermin D subunits of the pore.

14. The inhibitor of claim 13, wherein the inhibitor binds to a gasdermin D subunit of the multimeric pore, such as wherein:
i) the inhibitor blocks the pore; or
ii) the inhibitor disrupts protein-protein interactions between gasdermin D subunits of the pore.

15. The inhibitor of claim 1, wherein the inhibitor inhibits the release of IL-1β and/or IL-18.

16. The inhibitor of claim 1, wherein the inhibitor is proteinaceous, for example an antigen binding protein, such as wherein the antigen binding protein is an antibody or an antigen binding fragment thereof.

17. A method for inhibiting and/or neutralizing an activity and/or function of gasdermin D, comprising contacting the gasdermin D inhibitor of claim 1 with gasdermin D.

18-20. (canceled)

21. A method of treating an indication selected from sepsis, septic shock, non-alcoholic steatohepatitis, lung cancer, Familial Mediterranean Fever, autoinflammatory diseases, Cryoprin associated periodic syndromes, non-alcoholic fatty liver disease, Alzheimer's disease, Parkinson's disease, age related macular degeneration, atherosclerosis, asthma and allergy airway inflammation, gout, Crohn's, ulcerative colitis, inflammatory bowel disease, hypertension, nephropathy, myocardial infarction, multiple sclerosis, experimental autoimmune encephalitis, hyperinflammation following influenza infection, graft versus host disease, stroke, silicosis, asbestosis, mesothelioma, type 1 diabetes, type 2 diabetes, obesity-induced inflammation, insulin resistance, rheumatoid arthritis, myelodysplastic syndrome, contact hypersensitivity, joint inflammation triggered by chikungunya virus and traumatic brain injury, comprising administering the inhibitor of claim 1 to a subject having the indication.

22. A gasdermin D inhibitor that binds to gasdermin D and inhibits and/or neutralizes gasdermin D, wherein the inhibition and/or neutralization of gasdermin D is the inhibition and/or neutralization of:
i) an activity of gasdermin D, such as wherein the inhibitor neutralizes an activity of gasdermin D; and/or
ii) a function of gasdermin D, such as wherein the inhibitor neutralizes a function of gasdermin D;
and wherein:
a) the inhibitor is an extracellular inhibitor;
b) the inhibitor binds to the N-terminal domain of gasdermin D;
c) the inhibitor binds to an epitope comprising SEQ ID NO: 9; and
d) the inhibitor binds to SEQ ID NO: 9.

23. A method for inhibiting and/or neutralizing an activity and/or function of gasdermin D, comprising contacting a gasdermin D inhibitor with gasdermin D; wherein the gasdermin D inhibitor binds to gasdermin D and inhibits and/or neutralizes gasdermin D, wherein the inhibition and/or neutralization of gasdermin D is the inhibition and/or neutralization of:
i) an activity of gasdermin D, such as wherein the inhibitor neutralizes an activity of gasdermin D; and/or
ii) a function of gasdermin D, such as wherein the inhibitor neutralizes a function of gasdermin D;
and wherein:
a) the inhibitor is an extracellular inhibitor;
b) the inhibitor binds to the N-terminal domain of gasdermin D;
c) the inhibitor binds to an epitope comprising SEQ ID NO: 9; and
d) the inhibitor binds to SEQ ID NO: 9.

24-26. (canceled)
